SEQUENCE LISTING (1325 sequences)

SEQ ID NO: 1 - protein, 12 aa, Homo sapiens
DNGEKFESSGLR

SEQ ID NO: 2 - protein, 10 aa, Homo sapiens
TVQAVLTVPK

SEQ ID NO: 3 - protein, 20 aa, Homo sapiens
SDAEHCPELPPVDNSIFVAK

SEQ ID NO: 4 - protein, 12 aa, Homo sapiens
TSLEDFYLDEER

SEQ ID NO: 5 - protein, 8 aa, Homo sapiens
TEQAAVAR

SEQ ID NO: 6 - protein, 6 aa, Homo sapiens
KPRGQR

SEQ ID NO: 7 - protein, 9 aa, Homo sapiens
IDQTVEELR

SEQ ID NO: 8 - protein, 11 aa, Homo sapiens
SEDCFILDHGK

SEQ ID NO: 9 - protein, 7 aa, Homo sapiens
ILGPLSY

SEQ ID NO: 10 - protein, 7 aa, Homo sapiens
DMQFGNK

SEQ ID NO: 11 - protein, 6 aa, Homo sapiens
VIDAVR

SEQ ID NO: 12 - protein, 9 aa, Homo sapiens
VNSFFSTFK

SEQ ID NO: 13 - protein, 8 aa, Homo sapiens
TGAQELLR

SEQ ID NO: 14 - protein, 9 aa, Homo sapiens
IDQNVEELK

SEQ ID NO: 15 - protein, 15 aa, Homo sapiens
SEEPDPPPEPMSEER

SEQ ID NO: 16 - protein, 8 aa, Homo sapiens
FNALQYLR

SEQ ID NO: 17 - protein, 18 aa, Homo sapiens
EQVQSCGPPPELLNGNVK

SEQ ID NO: 18 - protein, 8 aa, Homo sapiens
LGAGSPMR

SEQ ID NO: 19 - protein, 6 aa, Homo sapiens
KSSVSK

SEQ ID NO: 20 - protein, 17 aa, Homo sapiens
ALYYDLISSPDIHGTYK

SEQ ID NO: 21 - protein, 12 aa, Homo sapiens
SLAPYAQDTQEK

SEQ ID NO: 22 - protein, 8 aa, Homo sapiens
PPQSPWDR

SEQ ID NO: 23 - protein, 13 aa, Homo sapiens
LDLQEINNWVQAQ

SEQ ID NO: 24 - protein, 6 aa, Homo sapiens
YSLELK

SEQ ID NO: 25 - protein, 27 aa, Homo sapiens
YTTFEYPNTISFSCNTGFYLNGADSAK

SEQ ID NO: 26 - protein, 15 aa, Homo sapiens
PPSVVVTSHQAPGEK

SEQ ID NO: 27 - protein, 11 aa, Homo sapiens
TQVNTQAEQLR

SEQ ID NO: 28 - protein, 8 aa, Homo sapiens
GEVQVSDK

SEQ ID NO: 29 - protein, 17 aa, Homo sapiens
QAAGLAFSDGDQWTLSR

SEQ ID NO: 30 - protein, 6 aa, Homo sapiens
ALIQPS

SEQ ID NO: 31 - protein, 6 aa, Homo sapiens
ALVQQM

SEQ ID NO: 32 - protein, 12 aa, Homo sapiens
VFAIPPSFASIF

SEQ ID NO: 33 - protein, 13 aa, Homo sapiens
AGALNSNDAFVLK

SEQ ID NO: 34 - protein, 9 aa, Homo sapiens
YSLNSGAVK

SEQ ID NO: 35 - protein, 13 aa, Homo sapiens
VHVSEEGTEPEAM

SEQ ID NO: 36 - protein, 8 aa, Homo sapiens
QAPGQNGT

SEQ ID NO: 37 - protein, 28 aa, Homo sapiens
SILTVSEEEWNTGETYTCVVAHEALPNR

SEQ ID NO: 38 - protein, 8 aa, Homo sapiens
QIQVSWLR

SEQ ID NO: 39 - protein, 28 aa, Homo sapiens
NGHSNQCQDGSGICVNCQHNTAGEHCER

SEQ ID NO: 40 - protein, 10 aa, Homo sapiens
ALVQQMEQLR

SEQ ID NO: 41 - protein, 9 aa, Homo sapiens
LAPLAEDVR

SEQ ID NO: 42 - protein, 11 aa, Homo sapiens
SLEDLQLTHNK

SEQ ID NO: 43 - protein, 18 aa, Homo sapiens
ALEYVCPSGFYPYPVQTR

SEQ ID NO: 44 - protein, 11 aa, Homo sapiens
KTKASPCDPTF

SEQ ID NO: 45 - protein, 13 aa, Homo sapiens
PNSMVVEHPEFLK

SEQ ID NO: 46 - protein, 9 aa, Homo sapiens
LTIGEGQQH

SEQ ID NO: 47 - protein, 13 aa, Homo sapiens
LGEVNTYAGDLQK

SEQ ID NO: 48 - protein, 11 aa, Homo sapiens
DPNGLPPEAQK

SEQ ID NO: 49 - protein, 11 aa, Homo sapiens
LIVHNGYCDGR

SEQ ID NO: 50 - protein, 7 aa, Homo sapiens
TPITVVK

SEQ ID NO: 51 - protein, 11 aa, Homo sapiens
TVAACNLPIVR

SEQ ID NO: 52 - protein, 6 aa, Homo sapiens
LDIIYK

SEQ ID NO: 53 - protein, 10 aa, Homo sapiens
YWGVASFLQK

SEQ ID NO: 54 - protein, 19 aa, Homo sapiens
DLSGMTWSHGLSVSKVLHK

SEQ ID NO: 55 - protein, 7 aa, Homo sapiens
ALDNLAR

SEQ ID NO: 56 - protein, 16 aa, Homo sapiens
DPDQTDGLGLSYLSSH

SEQ ID NO: 57 - protein, 10 aa, Homo sapiens
VEPYGENFNK

SEQ ID NO: 58 - protein, 26 aa, Homo sapiens
VKTYSCTNCQGNQPQTVGIENLGNLK

SEQ ID NO: 59 - protein, 17 aa, Homo sapiens
PDAELSASSVYNLLPEK

SEQ ID NO: 60 - protein, 14 aa, Homo sapiens
LAAAVSNFGYDLYR

SEQ ID NO: 61 - protein, 18 aa, Homo sapiens
RFNQPSDRHPEHYDTAIL

SEQ ID NO: 62 - protein, 9 aa, Homo sapiens
ISASAEELR

SEQ ID NO: 63 - protein, 10 aa, Homo sapiens
ICAMEGLPQK

SEQ ID NO: 64 - protein, 15 aa, Homo sapiens
INIMNLCEAGLLAPR

SEQ ID NO: 65 - protein, 14 aa, Homo sapiens
SLEYLDLSFNQIAR

SEQ ID NO: 66 - protein, 13 aa, Homo sapiens
CSTSSLLEACTFR

SEQ ID NO: 67 - protein, 13 aa, Homo sapiens
QPCQHGATCMPAG

SEQ ID NO: 68 - protein, 13 aa, Homo sapiens
TKSSMLPPKQALA

SEQ ID NO: 69 - protein, 8 aa, Homo sapiens
EPGLQIWR

SEQ ID NO: 70 - protein, 11 aa, Homo sapiens
GASQAGAPQGR

SEQ ID NO: 71 - protein, 12 aa, Homo sapiens
LQSLFDSPDFSK

SEQ ID NO: 72 - protein, 15 aa, Homo sapiens
ISETSLPPDMYECLR

SEQ ID NO: 73 - protein, 11 aa, Homo sapiens
HQGVMVGMGQK

SEQ ID NO: 74 - protein, 6 aa, Homo sapiens
VYIHPF

SEQ ID NO: 75 - protein, 6 aa, Homo sapiens
FIKAAI

SEQ ID NO: 76 - protein, 17 aa, Homo sapiens
SLAELGGHLDQQVEEFR

SEQ ID NO: 77 - protein, 11 aa, Homo sapiens
EIMENYNIALR

SEQ ID NO: 78 - protein, 6 aa, Homo sapiens
DLALPI

SEQ ID NO: 79 - protein, 11 aa, Homo sapiens
ETLFSVMPGLK

SEQ ID NO: 80 - protein, 6 aa, Homo sapiens
LMTPSK

SEQ ID NO: 81 - protein, 17 aa, Homo sapiens
QTQVSVLPEGGETPLFK

SEQ ID NO: 82 - protein, 10 aa, Homo sapiens
NNQIDHIDEK

SEQ ID NO: 83 - protein, 7 aa, Homo sapiens
AKLDVQF

SEQ ID NO: 84 - protein, 9 aa, Homo sapiens
LGVYELLLK

SEQ ID NO: 85 - protein, 18 aa, Homo sapiens
MADEAGSEADHEGTHSTK

SEQ ID NO: 86 - protein, 11 aa, Homo sapiens
RVEPYGENFNK

SEQ ID NO: 87 - protein, 8 aa, Homo sapiens
SELTQQLN

SEQ ID NO: 88 - protein, 14 aa, Homo sapiens
FDEFFSEGCAPGSK

SEQ ID NO: 89 - protein, 7 aa, Homo sapiens
MKTLLLF

SEQ ID NO: 90 - protein, 12 aa, Homo sapiens
GPDVLTATVSGK

SEQ ID NO: 91 - protein, 23 aa, Homo sapiens
EVSADQVATVMWDYFSQLSNNAK

SEQ ID NO: 92 - protein, 12 aa, Homo sapiens
AESERISLKLAN

SEQ ID NO: 93 - protein, 22 aa, Homo sapiens
AQPVQVAEGSEPDGFWEALGGK

SEQ ID NO: 94 - protein, 12 aa, Homo sapiens
IIDGMTYPGIIK

SEQ ID NO: 95 - protein, 13 aa, Homo sapiens
LNHQLEGLTFQMK

SEQ ID NO: 96 - protein, 8 aa, Homo sapiens
TASDFITK

SEQ ID NO: 97 - protein, 11 aa, Homo sapiens
EGGQTAPASTR

SEQ ID NO: 98 - protein, 11 aa, Homo sapiens
VLMSVLNDNSR

SEQ ID NO: 99 - protein, 11 aa, Homo sapiens
DYELLCLDGTR

SEQ ID NO: 100 - protein, 7 aa, Homo sapiens
GDVAFVK

SEQ ID NO: 101 - protein, 8 aa, Homo sapiens
IEISELNR

SEQ ID NO: 102 - protein, 10 aa, Homo sapiens
CVPDQDTAIR

SEQ ID NO: 103 - protein, 23 aa, Homo sapiens
VCTYFMPAAQLPELPDVELPTNK

SEQ ID NO: 104 - protein, 8 aa, Homo sapiens
DLDSQTMM

SEQ ID NO: 105 - protein, 7 aa, Homo sapiens
FSVVYAK

SEQ ID NO: 106 - protein, 8 aa, Homo sapiens
SGNTYLWR

SEQ ID NO: 107 - protein, 15 aa, Homo sapiens
NPDNDIRPWCFVLNR

SEQ ID NO: 108 - protein, 11 aa, Homo sapiens
DNDGWLTSDPR

SEQ ID NO: 109 - protein, 13 aa, Homo sapiens
FSSSSGYGGGSSR

SEQ ID NO: 110 - protein, 6 aa, Homo sapiens
SPSSPR

SEQ ID NO: 111 - protein, 15 aa, Homo sapiens
LPEIAIPEFIIPTLN

SEQ ID NO: 112 - protein, 9 aa, Homo sapiens
NGNLQYDLH

SEQ ID NO: 113 - protein, 8 aa, Homo sapiens
IVIEYVDR

SEQ ID NO: 114 - protein, 16 aa, Homo sapiens
EGTCPEAPTDECKPVK

SEQ ID NO: 115 - protein, 7 aa, Homo sapiens
AVQYSCR

SEQ ID NO: 116 - protein, 8 aa, Homo sapiens
VQVLLGAH

SEQ ID NO: 117 - protein, 8 aa, Homo sapiens
TYNVLDMK

SEQ ID NO: 118 - protein, 33 aa, Homo sapiens
NVQFNYPHTSVTDVTQNNFHNYFGGSEIVVAGK

SEQ ID NO: 119 - protein, 15 aa, Homo sapiens
CVLFPYGGCQGNGNK

SEQ ID NO: 120 - protein, 7 aa, Homo sapiens
VQVVAGK

SEQ ID NO: 121 - protein, 11 aa, Homo sapiens
SSGSLLNNAIK

SEQ ID NO: 122 - protein, 11 aa, Homo sapiens
LLPHANEVSQK

SEQ ID NO: 123 - protein, 15 aa, Homo sapiens
LGWDDDYWSVDPLDR

SEQ ID NO: 124 - protein, 19 aa, Homo sapiens
PYPNNFETTTVITVPTGYR

SEQ ID NO: 125 - protein, 7 aa, Homo sapiens
LLNIQTY

SEQ ID NO: 126 - protein, 15 aa, Homo sapiens
SLPVSDSVLSGFEQR

SEQ ID NO: 127 - protein, 11 aa, Homo sapiens
TQDPVPPGKPS

SEQ ID NO: 128 - protein, 16 aa, Homo sapiens
QVGSGVTTDQVQAEAK

SEQ ID NO: 129 - protein, 15 aa, Homo sapiens
EVQGFESATFLGYFK

SEQ ID NO: 130 - protein, 14 aa, Homo sapiens
YLSDHSFLVSQGDR

SEQ ID NO: 131 - protein, 14 aa, Homo sapiens
SSGLVSNAPGVQIR

SEQ ID NO: 132 - protein, 7 aa, Homo sapiens
MIGEVIR

SEQ ID NO: 133 - protein, 8 aa, Homo sapiens
NVVFVIDK

SEQ ID NO: 134 - protein, 7 aa, Homo sapiens
LGEVNTY

SEQ ID NO: 135 - protein, 10 aa, Homo sapiens
DGAGDVAFVK

SEQ ID NO: 136 - protein, 15 aa, Homo sapiens
IECVSAETTEDCIAK

SEQ ID NO: 137 - protein, 12 aa, Homo sapiens
GQGTLSVVTMYH

SEQ ID NO: 138 - protein, 10 aa, Homo sapiens
VIPACLPSPN

SEQ ID NO: 139 - protein, 11 aa, Homo sapiens
TVQLDDYLNGR

SEQ ID NO: 140 - protein, 8 aa, Homo sapiens
QEELCLAR

SEQ ID NO: 141 - protein, 14 aa, Homo sapiens
AESPEVCFNEESPK

SEQ ID NO: 142 - protein, 10 aa, Homo sapiens
YGLDSDLSCK

SEQ ID NO: 143 - protein, 16 aa, Homo sapiens
EIPAWVPFDPAAQITK

SEQ ID NO: 144 - protein, 16 aa, Homo sapiens
NGTQVNVPFITGLATK

SEQ ID NO: 145 - protein, 9 aa, Homo sapiens
WFYIASAFR

SEQ ID NO: 146 - protein, 9 aa, Homo sapiens
VLVNYIFFK

SEQ ID NO: 147 - protein, 6 aa, Homo sapiens
WVMVPM

SEQ ID NO: 148 - protein, 12 aa, Homo sapiens
QLNVIDNQRTLS

SEQ ID NO: 149 - protein, 11 aa, Homo sapiens
LNHQLEGLTFQ

SEQ ID NO: 150 - protein, 17 aa, Homo sapiens
SVSLVADENPFAQGALK

SEQ ID NO: 151 - protein, 10 aa, Homo sapiens
AGEVQEPELR

SEQ ID NO: 152 - protein, 20 aa, Homo sapiens
SLPAPWLSMAPVSWITPGLK

SEQ ID NO: 153 - protein, 9 aa, Homo sapiens
SSFVAPLEK

SEQ ID NO: 154 - protein, 6 aa, Homo sapiens
FKLLTL

SEQ ID NO: 155 - protein, 17 aa, Homo sapiens
QDPPSVVVTSHQAPGEK

SEQ ID NO: 156 - protein, 9 aa, Homo sapiens
QNVLGEFER

SEQ ID NO: 157 - protein, 11 aa, Homo sapiens
ENFLFLTPDCK

SEQ ID NO: 158 - protein, 19 aa, Homo sapiens
NLTILQEQSQIQRVILGAK

SEQ ID NO: 159 - protein, 12 aa, Homo sapiens
DTDTGALLFIGK

SEQ ID NO: 160 - protein, 9 aa, Homo sapiens
ELLETVVNR

SEQ ID NO: 161 - protein, 10 aa, Homo sapiens
VFAIPPSFAS

SEQ ID NO: 162 - protein, 10 aa, Homo sapiens
SVPMVPPGIK

SEQ ID NO: 163 - protein, 6 aa, Homo sapiens
EAMALA

SEQ ID NO: 164 - protein, 7 aa, Homo sapiens
ESLSRQR

SEQ ID NO: 165 - protein, 16 aa, Homo sapiens
SPEQQETVLDGNLIIR

SEQ ID NO: 166 - protein, 8 aa, Homo sapiens
ASYLDCIR

SEQ ID NO: 167 - protein, 10 aa, Homo sapiens
VGDTLNLNLR

SEQ ID NO: 168 - protein, 21 aa, Homo sapiens
LFHTNFYDTVGTIQLINDHVK

SEQ ID NO: 169 - protein, 7 aa, Homo sapiens
VSQEGLK

SEQ ID NO: 170 - protein, 17 aa, Homo sapiens
GECVPGEQEPEPILIPR

SEQ ID NO: 171 - protein, 12 aa, Homo sapiens
VGSALFLSHNLK

SEQ ID NO: 172 - protein, 9 aa, Homo sapiens
LSYEGEVTK

SEQ ID NO: 173 - protein, 13 aa, Homo sapiens
NVPLPVIAELPPK

SEQ ID NO: 174 - protein, 11 aa, Homo sapiens
YNALDLTNNGK

SEQ ID NO: 175 - protein, 6 aa, Homo sapiens
FIVDLT

SEQ ID NO: 176 - protein, 13 aa, Homo sapiens
MMTGFAPDTDDLK

SEQ ID NO: 177 - protein, 12 aa, Homo sapiens
PALPAGTEDTAK

SEQ ID NO: 178 - protein, 10 aa, Homo sapiens
WLQGSQELPR

SEQ ID NO: 179 - protein, 7 aa, Homo sapiens
MQQLEQM

SEQ ID NO: 180 - protein, 16 aa, Homo sapiens
TMEQFTIHLTVNPQSK

SEQ ID NO: 181 - protein, 18 aa, Homo sapiens
ENADSLQASLRPHADELK

SEQ ID NO: 182 - protein, 12 aa, Homo sapiens
GPEVQLVAHSPW

SEQ ID NO: 183 - protein, 14 aa, Homo sapiens
EDPQTFYYAVAVVK

SEQ ID NO: 184 - protein, 8 aa, Homo sapiens
VTSTLTIK

SEQ ID NO: 185 - protein, 12 aa, Homo sapiens
DQYELLCLDNTR

SEQ ID NO: 186 - protein, 8 aa, Homo sapiens
DDTVCLAK

SEQ ID NO: 187 - protein, 15 aa, Homo sapiens
EIPDEISILLLGVAH

SEQ ID NO: 188 - protein, 10 aa, Homo sapiens
GHFSISIPVK

SEQ ID NO: 189 - protein, 23 aa, Homo sapiens
SLGPNSCSANGPGLYLIHGPNLY

SEQ ID NO: 190 - protein, 17 aa, Homo sapiens
NPGCVVVDGMPPGVSFK

SEQ ID NO: 191 - protein, 10 aa, Homo sapiens
FVTWIEGMMR

SEQ ID NO: 192 - protein, 14 aa, Homo sapiens
TEVSSNHVLIYLDK

SEQ ID NO: 193 - protein, 14 aa, Homo sapiens
EVEGQILGTYVCIK

SEQ ID NO: 194 - protein, 25 aa, Homo sapiens
FGMHFCGGTLISPEWVLTAAHCLEK

SEQ ID NO: 195 - protein, 11 aa, Homo sapiens
ITLLSALVETR

SEQ ID NO: 196 - protein, 11 aa, Homo sapiens
CEGFVCAQTGR

SEQ ID NO: 197 - protein, 19 aa, Homo sapiens
SAGWNIPIGLLYCDLPEPR

SEQ ID NO: 198 - protein, 10 aa, Homo sapiens
PNMIDAATLK

SEQ ID NO: 199 - protein, 10 aa, Homo sapiens
NNLELSTPLK

SEQ ID NO: 200 - protein, 15 aa, Homo sapiens
HLDSVLQQLQTEVYR

SEQ ID NO: 201 - protein, 15 aa, Homo sapiens
GNDDHWIVDTDYDTY

SEQ ID NO: 202 - protein, 11 aa, Homo sapiens
SQNELQNQLDK

SEQ ID NO: 203 - protein, 18 aa, Homo sapiens
DVDECSLKPSICGTAVCK

SEQ ID NO: 204 - protein, 19 aa, Homo sapiens
AVLDVFEEGTEASAATAVK

SEQ ID NO: 205 - protein, 14 aa, Homo sapiens
NGMQFSTWDNDNDK

SEQ ID NO: 206 - protein, 11 aa, Homo sapiens
DKVNSFFSTFK

SEQ ID NO: 207 - protein, 17 aa, Homo sapiens
EDLIWELLNQAQEHFGK

SEQ ID NO: 208 - protein, 13 aa, Homo sapiens
SGNSVAALVFQKM

SEQ ID NO: 209 - protein, 13 aa, Homo sapiens
AGDVEGHLSFLEK

SEQ ID NO: 210 - protein, 24 aa, Homo sapiens
GLSDEAVTSLLPQLIEVSSPITLQ

SEQ ID NO: 211 - protein, 11 aa, Homo sapiens
AGHIAWTSSGK

SEQ ID NO: 212 - protein, 8 aa, Homo sapiens
LMCAESNR

SEQ ID NO: 213 - protein, 7 aa, Homo sapiens
QRDLIGK

SEQ ID NO: 214 - protein, 9 aa, Homo sapiens
AGGAPAAPQ

SEQ ID NO: 215 - protein, 6 aa, Homo sapiens
YQPKVK

SEQ ID NO: 216 - protein, 12 aa, Homo sapiens
ALGSLNDLQFFR

SEQ ID NO: 217 - protein, 7 aa, Homo sapiens
EGAASQK

SEQ ID NO: 218 - protein, 25 aa, Homo sapiens
VSNGAGTMSVSLVADENPFAQGALK

SEQ ID NO: 219 - protein, 13 aa, Homo sapiens
GLETSLAECTFTK

SEQ ID NO: 220 - protein, 11 aa, Homo sapiens
GLCVATPVQLR

SEQ ID NO: 221 - protein, 12 aa, Homo sapiens
TYMLAFDVNDEK

SEQ ID NO: 222 - protein, 11 aa, Homo sapiens
LLLENGAGINT

SEQ ID NO: 223 - protein, 8 aa, Homo sapiens
KTINDIFK

SEQ ID NO: 224 - protein, 12 aa, Homo sapiens
ELSGIPLNMAPK

SEQ ID NO: 225 - protein, 15 aa, Homo sapiens
LLSEPINIIDALEMR

SEQ ID NO: 226 - protein, 22 aa, Homo sapiens
DPDQTDGLGLSYLSSHIANVER

SEQ ID NO: 227 - protein, 22 aa, Homo sapiens
CSLLVLENELNAELGLSGASMK

SEQ ID NO: 228 - protein, 14 aa, Homo sapiens
EAEAFLEKYGYLNE

SEQ ID NO: 229 - protein, 7 aa, Homo sapiens
SVQEIQA

SEQ ID NO: 230 - protein, 12 aa, Homo sapiens
HYDGSYSTFGER

SEQ ID NO: 231 - protein, 7 aa, Homo sapiens
DLLYIGK

SEQ ID NO: 232 - protein, 7 aa, Homo sapiens
GLTAIPA

SEQ ID NO: 233 - protein, 16 aa, Homo sapiens
QQQPPRLPPEVEAGQR

SEQ ID NO: 234 - protein, 7 aa, Homo sapiens
NVIFSPL

SEQ ID NO: 235 - protein, 6 aa, Homo sapiens
YSNPDR

SEQ ID NO: 236 - protein, 12 aa, Homo sapiens
ASSIIDELFQDR

SEQ ID NO: 237 - protein, 12 aa, Homo sapiens
YQAVDLDECASR

SEQ ID NO: 238 - protein, 9 aa, Homo sapiens
SVNDLYIQK

SEQ ID NO: 239 - protein, 9 aa, Homo sapiens
IYLQNGPDR

SEQ ID NO: 240 - protein, 6 aa, Homo sapiens
LSMEIK

SEQ ID NO: 241 - protein, 29 aa, Homo sapiens
TAMDVFVCTYFMPAAQLPELPDVELPTNK

SEQ ID NO: 242 - protein, 12 aa, Homo sapiens
GVCEETSGAYEK

SEQ ID NO: 243 - protein, 11 aa, Homo sapiens
SASDLTWDNLK

SEQ ID NO: 244 - protein, 7 aa, Homo sapiens
LINDYVK

SEQ ID NO: 245 - protein, 18 aa, Homo sapiens
VLSIAQAHSPAFSCEQVR

SEQ ID NO: 246 - protein, 17 aa, Homo sapiens
EIPDEISILLLGVAHFK

SEQ ID NO: 247 - protein, 16 aa, Homo sapiens
SVVDENFSWYLEDNIK

SEQ ID NO: 248 - protein, 7 aa, Homo sapiens
DVVLFEK

SEQ ID NO: 249 - protein, 10 aa, Homo sapiens
FFTSHNGMQF

SEQ ID NO: 250 - protein, 6 aa, Homo sapiens
ITDLIK

SEQ ID NO: 251 - protein, 8 aa, Homo sapiens
MTDLTNNK

SEQ ID NO: 252 - protein, 8 aa, Homo sapiens
LCDNLSTK

SEQ ID NO: 253 - protein, 9 aa, Homo sapiens
AALAAFNAQ

SEQ ID NO: 254 - protein, 16 aa, Homo sapiens
CTTPPPSSGPTYQCLK

SEQ ID NO: 255 - protein, 14 aa, Homo sapiens
TENCAVLSGAANGK

SEQ ID NO: 256 - protein, 9 aa, Homo sapiens
SPELQAEAK

SEQ ID NO: 257 - protein, 22 aa, Homo sapiens
SFPEQPDRTDLVKELLFDAIGR

SEQ ID NO: 258 - protein, 18 aa, Homo sapiens
TQFNNNEYSQDLDAYNTK

SEQ ID NO: 259 - protein, 11 aa, Homo sapiens
TRLAEDPAKPR

SEQ ID NO: 260 - protein, 7 aa, Homo sapiens
QSWSVCK

SEQ ID NO: 261 - protein, 10 aa, Homo sapiens
ADLSGITGAR

SEQ ID NO: 262 - protein, 11 aa, Homo sapiens
EMNGEPLAAEK

SEQ ID NO: 263 - protein, 18 aa, Homo sapiens
VSVQLEASPAFLAVPVEK

SEQ ID NO: 264 - protein, 11 aa, Homo sapiens
MTVTDQVNCPK

SEQ ID NO: 265 - protein, 11 aa, Homo sapiens
DTVQIHDITGK

SEQ ID NO: 266 - protein, 22 aa, Homo sapiens
QGPVNLLSDPEQGVEVTGQYER

SEQ ID NO: 267 - protein, 12 aa, Homo sapiens
DLDSQTMMVLVN

SEQ ID NO: 268 - protein, 7 aa, Homo sapiens
INYTNEK

SEQ ID NO: 269 - protein, 14 aa, Homo sapiens
VCPFAGILENGAVR

SEQ ID NO: 270 - protein, 26 aa, Homo sapiens
QRPPDLDTSSNAVDLLFFTDESGDSR

SEQ ID NO: 271 - protein, 14 aa, Homo sapiens
SELTQQLNALFQDK

SEQ ID NO: 272 - protein, 7 aa, Homo sapiens
FGMNIGK

SEQ ID NO: 273 - protein, 7 aa, Homo sapiens
YWGVASF

SEQ ID NO: 274 - protein, 19 aa, Homo sapiens
SDNCEDTPEAGYFAVAVVK

SEQ ID NO: 275 - protein, 21 aa, Homo sapiens
DANEDSKMGSTVTAAVIGIIV

SEQ ID NO: 276 - protein, 15 aa, Homo sapiens
VFAIPPSFASIFLTK

SEQ ID NO: 277 - protein, 12 aa, Homo sapiens
TSADGQAGNEEM

SEQ ID NO: 278 - protein, 17 aa, Homo sapiens
NKPGVYTDVAYYLAWIR

SEQ ID NO: 279 - protein, 14 aa, Homo sapiens
EKQLQQELLLIQQQ

SEQ ID NO: 280 - protein, 21 aa, Homo sapiens
HVEDVPAFQALGSLNDLQFFR

SEQ ID NO: 281 - protein, 16 aa, Homo sapiens
ATFQTPDFIVPLTDLR

SEQ ID NO: 282 - protein, 15 aa, Homo sapiens
NSCPPTSELLGTSDR

SEQ ID NO: 283 - protein, 18 aa, Homo sapiens
TLDEFTIIQNLQPQYQFR

SEQ ID NO: 284 - protein, 12 aa, Homo sapiens
IHLISTQSAIPY

SEQ ID NO: 285 - protein, 14 aa, Homo sapiens
TIEGPLTSFGLSNK

SEQ ID NO: 286 - protein, 26 aa, Homo sapiens
LNDLNSVLVMPTFHVPFTDLQVPSCK

SEQ ID NO: 287 - protein, 7 aa, Homo sapiens
ECLQTCR

SEQ ID NO: 288 - protein, 11 aa, Homo sapiens
TGLLLLSDPDK

SEQ ID NO: 289 - protein, 31 aa, Homo sapiens
GNPEQTPVLKPEEEAPAPEVGASKPEGIDSR

SEQ ID NO: 290 - protein, 12 aa, Homo sapiens
NEDSLVFVQTDK

SEQ ID NO: 291 - protein, 10 aa, Homo sapiens
CLAYDFYPGK

SEQ ID NO: 292 - protein, 8 aa, Homo sapiens
PVNPVEQR

SEQ ID NO: 293 - protein, 14 aa, Homo sapiens
VKEHQESMDMNNPQ

SEQ ID NO: 294 - protein, 14 aa, Homo sapiens
QTMSLPEKSQSQIK

SEQ ID NO: 295 - protein, 6 aa, Homo sapiens
MSCAGR

SEQ ID NO: 296 - protein, 9 aa, Homo sapiens
TFPGFFSPM

SEQ ID NO: 297 - protein, 7 aa, Homo sapiens
QLGLQAK

SEQ ID NO: 298 - protein, 12 aa, Homo sapiens
YLQEIYNSNNQK

SEQ ID NO: 299 - protein, 9 aa, Homo sapiens
YIETDPANR

SEQ ID NO: 300 - protein, 11 aa, Homo sapiens
INHGILYDEEK

SEQ ID NO: 301 - protein, 8 aa, Homo sapiens
LEVDIDIK

SEQ ID NO: 302 - protein, 11 aa, Homo sapiens
MERNSELQALR

SEQ ID NO: 303 - protein, 12 aa, Homo sapiens
QEINSHVEMQTK

SEQ ID NO: 304 - protein, 16 aa, Homo sapiens
MPVDPPGKPEVIDVTK

SEQ ID NO: 305 - protein, 10 aa, Homo sapiens
DQQPVTAGAR

SEQ ID NO: 306 - protein, 18 aa, Homo sapiens
TILGTMPAFEVSLQALQK

SEQ ID NO: 307 - protein, 20 aa, Homo sapiens
CPAPELLGGPSVFLFPPKPK

SEQ ID NO: 308 - protein, 11 aa, Homo sapiens
HVVPNEVVVQR

SEQ ID NO: 309 - protein, 7 aa, Homo sapiens
YQISVNK

SEQ ID NO: 310 - protein, 17 aa, Homo sapiens
QPFVQGLALYTPVVLPR

SEQ ID NO: 311 - protein, 6 aa, Homo sapiens
EKGANK

SEQ ID NO: 312 - protein, 7 aa, Homo sapiens
AMAELAA

SEQ ID NO: 313 - protein, 9 aa, Homo sapiens
VVNVANVGA

SEQ ID NO: 314 - protein, 11 aa, Homo sapiens
ENADSLQASLR

SEQ ID NO: 315 - protein, 9 aa, Homo sapiens
ATVLNYLPK

SEQ ID NO: 316 - protein, 7 aa, Homo sapiens
SCAVAEY

SEQ ID NO: 317 - protein, 9 aa, Homo sapiens
CFQGNQFLR

SEQ ID NO: 318 - protein, 6 aa, Homo sapiens
FSVSLK

SEQ ID NO: 319 - protein, 16 aa, Homo sapiens
TNQANFSRITQDAQLK

SEQ ID NO: 320 - protein, 17 aa, Homo sapiens
VVLVSLQSGYLFIQTDK

SEQ ID NO: 321 - protein, 7 aa, Homo sapiens
VLGQVNK

SEQ ID NO: 322 - protein, 10 aa, Homo sapiens
VLVDHFGYTK

SEQ ID NO: 323 - protein, 15 aa, Homo sapiens
LDLQEINNWVQAQMK

SEQ ID NO: 324 - protein, 22 aa, Homo sapiens
ELGCGAASGTPSGILYEPPAEK

SEQ ID NO: 325 - protein, 18 aa, Homo sapiens
SLGECCDVEDSTTCFNAK

SEQ ID NO: 326 - protein, 14 aa, Homo sapiens
VPFDAATLHTSTAM

SEQ ID NO: 327 - protein, 21 aa, Homo sapiens
CSPHLVLSALTSDNHGATYAF

SEQ ID NO: 328 - protein, 12 aa, Homo sapiens
AYLEEECPATLR

SEQ ID NO: 329 - protein, 9 aa, Homo sapiens
EIGELYLPK

SEQ ID NO: 330 - protein, 17 aa, Homo sapiens
SSNNPHSPIVEEFQVPY

SEQ ID NO: 331 - protein, 19 aa, Homo sapiens
VVNNSPQPQNVVFDVQIPK

SEQ ID NO: 332 - protein, 11 aa, Homo sapiens
GSESGIFTNTK

SEQ ID NO: 333 - protein, 6 aa, Homo sapiens
LPGVNM

SEQ ID NO: 334 - protein, 14 aa, Homo sapiens
MAVQLLDSSNQEER

SEQ ID NO: 335 - protein, 17 aa, Homo sapiens
GQELCADYSENTFTEYK

SEQ ID NO: 336 - protein, 9 aa, Homo sapiens
QKPLSITVR

SEQ ID NO: 337 - protein, 9 aa, Homo sapiens
GFVVAGPSR

SEQ ID NO: 338 - protein, 10 aa, Homo sapiens
EGDCPVQSGK

SEQ ID NO: 339 - protein, 8 aa, Homo sapiens
GEFYIGSK

SEQ ID NO: 340 - protein, 12 aa, Homo sapiens
MYLGYEYVTAIR

SEQ ID NO: 341 - protein, 9 aa, Homo sapiens
DRVNKQFVG

SEQ ID NO: 342 - protein, 8 aa, Homo sapiens
GYVIIKPL

SEQ ID NO: 343 - protein, 7 aa, Homo sapiens
AVLYLER

SEQ ID NO: 344 - protein, 13 aa, Homo sapiens
SLCSDQQSHLEFR

SEQ ID NO: 345 - protein, 9 aa, Homo sapiens
LEEQAQQIR

SEQ ID NO: 346 - protein, 23 aa, Homo sapiens
MATKAVCVLKGDGPVQGIINFEQ

SEQ ID NO: 347 - protein, 13 aa, Homo sapiens
SPDGDSSLAASER

SEQ ID NO: 348 - protein, 7 aa, Homo sapiens
AEGGLIK

SEQ ID NO: 349 - protein, 23 aa, Homo sapiens
FPLENTELANVGANQVTVRKGEK

SEQ ID NO: 350 - protein, 23 aa, Homo sapiens
LLNNWDVCADMVGTFTDTEDPAK

SEQ ID NO: 351 - protein, 26 aa, Homo sapiens
QKQQNFQAELDASMHQQQELQREGQR

SEQ ID NO: 352 - protein, 8 aa, Homo sapiens
YFIDFVAR

SEQ ID NO: 353 - protein, 6 aa, Homo sapiens
MMQEQI

SEQ ID NO: 354 - protein, 19 aa, Homo sapiens
QCENPNMECILFHVVAIGR

SEQ ID NO: 355 - protein, 7 aa, Homo sapiens
MAIVDVK

SEQ ID NO: 356 - protein, 7 aa, Homo sapiens
YIASAFR

SEQ ID NO: 357 - protein, 20 aa, Homo sapiens
GHCTKMATRGSNGTICDNQR

SEQ ID NO: 358 - protein, 16 aa, Homo sapiens
MVETTAYALLTSLNLK

SEQ ID NO: 359 - protein, 13 aa, Homo sapiens
MKPVPDLVPGNFK

SEQ ID NO: 360 - protein, 7 aa, Homo sapiens
CNLLAEK

SEQ ID NO: 361 - protein, 6 aa, Homo sapiens
EIEEIR

SEQ ID NO: 362 - protein, 16 aa, Homo sapiens
LVGGPMDASVEEEGVR

SEQ ID NO: 363 - protein, 16 aa, Homo sapiens
NIMEILRGDFSSANNR

SEQ ID NO: 364 - protein, 18 aa, Homo sapiens
SELTQDPAVSVALGQTVR

SEQ ID NO: 365 - protein, 10 aa, Homo sapiens
QSLLDDANLR

SEQ ID NO: 366 - protein, 10 aa, Homo sapiens
LSINTHPSQK

SEQ ID NO: 367 - protein, 10 aa, Homo sapiens
KQGGAAKDTR

SEQ ID NO: 368 - protein, 7 aa, Homo sapiens
GVISIPR

SEQ ID NO: 369 - protein, 6 aa, Homo sapiens
TTFDPK

SEQ ID NO: 370 - protein, 10 aa, Homo sapiens
QGIPFFGQVR

SEQ ID NO: 371 - protein, 15 aa, Homo sapiens
SGSSTASWIQNVDTK

SEQ ID NO: 372 - protein, 8 aa, Homo sapiens
CAGPAYLK

SEQ ID NO: 373 - protein, 16 aa, Homo sapiens
SGTASVVCLLNNFYPR

SEQ ID NO: 374 - protein, 31 aa, Homo sapiens
VQSTELCAGHLAGGTDSCQGDSGGPLVCFEK

SEQ ID NO: 375 - protein, 12 aa, Homo sapiens
SKAFSVVIQASK

SEQ ID NO: 376 - protein, 11 aa, Homo sapiens
LPPNVVEESAR

SEQ ID NO: 377 - protein, 21 aa, Homo sapiens
PVAEANAASMCLLANVAHANR

SEQ ID NO: 378 - protein, 16 aa, Homo sapiens
VQEAHLTEDQIFYFPK

SEQ ID NO: 379 - protein, 6 aa, Homo sapiens
NFNAIR

SEQ ID NO: 380 - protein, 7 aa, Homo sapiens
TESIIHR

SEQ ID NO: 381 - protein, 9 aa, Homo sapiens
YQIAQAEAK

SEQ ID NO: 382 - protein, 11 aa, Homo sapiens
MALAMAKPMAK

SEQ ID NO: 383 - protein, 18 aa, Homo sapiens
NPDNDPQGPWCYTTDPEK

SEQ ID NO: 384 - protein, 14 aa, Homo sapiens
IPIEDGSGEVVLSR

SEQ ID NO: 385 - protein, 11 aa, Homo sapiens
STTAPGTPSSN

SEQ ID NO: 386 - protein, 12 aa, Homo sapiens
IDTQDIEASHYR

SEQ ID NO: 387 - protein, 7 aa, Homo sapiens
VVNPTEA

SEQ ID NO: 388 - protein, 19 aa, Homo sapiens
LNLVATPLFLKPGIPYPIK

SEQ ID NO: 389 - protein, 9 aa, Homo sapiens
TSQEDLVEK

SEQ ID NO: 390 - protein, 18 aa, Homo sapiens
LLNLDGTCADSYSFVFSR

SEQ ID NO: 391 - protein, 11 aa, Homo sapiens
KGEWVALNPLR

SEQ ID NO: 392 - protein, 8 aa, Homo sapiens
KTNIEVEK

SEQ ID NO: 393 - protein, 6 aa, Homo sapiens
EVHLVQ

SEQ ID NO: 394 - protein, 11 aa, Homo sapiens
GYVIIKPLVWV

SEQ ID NO: 395 - protein, 29 aa, Homo sapiens
QQMMQQQQQQGAGPGIGPGMANHNQFQQP

SEQ ID NO: 396 - protein, 9 aa, Homo sapiens
CLAPLEGAR

SEQ ID NO: 397 - protein, 6 aa, Homo sapiens
YGAAMV

SEQ ID NO: 398 - protein, 9 aa, Homo sapiens
NDCQQMIVK

SEQ ID NO: 399 - protein, 11 aa, Homo sapiens
DIFTGLIGPMK

SEQ ID NO: 400 - protein, 22 aa, Homo sapiens
DCDKMDKACMGGLPSNAYSAIK

SEQ ID NO: 401 - protein, 8 aa, Homo sapiens
FPVEMTHN

SEQ ID NO: 402 - protein, 16 aa, Homo sapiens
DICEEQVNSLPGSITK

SEQ ID NO: 403 - protein, 6 aa, Homo sapiens
NWIQYK

SEQ ID NO: 404 - protein, 13 aa, Homo sapiens
DVMQGTDEHVVCK

SEQ ID NO: 405 - protein, 12 aa, Homo sapiens
SMCVPDQDTAIR

SEQ ID NO: 406 - protein, 14 aa, Homo sapiens
GDATVFFILPNQGK

SEQ ID NO: 407 - protein, 22 aa, Homo sapiens
ASNAWILQQHIATVPSLTHLCR

SEQ ID NO: 408 - protein, 16 aa, Homo sapiens
EYVLPSFEVIVEPTEK

SEQ ID NO: 409 - protein, 7 aa, Homo sapiens
LSAQMKA

SEQ ID NO: 410 - protein, 13 aa, Homo sapiens
AADDTWEPFASGK

SEQ ID NO: 411 - protein, 17 aa, Homo sapiens
KDTVIKPLLVEPEGLEK

SEQ ID NO: 412 - protein, 7 aa, Homo sapiens
IALLQLK

SEQ ID NO: 413 - protein, 12 aa, Homo sapiens
NTLELSNGVIVK

SEQ ID NO: 414 - protein, 15 aa, Homo sapiens
SPQELLCGASLISDR

SEQ ID NO: 415 - protein, 6 aa, Homo sapiens
VSQQNK

SEQ ID NO: 416 - protein, 19 aa, Homo sapiens
EANVACLDLGFQQGADTQR

SEQ ID NO: 417 - protein, 22 aa, Homo sapiens
RNSEVKYTEACSSSSVESGIVN

SEQ ID NO: 418 - protein, 12 aa, Homo sapiens
NVQDAIADAEQR

SEQ ID NO: 419 - protein, 6 aa, Homo sapiens
SSEDII

SEQ ID NO: 420 - protein, 19 aa, Homo sapiens
EGNVRKVVATTQMQAADAR

SEQ ID NO: 421 - protein, 20 aa, Homo sapiens
DGSPDVTTADIGANTPDATK

SEQ ID NO: 422 - protein, 7 aa, Homo sapiens
VGFYESD

SEQ ID NO: 423 - protein, 19 aa, Homo sapiens
GFPIKEDFLEQSEQLFGAK

SEQ ID NO: 424 - protein, 10 aa, Homo sapiens
LALDNGGLAR

SEQ ID NO: 425 - protein, 17 aa, Homo sapiens
YGFCEAADQFHVLDEVR

SEQ ID NO: 426 - protein, 13 aa, Homo sapiens
GLIDEVNQDFTNR

SEQ ID NO: 427 - protein, 12 aa, Homo sapiens
DEIPHNDIALLK

SEQ ID NO: 428 - protein, 11 aa, Homo sapiens
LVPFATELHER

SEQ ID NO: 429 - protein, 15 aa, Homo sapiens
FTCTVTHTDLPSPLK

SEQ ID NO: 430 - protein, 14 aa, Homo sapiens
APGSVAKMESNLSR

SEQ ID NO: 431 - protein, 7 aa, Homo sapiens
LQEVELR

SEQ ID NO: 432 - protein, 15 aa, Homo sapiens
ANTVQEATFQMELPK

SEQ ID NO: 433 - protein, 9 aa, Homo sapiens
YNLTLIWNR

SEQ ID NO: 434 - protein, 6 aa, Homo sapiens
VSEDLR

SEQ ID NO: 435 - protein, 10 aa, Homo sapiens
NMQDMVEDYR

SEQ ID NO: 436 - protein, 7 aa, Homo sapiens
DSLQQSS

SEQ ID NO: 437 - protein, 13 aa, Homo sapiens
VNMAITEHPENFK

SEQ ID NO: 438 - protein, 14 aa, Homo sapiens
AGEQVTYTCATYYK

SEQ ID NO: 439 - protein, 13 aa, Homo sapiens
DEAKSLIERYGGK

SEQ ID NO: 440 - protein, 21 aa, Homo sapiens
RQECSIPVCGQDQVTVAMTPR

SEQ ID NO: 441 - protein, 6 aa, Homo sapiens
MPCPGR

SEQ ID NO: 442 - protein, 16 aa, Homo sapiens
QSQMAEKQLEESVSEK

SEQ ID NO: 443 - protein, 10 aa, Homo sapiens
FPLYNLGFGH

SEQ ID NO: 444 - protein, 9 aa, Homo sapiens
IDFLNNYAL

SEQ ID NO: 445 - protein, 17 aa, Homo sapiens
AQTIDVNQETSDLDPSK

SEQ ID NO: 446 - protein, 11 aa, Homo sapiens
EDPFSINSGKK

SEQ ID NO: 447 - protein, 16 aa, Homo sapiens
IFFYDSENPPASEVLR

SEQ ID NO: 448 - protein, 16 aa, Homo sapiens
CQPVDCGIPESIENGK

SEQ ID NO: 449 - protein, 22 aa, Homo sapiens
NANQQKESMEQFIVSQLTRTHD

SEQ ID NO: 450 - protein, 12 aa, Homo sapiens
GFEPTLEALFGK

SEQ ID NO: 451 - protein, 15 aa, Homo sapiens
ENKDENHLPLVPPNK

SEQ ID NO: 452 - protein, 20 aa, Homo sapiens
SCESNSPFPVHPGTAECCTK

SEQ ID NO: 453 - protein, 9 aa, Homo sapiens
YTEFTPTEK

SEQ ID NO: 454 - protein, 9 aa, Homo sapiens
SAALSASYK

SEQ ID NO: 455 - protein, 6 aa, Homo sapiens
TETSLP

SEQ ID NO: 456 - protein, 28 aa, Homo sapiens
DGWHSWPIAHQWPQGPSAVDAAFSWEEK

SEQ ID NO: 457 - protein, 7 aa, Homo sapiens
WEYCNLK

SEQ ID NO: 458 - protein, 16 aa, Homo sapiens
LSNNALSGLPQGVFGK

SEQ ID NO: 459 - protein, 16 aa, Homo sapiens
SCEVVSVCLPLNLDTK

SEQ ID NO: 460 - protein, 8 aa, Homo sapiens
FLQEQGHR

SEQ ID NO: 461 - protein, 8 aa, Homo sapiens
QLEQVIAK

SEQ ID NO: 462 - protein, 9 aa, Homo sapiens
LPVSLSEGR

SEQ ID NO: 463 - protein, 16 aa, Homo sapiens
EKGVNYTQLSIDNCDR

SEQ ID NO: 464 - protein, 18 aa, Homo sapiens
TPSAAYLWVGTGASEAEK

SEQ ID NO: 465 - protein, 7 aa, Homo sapiens
TVVNVRN

SEQ ID NO: 466 - protein, 10 aa, Homo sapiens
EELLPAQDIK

SEQ ID NO: 467 - protein, 12 aa, Homo sapiens
TIYTPGSTVLYR

SEQ ID NO: 468 - protein, 9 aa, Homo sapiens
FMQAVTGWK

SEQ ID NO: 469 - protein, 22 aa, Homo sapiens
EPGQDLVVLPLSITTDFIPSFR

SEQ ID NO: 470 - protein, 8 aa, Homo sapiens
YTPPDCTP

SEQ ID NO: 471 - protein, 10 aa, Homo sapiens
QGHNSVFLIK

SEQ ID NO: 472 - protein, 20 aa, Homo sapiens
KEDSCQLGYSAGPCMGMTSR

SEQ ID NO: 473 - protein, 13 aa, Homo sapiens
CQFFSYATQTFHK

SEQ ID NO: 474 - protein, 21 aa, Homo sapiens
AGTELVNFLSYFVELGTQPAT

SEQ ID NO: 475 - protein, 10 aa, Homo sapiens
LDFSSQADLR

SEQ ID NO: 476 - protein, 7 aa, Homo sapiens
LSSQGAR

SEQ ID NO: 477 - protein, 14 aa, Homo sapiens
TQQPGMEQLSLMAG

SEQ ID NO: 478 - protein, 35 aa, Homo sapiens
QPADKASASGSGAQVGGPISSGSSASSVTVTRSYR

SEQ ID NO: 479 - protein, 19 aa, Homo sapiens
QLYNVEATSYALLALLQLK

SEQ ID NO: 480 - protein, 7 aa, Homo sapiens
ITLPDFR

SEQ ID NO: 481 - protein, 12 aa, Homo sapiens
TNQVNSGGVLLR

SEQ ID NO: 482 - protein, 18 aa, Homo sapiens
GIILDSVDAAFICPGSSR

SEQ ID NO: 483 - protein, 13 aa, Homo sapiens
TLADLTLLDSPIK

SEQ ID NO: 484 - protein, 9 aa, Homo sapiens
TVEEFVLPK

SEQ ID NO: 485 - protein, 17 aa, Homo sapiens
SQCLEDHTWAPPFPICK

SEQ ID NO: 486 - protein, 6 aa, Homo sapiens
MMNDQL

SEQ ID NO: 487 - protein, 13 aa, Homo sapiens
ENADSLQASLRPH

SEQ ID NO: 488 - protein, 17 aa, Homo sapiens
LAPNNLKPVVAEFYGSK

SEQ ID NO: 489 - protein, 18 aa, Homo sapiens
LYGSEAFATDFQDSAAAK

SEQ ID NO: 490 - protein, 16 aa, Homo sapiens
AMGSVMAPLVQAAFHR

SEQ ID NO: 491 - protein, 9 aa, Homo sapiens
KGGETSEMY

SEQ ID NO: 492 - protein, 17 aa, Homo sapiens
QESQSEEIDCNDKDLFK

SEQ ID NO: 493 - protein, 24 aa, Homo sapiens
ASQPSSFHDFPDLGQEVALNANTK

SEQ ID NO: 494 - protein, 8 aa, Homo sapiens
CLVNLIEK

SEQ ID NO: 495 - protein, 9 aa, Homo sapiens
IYISGMAPR

SEQ ID NO: 496 - protein, 7 aa, Homo sapiens
FNRPFLM

SEQ ID NO: 497 - protein, 20 aa, Homo sapiens
GRPGPQPWCATTPNFDQDQR

SEQ ID NO: 498 - protein, 11 aa, Homo sapiens
DSCTLPASAEK

SEQ ID NO: 499 - protein, 27 aa, Homo sapiens
LQEDADGSCALLSPYVQPVCLPSGAAR

SEQ ID NO: 500 - protein, 23 aa, Homo sapiens
TKQLFPGGTFPEDFSILFTVKPK

SEQ ID NO: 501 - protein, 6 aa, Homo sapiens
QQLKPY

SEQ ID NO: 502 - protein, 14 aa, Homo sapiens
EQLGEFYEALDCLR

SEQ ID NO: 503 - protein, 14 aa, Homo sapiens
AVLPTGDVIGDSAK

SEQ ID NO: 504 - protein, 7 aa, Homo sapiens
MFPEQQK

SEQ ID NO: 505 - protein, 7 aa, Homo sapiens
ECEEIIR

SEQ ID NO: 506 - protein, 10 aa, Homo sapiens
HQQTVTIPPK

SEQ ID NO: 507 - protein, 8 aa, Homo sapiens
GLVLVPTK

SEQ ID NO: 508 - protein, 11 aa, Homo sapiens
CDEWSVNSVGK

SEQ ID NO: 509 - protein, 6 aa, Homo sapiens
FGCFLK

SEQ ID NO: 510 - protein, 20 aa, Homo sapiens
TFYEPGEEITYSCKPGYVSR

SEQ ID NO: 511 - protein, 14 aa, Homo sapiens
DLHFLEELQLGHNR

SEQ ID NO: 512 - protein, 13 aa, Homo sapiens
VAQLEAQCQEPCK

SEQ ID NO: 513 - protein, 15 aa, Homo sapiens
AIQLTYNPDESSKPN

SEQ ID NO: 514 - protein, 21 aa, Homo sapiens
TIQQLLEQTVSASDADQQALR

SEQ ID NO: 515 - protein, 12 aa, Homo sapiens
AVFQDEGAEPLK

SEQ ID NO: 516 - protein, 13 aa, Homo sapiens
YPTPGEAPGVVGN

SEQ ID NO: 517 - protein, 8 aa, Homo sapiens
PAGLPEKY

SEQ ID NO: 518 - protein, 16 aa, Homo sapiens
LAGLGLQQLDEGLFSR

SEQ ID NO: 519 - protein, 14 aa, Homo sapiens
LGNVQLPQAPMGPR

SEQ ID NO: 520 - protein, 8 aa, Homo sapiens
NSLFEYQK

SEQ ID NO: 521 - protein, 10 aa, Homo sapiens
NALALFVLPK

SEQ ID NO: 522 - protein, 9 aa, Homo sapiens
VEGLTFQQN

SEQ ID NO: 523 - protein, 13 aa, Homo sapiens
ITCTEEGWSPTPK

SEQ ID NO: 524 - protein, 20 aa, Homo sapiens
CLPVFENAAFGRLAQASHTC

SEQ ID NO: 525 - protein, 12 aa, Homo sapiens
CCESASEDCMAK

SEQ ID NO: 526 - protein, 12 aa, Homo sapiens
HQFLLTGDTQGR

SEQ ID NO: 527 - protein, 11 aa, Homo sapiens
SVQEIQATFFY

SEQ ID NO: 528 - protein, 12 aa, Homo sapiens
SSNLIILEEHLK

SEQ ID NO: 529 - protein, 7 aa, Homo sapiens
IAQWQSF

SEQ ID NO: 530 - protein, 9 aa, Homo sapiens
DGNTLTYYR

SEQ ID NO: 531 - protein, 12 aa, Homo sapiens
VGWYPEPQVQWR

SEQ ID NO: 532 - protein, 16 aa, Homo sapiens
SNALDIIFQTDLTGQK

SEQ ID NO: 533 - protein, 9 aa, Homo sapiens
QGSFQGGFR

SEQ ID NO: 534 - protein, 7 aa, Homo sapiens
RYQAGMR

SEQ ID NO: 535 - protein, 22 aa, Homo sapiens
SNFLNCYVSGFHPSDIEVDLLK

SEQ ID NO: 536 - protein, 15 aa, Homo sapiens
ADSPMDDFFQCVNGK

SEQ ID NO: 537 - protein, 12 aa, Homo sapiens
MTGFAPDTDDLK

SEQ ID NO: 538 - protein, 9 aa, Homo sapiens
NGPLSCGQR

SEQ ID NO: 539 - protein, 11 aa, Homo sapiens
SEDTAVYYCAR

SEQ ID NO: 540 - protein, 19 aa, Homo sapiens
ILGEELGFASLHDLQLLGK

SEQ ID NO: 541 - protein, 7 aa, Homo sapiens
MNKNEAI

SEQ ID NO: 542 - protein, 7 aa, Homo sapiens
MEEVEAM

SEQ ID NO: 543 - protein, 19 aa, Homo sapiens
EPLDDYVNTQGASLFSVTK

SEQ ID NO: 544 - protein, 13 aa, Homo sapiens
KTSLEDFYLDEER

SEQ ID NO: 545 - protein, 14 aa, Homo sapiens
IASFSQNCDIYPGK

SEQ ID NO: 546 - protein, 8 aa, Homo sapiens
TETGGQGK

SEQ ID NO: 547 - protein, 12 aa, Homo sapiens
YAATSQVLLPSK

SEQ ID NO: 548 - protein, 11 aa, Homo sapiens
RKDSGSPNPAR

SEQ ID NO: 549 - protein, 21 aa, Homo sapiens
NIPTVNENLENYYLEVNQLEK

SEQ ID NO: 550 - protein, 20 aa, Homo sapiens
LTAQPAPTSEDLTSATNIVK

SEQ ID NO: 551 - protein, 12 aa, Homo sapiens
GFMQTYYDDHLR

SEQ ID NO: 552 - protein, 8 aa, Homo sapiens
TTLTAFGF

SEQ ID NO: 553 - protein, 12 aa, Homo sapiens
TDVNYQFTQIVV

SEQ ID NO: 554 - protein, 32 aa, Homo sapiens
FDVSSFNPHGISTFTDEDNAMYLLVVNHPDAK

SEQ ID NO: 555 - protein, 21 aa, Homo sapiens
KGDTFSCMVGHEALPLAFTQK

SEQ ID NO: 556 - protein, 22 aa, Homo sapiens
VPVAVQGEDTVQSLTQGDGVAK

SEQ ID NO: 557 - protein, 6 aa, Homo sapiens
VLQNAR

SEQ ID NO: 558 - protein, 13 aa, Homo sapiens
LESDVSAQMEYCR

SEQ ID NO: 559 - protein, 9 aa, Homo sapiens
LLIYGASSR

SEQ ID NO: 560 - protein, 21 aa, Homo sapiens
SLWNGDTGECTDNAYIDIQLR

SEQ ID NO: 561 - protein, 19 aa, Homo sapiens
LSIFLAWDVDIGSDNTDSR

SEQ ID NO: 562 - protein, 22 aa, Homo sapiens
VLLSPLSVATALSALSLGAEQR

SEQ ID NO: 563 - protein, 9 aa, Homo sapiens
CLHPCVISR

SEQ ID NO: 564 - protein, 12 aa, Homo sapiens
EPPYFVEKPQSQ

SEQ ID NO: 565 - protein, 9 aa, Homo sapiens
ILIGTVFHK

SEQ ID NO: 566 - protein, 11 aa, Homo sapiens
LPVADQDQCIR

SEQ ID NO: 567 - protein, 10 aa, Homo sapiens
ALQEWLLKQK

SEQ ID NO: 568 - protein, 9 aa, Homo sapiens
ALTDMPQMR

SEQ ID NO: 569 - protein, 11 aa, Homo sapiens
TVLHQDWLNGK

SEQ ID NO: 570 - protein, 18 aa, Homo sapiens
KVEGTAFVIFGIQDGEQR

SEQ ID NO: 571 - protein, 19 aa, Homo sapiens
KCSYTEDAQCIDGTIEVPK

SEQ ID NO: 572 - protein, 8 aa, Homo sapiens
EQYAVVGH

SEQ ID NO: 573 - protein, 11 aa, Homo sapiens
EQQALQTVCLK

SEQ ID NO: 574 - protein, 6 aa, Homo sapiens
AELQER

SEQ ID NO: 575 - protein, 9 aa, Homo sapiens
SNVLVFQQP

SEQ ID NO: 576 - protein, 6 aa, Homo sapiens
LEADYD

SEQ ID NO: 577 - protein, 6 aa, Homo sapiens
YGAATF

SEQ ID NO: 578 - protein, 7 aa, Homo sapiens
IWDVVEK

SEQ ID NO: 579 - protein, 6 aa, Homo sapiens
EELELR

SEQ ID NO: 580 - protein, 8 aa, Homo sapiens
VYPGEQYT

SEQ ID NO: 581 - protein, 7 aa, Homo sapiens
NSLNPFK

SEQ ID NO: 582 - protein, 9 aa, Homo sapiens
DSVTGTLPK

SEQ ID NO: 583 - protein, 15 aa, Homo sapiens
HLEVDVWVIEPQGLR

SEQ ID NO: 584 - protein, 19 aa, Homo sapiens
AFPALTSLDLSDNPGLGER

SEQ ID NO: 585 - protein, 14 aa, Homo sapiens
WEMPFDPQDTHQSR

SEQ ID NO: 586 - protein, 20 aa, Homo sapiens
ILLQGTPVAQMTEDAVDAER

SEQ ID NO: 587 - protein, 11 aa, Homo sapiens
MNAKWDTGENP

SEQ ID NO: 588 - protein, 14 aa, Homo sapiens
LEQGENVFLQATDK

SEQ ID NO: 589 - protein, 14 aa, Homo sapiens
MVSVEELEHSISIK

SEQ ID NO: 590 - protein, 23 aa, Homo sapiens
WVQTLSEQVQEELLSSQVTQELR

SEQ ID NO: 591 - protein, 10 aa, Homo sapiens
DSGFQMNQLR

SEQ ID NO: 592 - protein, 14 aa, Homo sapiens
DSPVLIDFFEDTER

SEQ ID NO: 593 - protein, 13 aa, Homo sapiens
QEDDLANINQWVK

SEQ ID NO: 594 - protein, 17 aa, Homo sapiens
FEGNCAEQDGSGWWMNK

SEQ ID NO: 595 - protein, 11 aa, Homo sapiens
QQQQQQETSPR

SEQ ID NO: 596 - protein, 21 aa, Homo sapiens
RVNILANDNVAGIVSFQTASR

SEQ ID NO: 597 - protein, 12 aa, Homo sapiens
DTCGVGYVALGE

SEQ ID NO: 598 - protein, 27 aa, Homo sapiens
NLTGQSGQNAGNLIILKNEAVSQNEVR

SEQ ID NO: 599 - protein, 13 aa, Homo sapiens
ELQYLGQIQHILR

SEQ ID NO: 600 - protein, 9 aa, Homo sapiens
FPEVDVLTK

SEQ ID NO: 601 - protein, 12 aa, Homo sapiens
ASVSVLGDILGS

SEQ ID NO: 602 - protein, 20 aa, Homo sapiens
LVFQQFDLEPSEGCFYDYVK

SEQ ID NO: 603 - protein, 7 aa, Homo sapiens
SLDFSSK

SEQ ID NO: 604 - protein, 17 aa, Homo sapiens
VQAAVGTSAAPVPSDNH

SEQ ID NO: 605 - protein, 7 aa, Homo sapiens
LSLQEND

SEQ ID NO: 606 - protein, 15 aa, Homo sapiens
ALYWVNGQVPDGVSK

SEQ ID NO: 607 - protein, 6 aa, Homo sapiens
DIASGL

SEQ ID NO: 608 - protein, 10 aa, Homo sapiens
GQYCYELDEK

SEQ ID NO: 609 - protein, 13 aa, Homo sapiens
TEGDGVYTLNNEK

SEQ ID NO: 610 - protein, 9 aa, Homo sapiens
VVLGDQDLK

SEQ ID NO: 611 - protein, 7 aa, Homo sapiens
ALDLINK

SEQ ID NO: 612 - protein, 11 aa, Homo sapiens
HSTIFENLANK

SEQ ID NO: 613 - protein, 15 aa, Homo sapiens
ALLVGEHLNIIVTPK

SEQ ID NO: 614 - protein, 9 aa, Homo sapiens
YYCFQGNQF

SEQ ID NO: 615 - protein, 12 aa, Homo sapiens
FQSGQVLAALPR

SEQ ID NO: 616 - protein, 12 aa, Homo sapiens
QMFGNADMNTFP

SEQ ID NO: 617 - protein, 8 aa, Homo sapiens
GPLDQLEK

SEQ ID NO: 618 - protein, 19 aa, Homo sapiens
DIPTNSPELEETLTHTITK

SEQ ID NO: 619 - protein, 6 aa, Homo sapiens
IDGQFR

SEQ ID NO: 620 - protein, 9 aa, Homo sapiens
GNLCVNLMR

SEQ ID NO: 621 - protein, 6 aa, Homo sapiens
LKKAHK

SEQ ID NO: 622 - protein, 20 aa, Homo sapiens
SPTSYTECLNGAQSLHRKHK

SEQ ID NO: 623 - protein, 12 aa, Homo sapiens
INVEIKGEEKVA

SEQ ID NO: 624 - protein, 18 aa, Homo sapiens
ASVSVLGDILGSAMQNTQ

SEQ ID NO: 625 - protein, 8 aa, Homo sapiens
FATTFYQH

SEQ ID NO: 626 - protein, 7 aa, Homo sapiens
VVLVSLQ

SEQ ID NO: 627 - protein, 9 aa, Homo sapiens
DVCDPGNTK

SEQ ID NO: 628 - protein, 19 aa, Homo sapiens
IVSSAMEPDREYHFGQAVR

SEQ ID NO: 629 - protein, 20 aa, Homo sapiens
EPCVESLVSQYFQTVTDYGK

SEQ ID NO: 630 - protein, 7 aa, Homo sapiens
EEAPSLR

SEQ ID NO: 631 - protein, 17 aa, Homo sapiens
ELNHEKERCDQLQAEQK

SEQ ID NO: 632 - protein, 7 aa, Homo sapiens
VEVEAGK

SEQ ID NO: 633 - protein, 8 aa, Homo sapiens
QSLMMLQM

SEQ ID NO: 634 - protein, 8 aa, Homo sapiens
LEYLLLSR

SEQ ID NO: 635 - protein, 19 aa, Homo sapiens
EDSCQLGYSAGPCMGMTSR

SEQ ID NO: 636 - protein, 7 aa, Homo sapiens
DLGQCDR

SEQ ID NO: 637 - protein, 10 aa, Homo sapiens
VTSIQDWVQK

SEQ ID NO: 638 - protein, 9 aa, Homo sapiens
GWVDLFVPK

SEQ ID NO: 639 - protein, 8 aa, Homo sapiens
FDSVPTSR

SEQ ID NO: 640 - protein, 13 aa, Homo sapiens
ASADLIEIGLEGK

SEQ ID NO: 641 - protein, 20 aa, Homo sapiens
YEDGTLSLTSTSDLQSGIIK

SEQ ID NO: 642 - protein, 9 aa, Homo sapiens
NYNLVESLK

SEQ ID NO: 643 - protein, 12 aa, Homo sapiens
EEEQTAEEILSK

SEQ ID NO: 644 - protein, 8 aa, Homo sapiens
VYFAGFPR

SEQ ID NO: 645 - protein, 14 aa, Homo sapiens
WYNLAIGSTCPWLK

SEQ ID NO: 646 - protein, 11 aa, Homo sapiens
DTAVFECLPQH

SEQ ID NO: 647 - protein, 7 aa, Homo sapiens
DSGFQMN

SEQ ID NO: 648 - protein, 11 aa, Homo sapiens
DRDGNTLTYYR

SEQ ID NO: 649 - protein, 40 aa, Homo sapiens
LGEHNIDVLEGNEQFINAAKIITHPNFNGNTLDNDIMLIK

SEQ ID NO: 650 - protein, 7 aa, Homo sapiens
QGEGMVK

SEQ ID NO: 651 - protein, 7 aa, Homo sapiens
QVIITIL

SEQ ID NO: 652 - protein, 22 aa, Homo sapiens
NFVASHIANILNSEELDIQDLK

SEQ ID NO: 653 - protein, 16 aa, Homo sapiens
IEMNKADNDAGNAAID

SEQ ID NO: 654 - protein, 10 aa, Homo sapiens
TGESAEFVCK

SEQ ID NO: 655 - protein, 24 aa, Homo sapiens
LVLSALTSDNHGATYAFSGTHYWR

SEQ ID NO: 656 - protein, 6 aa, Homo sapiens
YPDNKV

SEQ ID NO: 657 - protein, 10 aa, Homo sapiens
SSPNGQGLQK

SEQ ID NO: 658 - protein, 12 aa, Homo sapiens
DCRVAQNGGFFR

SEQ ID NO: 659 - protein, 12 aa, Homo sapiens
ETAASLLQAGYK

SEQ ID NO: 660 - protein, 10 aa, Homo sapiens
AFALAGNQDK

SEQ ID NO: 661 - protein, 20 aa, Homo sapiens
TPGAAANLELIFVGPQHAGN

SEQ ID NO: 662 - protein, 6 aa, Homo sapiens
TTVMVK

SEQ ID NO: 663 - protein, 15 aa, Homo sapiens
YVTSAPMPEPQAPGR

SEQ ID NO: 664 - protein, 9 aa, Homo sapiens
FEVQVTVPK

SEQ ID NO: 665 - protein, 8 aa, Homo sapiens
DSAHGFLK

SEQ ID NO: 666 - protein, 9 aa, Homo sapiens
SIIFFLPLK

SEQ ID NO: 667 - protein, 15 aa, Homo sapiens
VIAVHYLDETEQWEK

SEQ ID NO: 668 - protein, 9 aa, Homo sapiens
KYFIDFVAR

SEQ ID NO: 669 - protein, 18 aa, Homo sapiens
LLIYAVLPTGDVIGDSAK

SEQ ID NO: 670 - protein, 8 aa, Homo sapiens
LAAEFASK

SEQ ID NO: 671 - protein, 18 aa, Homo sapiens
NPDADKGPWCFTTDPSVR

SEQ ID NO: 672 - protein, 19 aa, Homo sapiens
GSLVQASEANLQAAQDFVR

SEQ ID NO: 673 - protein, 12 aa, Homo sapiens
AGGSWDLAVQER

SEQ ID NO: 674 - protein, 6 aa, Homo sapiens
FWDYLR

SEQ ID NO: 675 - protein, 26 aa, Homo sapiens
PGFTIVGPNSVQCYHFGLSPDLPICK

SEQ ID NO: 676 - protein, 11 aa, Homo sapiens
HLSLLTTLSNR

SEQ ID NO: 677 - protein, 16 aa, Homo sapiens
NSPLDEENLTQENQDR

SEQ ID NO: 678 - protein, 15 aa, Homo sapiens
ITEVALMGHLSCDTK

SEQ ID NO: 679 - protein, 17 aa, Homo sapiens
TFHRAASSAAQGAFQGN

SEQ ID NO: 680 - protein, 37 aa, Homo sapiens
GSAGHWTSESSVSGSTGQWHSESGSFRPDSPGSGNAR

SEQ ID NO: 681 - protein, 7 aa, Homo sapiens
TSHQLQK

SEQ ID NO: 682 - protein, 15 aa, Homo sapiens
EYCGVPGDGDEELLR

SEQ ID NO: 683 - protein, 16 aa, Homo sapiens
FLSPSAQQASWQVSAR

SEQ ID NO: 684 - protein, 9 aa, Homo sapiens
TDASDVKPC

SEQ ID NO: 685 - protein, 19 aa, Homo sapiens
KVAMMTQPPATPTLPRLPH

SEQ ID NO: 686 - protein, 23 aa, Homo sapiens
PSQFGGQPCTEPLVAFQPCIPSK

SEQ ID NO: 687 - protein, 10 aa, Homo sapiens
NGMFFSTYDR

SEQ ID NO: 688 - protein, 11 aa, Homo sapiens
YNSQNQSNNQF

SEQ ID NO: 689 - protein, 9 aa, Homo sapiens
HADKGAGVK

SEQ ID NO: 690 - protein, 18 aa, Homo sapiens
LPSGLPVSLLTLYLDNNK

SEQ ID NO: 691 - protein, 14 aa, Homo sapiens
SPELEETLTHTITK

SEQ ID NO: 692 - protein, 7 aa, Homo sapiens
AIQLTYN

SEQ ID NO: 693 - protein, 7 aa, Homo sapiens
DGDPFAK

SEQ ID NO: 694 - protein, 17 aa, Homo sapiens
KSGQKTVLSNVQEELDR

SEQ ID NO: 695 - protein, 12 aa, Homo sapiens
EWFWDLATGTMK

SEQ ID NO: 696 - protein, 18 aa, Homo sapiens
SPVGVQPILNEHTFCVGM

SEQ ID NO: 697 - protein, 11 aa, Homo sapiens
CHAGHLNGVYY

SEQ ID NO: 698 - protein, 12 aa, Homo sapiens
FEDGVLDPDYPR

SEQ ID NO: 699 - protein, 14 aa, Homo sapiens
VEDPESTLFGSVIR

SEQ ID NO: 700 - protein, 11 aa, Homo sapiens
FKPENGLLETK

SEQ ID NO: 701 - protein, 16 aa, Homo sapiens
EPGPPIYAAPSNLKRA

SEQ ID NO: 702 - protein, 9 aa, Homo sapiens
NIQRNSINK

SEQ ID NO: 703 - protein, 8 aa, Homo sapiens
RTQNTLSK

SEQ ID NO: 704 - protein, 17 aa, Homo sapiens
VDFLLSVIGYAVDLGNV

SEQ ID NO: 705 - protein, 7 aa, Homo sapiens
DGVLEPK

SEQ ID NO: 706 - protein, 9 aa, Homo sapiens
CAGQQQDIR

SEQ ID NO: 707 - protein, 24 aa, Homo sapiens
VPGISQAVQQDSATAAETKEQEIS

SEQ ID NO: 708 - protein, 14 aa, Homo sapiens
AQLDVLEALFAKTR

SEQ ID NO: 709 - protein, 16 aa, Homo sapiens
TMGYQDFADVVCYTQK

SEQ ID NO: 710 - protein, 9 aa, Homo sapiens
SDVMYTDWK

SEQ ID NO: 711 - protein, 8 aa, Homo sapiens
LYQLQVPL

SEQ ID NO: 712 - protein, 14 aa, Homo sapiens
YVMLPVADQDQCIR

SEQ ID NO: 713 - protein, 6 aa, Homo sapiens
FSLDGK

SEQ ID NO: 714 - protein, 9 aa, Homo sapiens
TYLANGQTK

SEQ ID NO: 715 - protein, 7 aa, Homo sapiens
FQVQHEK

SEQ ID NO: 716 - protein, 8 aa, Homo sapiens
EQHLFLPF

SEQ ID NO: 717 - protein, 7 aa, Homo sapiens
GVFVLNK

SEQ ID NO: 718 - protein, 19 aa, Homo sapiens
TGLDSPTGIDFSDITANSF

SEQ ID NO: 719 - protein, 10 aa, Homo sapiens
SGVEVRLPND

SEQ ID NO: 720 - protein, 8 aa, Homo sapiens
IGVELTGR

SEQ ID NO: 721 - protein, 9 aa, Homo sapiens
LFQPLTHLK

SEQ ID NO: 722 - protein, 13 aa, Homo sapiens
SWPAVGNCSSALR

SEQ ID NO: 723 - protein, 8 aa, Homo sapiens
SFILPGEK

SEQ ID NO: 724 - protein, 21 aa, Homo sapiens
VFALILALMLSMTGADSHAKR

SEQ ID NO: 725 - protein, 16 aa, Homo sapiens
FSEGTAGDSLSLHSGR

SEQ ID NO: 726 - protein, 8 aa, Homo sapiens
ANDHDQSR

SEQ ID NO: 727 - protein, 8 aa, Homo sapiens
FYYIYNEK

SEQ ID NO: 728 - protein, 7 aa, Homo sapiens
FFGEGTK

SEQ ID NO: 729 - protein, 8 aa, Homo sapiens
TYCSEPEK

SEQ ID NO: 730 - protein, 13 aa, Homo sapiens
GFQQLLQELNQPR

SEQ ID NO: 731 - protein, 6 aa, Homo sapiens
VSVNER

SEQ ID NO: 732 - protein, 11 aa, Homo sapiens
SITTDFIPSFR

SEQ ID NO: 733 - protein, 9 aa, Homo sapiens
SSSSNLGAD

SEQ ID NO: 734 - protein, 6 aa, Homo sapiens
DLEEVK

SEQ ID NO: 735 - protein, 7 aa, Homo sapiens
GWVTDGF

SEQ ID NO: 736 - protein, 10 aa, Homo sapiens
LVNEVTEFAK

SEQ ID NO: 737 - protein, 7 aa, Homo sapiens
SILFLGK

SEQ ID NO: 738 - protein, 16 aa, Homo sapiens
VVSVLTVLHQNWLDGK

SEQ ID NO: 739 - protein, 7 aa, Homo sapiens
SAKGVLE

SEQ ID NO: 740 - protein, 16 aa, Homo sapiens
PVAFSDYIHPVCLPDR

SEQ ID NO: 741 - protein, 16 aa, Homo sapiens
ADLFYDVEALDLESPK

SEQ ID NO: 742 - protein, 16 aa, Homo sapiens
FSGSNSGNTATLTISR

SEQ ID NO: 743 - protein, 17 aa, Homo sapiens
SSDWQSTDATPTLTNSS

SEQ ID NO: 744 - protein, 15 aa, Homo sapiens
VGATSFYSTCQSNGK

SEQ ID NO: 745 - protein, 8 aa, Homo sapiens
MEMVAQLR

SEQ ID NO: 746 - protein, 21 aa, Homo sapiens
EFNLQNMGLPDFHIPENLFLK

SEQ ID NO: 747 - protein, 25 aa, Homo sapiens
AGELEVFNGYFVHFFAPDNLDPIPK

SEQ ID NO: 748 - protein, 7 aa, Homo sapiens
QTVEAMK

SEQ ID NO: 749 - protein, 22 aa, Homo sapiens
FLEISQLEGTDSGTYTCSATNK

SEQ ID NO: 750 - protein, 7 aa, Homo sapiens
LDVMYSR

SEQ ID NO: 751 - protein, 15 aa, Homo sapiens
IIVPTDTQNIFFMSK

SEQ ID NO: 752 - protein, 6 aa, Homo sapiens
LSGRQK

SEQ ID NO: 753 - protein, 15 aa, Homo sapiens
YQMESFSKYSSVQKA

SEQ ID NO: 754 - protein, 9 aa, Homo sapiens
MATTMIQSK

SEQ ID NO: 755 - protein, 11 aa, Homo sapiens
IAPANADFAFR

SEQ ID NO: 756 - protein, 14 aa, Homo sapiens
TPECPSHTQPLGVY

SEQ ID NO: 757 - protein, 18 aa, Homo sapiens
DDKHEQDMVNGIMLSVEK

SEQ ID NO: 758 - protein, 16 aa, Homo sapiens
NSSNLMQKSPNVENPR

SEQ ID NO: 759 - protein, 27 aa, Homo sapiens
FSDEGTHESQISFTIEGPLTSFGLSNK

SEQ ID NO: 760 - protein, 15 aa, Homo sapiens
NIQEYLSILTDPDGK

SEQ ID NO: 761 - protein, 8 aa, Homo sapiens
AEQEKASF

SEQ ID NO: 762 - protein, 15 aa, Homo sapiens
YLVGRNNANGVDLNR

SEQ ID NO: 763 - protein, 14 aa, Homo sapiens
DLSASMDDDLNTIK

SEQ ID NO: 764 - protein, 15 aa, Homo sapiens
AATVGSLAGQPLQER

SEQ ID NO: 765 - protein, 10 aa, Homo sapiens
SSSHLSPSEE

SEQ ID NO: 766 - protein, 11 aa, Homo sapiens
FAPEGLTTMPK

SEQ ID NO: 767 - protein, 19 aa, Homo sapiens
SAVQNKVVVITDAISGLGK

SEQ ID NO: 768 - protein, 19 aa, Homo sapiens
EYKMGFGNPSGEYWLGNEF

SEQ ID NO: 769 - protein, 8 aa, Homo sapiens
DVAVGAPK

SEQ ID NO: 770 - protein, 7 aa, Homo sapiens
DVAVLCR

SEQ ID NO: 771 - protein, 10 aa, Homo sapiens
LPECEAVCGK

SEQ ID NO: 772 - protein, 14 aa, Homo sapiens
EHAVEGDCDFQLLK

SEQ ID NO: 773 - protein, 7 aa, Homo sapiens
MLGDVLM

SEQ ID NO: 774 - protein, 11 aa, Homo sapiens
GEDKMDGAPSR

SEQ ID NO: 775 - protein, 16 aa, Homo sapiens
DTVIKPLLVEPEGLEK

SEQ ID NO: 776 - protein, 19 aa, Homo sapiens
IFFHLNAVALGDGGHYTCR

SEQ ID NO: 777 - protein, 9 aa, Homo sapiens
PNVVEESAR

SEQ ID NO: 778 - protein, 15 aa, Homo sapiens
GDDITMVLILPKPEK

SEQ ID NO: 779 - protein, 11 aa, Homo sapiens
NAELALFELSR

SEQ ID NO: 780 - protein, 10 aa, Homo sapiens
PPNVQLTGYR

SEQ ID NO: 781 - protein, 25 aa, Homo sapiens
AQSTIIEIVSVQVPAQTPASPAKDK

SEQ ID NO: 782 - protein, 19 aa, Homo sapiens
VVAEGFDFANGINISPDGK

SEQ ID NO: 783 - protein, 26 aa, Homo sapiens
YGGGFYSTQDTINAIEGLTEYSLLVK

SEQ ID NO: 784 - protein, 10 aa, Homo sapiens
SSEPFQGSYK

SEQ ID NO: 785 - protein, 12 aa, Homo sapiens
YKGFNPSETKIR

SEQ ID NO: 786 - protein, 8 aa, Homo sapiens
TEDTIFLR

SEQ ID NO: 787 - protein, 7 aa, Homo sapiens
YEFLNGR

SEQ ID NO: 788 - protein, 19 aa, Homo sapiens
MIPPGTQLVKPKSEPQPNK

SEQ ID NO: 789 - protein, 8 aa, Homo sapiens
ELSSFIDK

SEQ ID NO: 790 - protein, 9 aa, Homo sapiens
QEKGTIDVW

SEQ ID NO: 791 - protein, 37 aa, Homo sapiens
LHDNQNGWSGDSAPVELILSDETLPAPEFSPEPESGR

SEQ ID NO: 792 - protein, 14 aa, Homo sapiens
SVIPSDGPSVACVK

SEQ ID NO: 793 - protein, 18 aa, Homo sapiens
VPTADLEDVLPLAEDITN

SEQ ID NO: 794 - protein, 6 aa, Homo sapiens
FSTYDR

SEQ ID NO: 795 - protein, 9 aa, Homo sapiens
WFYIASAFR

SEQ ID NO: 796 - protein, 13 aa, Homo sapiens
YYAGGCSPHYILN

SEQ ID NO: 797 - protein, 17 aa, Homo sapiens
MCPQLQQYEMHGPEGLR

SEQ ID NO: 798 - protein, 15 aa, Homo sapiens
YVVTSQVVNTANEAR

SEQ ID NO: 799 - protein, 13 aa, Homo sapiens
KGVCEETSGAYEK

SEQ ID NO: 800 - protein, 24 aa, Homo sapiens
PLLVDVDLQYPQDAVLALTQNHHK

SEQ ID NO: 801 - protein, 15 aa, Homo sapiens
LALSTAAQAEPVQFK

SEQ ID NO: 802 - protein, 13 aa, Homo sapiens
NLKPVVAEFYGSK

SEQ ID NO: 803 - protein, 18 aa, Homo sapiens
SAGSWNSGSSGPGSTGNR

SEQ ID NO: 804 - protein, 10 aa, Homo sapiens
QTVSWAVTPK

SEQ ID NO: 805 - protein, 7 aa, Homo sapiens
STFDALR

SEQ ID NO: 806 - protein, 21 aa, Homo sapiens
FEAAMQEKRNGDSHEDDEQSK

SEQ ID NO: 807 - protein, 9 aa, Homo sapiens
GGFGGGSFR

SEQ ID NO: 808 - protein, 11 aa, Homo sapiens
MEQTKNKFCVL

SEQ ID NO: 809 - protein, 13 aa, Homo sapiens
TLLVEAEGIEQEK

SEQ ID NO: 810 - protein, 17 aa, Homo sapiens
VEGTAFVIFGIQDGEQR

SEQ ID NO: 811 - protein, 14 aa, Homo sapiens
LWAYLTINQLLAER

SEQ ID NO: 812 - protein, 13 aa, Homo sapiens
LSNDMMGSYAEMK

SEQ ID NO: 813 - protein, 7 aa, Homo sapiens
NIFFSPL

SEQ ID NO: 814 - protein, 6 aa, Homo sapiens
QAALGV

SEQ ID NO: 815 - protein, 13 aa, Homo sapiens
GSPAINVAVHVFR

SEQ ID NO: 816 - protein, 12 aa, Homo sapiens
EAVMDINKPGPL

SEQ ID NO: 817 - protein, 8 aa, Homo sapiens
QSFDLSVK

SEQ ID NO: 818 - protein, 6 aa, Homo sapiens
SSAVMK

SEQ ID NO: 819 - protein, 14 aa, Homo sapiens
FHVPFTDLQVPSCK

SEQ ID NO: 820 - protein, 9 aa, Homo sapiens
GETGPQGQK

SEQ ID NO: 821 - protein, 10 aa, Homo sapiens
KRQQQAAQIR

SEQ ID NO: 822 - protein, 9 aa, Homo sapiens
QVVAGLNFR

SEQ ID NO: 823 - protein, 15 aa, Homo sapiens
IVNAGNLPVYDDIHK

SEQ ID NO: 824 - protein, 15 aa, Homo sapiens
QGQIIYNWQGAQSTQ

SEQ ID NO: 825 - protein, 7 aa, Homo sapiens
MLEEIMK

SEQ ID NO: 826 - protein, 6 aa, Homo sapiens
EASNLK

SEQ ID NO: 827 - protein, 7 aa, Homo sapiens
LAMQEFM

SEQ ID NO: 828 - protein, 11 aa, Homo sapiens
ENMQNAIVSIK

SEQ ID NO: 829 - protein, 14 aa, Homo sapiens
KHSVMMTFLSNMLR

SEQ ID NO: 830 - protein, 11 aa, Homo sapiens
LGEFVSETESR

SEQ ID NO: 831 - protein, 13 aa, Homo sapiens
WYLFGMGNEVDVH

SEQ ID NO: 832 - protein, 9 aa, Homo sapiens
NSEEFAAAM

SEQ ID NO: 833 - protein, 6 aa, Homo sapiens
IVTPQK

SEQ ID NO: 834 - protein, 22 aa, Homo sapiens
PTVSQQQSCPTCSTSLLNGHFK

SEQ ID NO: 835 - protein, 8 aa, Homo sapiens
FQVDNNNR

SEQ ID NO: 836 - protein, 10 aa, Homo sapiens
ISNIPDEYFK

SEQ ID NO: 837 - protein, 20 aa, Homo sapiens
EEFPFALGVQTLPQTCDEPK

SEQ ID NO: 838 - protein, 13 aa, Homo sapiens
AGSIHSKVSSYHG

SEQ ID NO: 839 - protein, 22 aa, Homo sapiens
GNDDHWIVDTDYDTYAVQYSCR

SEQ ID NO: 840 - protein, 19 aa, Homo sapiens
WQQGNIFSCSVMHEALHNR

SEQ ID NO: 841 - protein, 22 aa, Homo sapiens
NSTGKLVSAHALQTMFQLMTPK

SEQ ID NO: 842 - protein, 10 aa, Homo sapiens
AGILENGAVR

SEQ ID NO: 843 - protein, 7 aa, Homo sapiens
LYGSEAF

SEQ ID NO: 844 - protein, 10 aa, Homo sapiens
DNCPTVPNSA

SEQ ID NO: 845 - protein, 17 aa, Homo sapiens
QFCSTLTLNTAQANHTG

SEQ ID NO: 846 - protein, 10 aa, Homo sapiens
MGIIIPDFAR

SEQ ID NO: 847 - protein, 6 aa, Homo sapiens
QVTVPR

SEQ ID NO: 848 - protein, 10 aa, Homo sapiens
NQVSLTCLVK

SEQ ID NO: 849 - protein, 12 aa, Homo sapiens
TNYDEYAIFLTK

SEQ ID NO: 850 - protein, 9 aa, Homo sapiens
ALNLFQGSV

SEQ ID NO: 851 - protein, 7 aa, Homo sapiens
GDNAVIP

SEQ ID NO: 852 - protein, 7 aa, Homo sapiens
LPYSVVR

SEQ ID NO: 853 - protein, 7 aa, Homo sapiens
QADSVEQ

SEQ ID NO: 854 - protein, 30 aa, Homo sapiens
LNTDIAGLASAIDMSTNYNSDSLHFSNVFR

SEQ ID NO: 855 - protein, 10 aa, Homo sapiens
SVQEIQATFF

SEQ ID NO: 856 - protein, 15 aa, Homo sapiens
YILQGVTSWGLGCAR

SEQ ID NO: 857 - protein, 9 aa, Homo sapiens
DHAVDLIQK

SEQ ID NO: 858 - protein, 8 aa, Homo sapiens
HCLVTVEK

SEQ ID NO: 859 - protein, 7 aa, Homo sapiens
FLAENNK

SEQ ID NO: 860 - protein, 9 aa, Homo sapiens
FIAGAAPTK

SEQ ID NO: 861 - protein, 7 aa, Homo sapiens
LFLEPTR

SEQ ID NO: 862 - protein, 9 aa, Homo sapiens
IAGSGLVAK

SEQ ID NO: 863 - protein, 28 aa, Homo sapiens
VPFDAATLHTSTAMAAQHGMDDDGTGQK

SEQ ID NO: 864 - protein, 7 aa, Homo sapiens
MNLIKNK

SEQ ID NO: 865 - protein, 15 aa, Homo sapiens
TMTIHNGMFFSTYDR

SEQ ID NO: 866 - protein, 6 aa, Homo sapiens
DMVEYK

SEQ ID NO: 867 - protein, 8 aa, Homo sapiens
MNENSHVQ

SEQ ID NO: 868 - protein, 16 aa, Homo sapiens
EAEDLQVGQVELGGGP

SEQ ID NO: 869 - protein, 8 aa, Homo sapiens
DNSVHWER

SEQ ID NO: 870 - protein, 22 aa, Homo sapiens
VASPLDQNGSFNVVIKEEPLDD

SEQ ID NO: 871 - protein, 10 aa, Homo sapiens
SLGNVIMVCR

SEQ ID NO: 872 - protein, 15 aa, Homo sapiens
FSVPAGIVIPSFQAL

SEQ ID NO: 873 - protein, 15 aa, Homo sapiens
FCGQLGSPLGNPPGK

SEQ ID NO: 874 - protein, 15 aa, Homo sapiens
STPFDNEFYNGLCNR

SEQ ID NO: 875 - protein, 28 aa, Homo sapiens
CSPHLVLSALTSDNHGATYAFSGTHYWR

SEQ ID NO: 876 - protein, 21 aa, Homo sapiens
SYLSMVGSCCTSASPTVCFLK

SEQ ID NO: 877 - protein, 20 aa, Homo sapiens
LQELHLSSNGLESLSPEFLR

SEQ ID NO: 878 - protein, 6 aa, Homo sapiens
VPLVDR

SEQ ID NO: 879 - protein, 6 aa, Homo sapiens
SGNFNF

SEQ ID NO: 880 - protein, 14 aa, Homo sapiens
KLGNSGASPSSAGK

SEQ ID NO: 881 - protein, 20 aa, Homo sapiens
QTHQPPAPNSLIRFNAVLTN

SEQ ID NO: 882 - protein, 21 aa, Homo sapiens
MSAVEGICTSESPVIDHQGTK

SEQ ID NO: 883 - protein, 11 aa, Homo sapiens
CLPVTAPENGK

SEQ ID NO: 884 - protein, 29 aa, Homo sapiens
QDSLSSQNQLGVLPLSWDIPELVNMGQWK

SEQ ID NO: 885 - protein, 13 aa, Homo sapiens
QKINNYLTVPAHK

SEQ ID NO: 886 - protein, 7 aa, Homo sapiens
ANVPVLQ

SEQ ID NO: 887 - protein, 14 aa, Homo sapiens
ALNHLPLEYNSALY

SEQ ID NO: 888 - protein, 6 aa, Homo sapiens
INFNTR

SEQ ID NO: 889 - protein, 12 aa, Homo sapiens
AVPANGQTPIQR

SEQ ID NO: 890 - protein, 19 aa, Homo sapiens
LLNSIHENFSQAMASPAAR

SEQ ID NO: 891 - protein, 6 aa, Homo sapiens
LDALQR

SEQ ID NO: 892 - protein, 12 aa, Homo sapiens
VTFQLTYEEVLK

SEQ ID NO: 893 - protein, 10 aa, Homo sapiens
TTADGVARVR

SEQ ID NO: 894 - protein, 10 aa, Homo sapiens
LSLESLTSYF

SEQ ID NO: 895 - protein, 18 aa, Homo sapiens
KGGETSEMYLIQPDSSVK

SEQ ID NO: 896 - protein, 11 aa, Homo sapiens
LGADMEDVCGR

SEQ ID NO: 897 - protein, 15 aa, Homo sapiens
FSISGSYVLDQILPR

SEQ ID NO: 898 - protein, 10 aa, Homo sapiens
QFTSSTSYNR

SEQ ID NO: 899 - protein, 14 aa, Homo sapiens
SPVGVQPILNEHTF

SEQ ID NO: 900 - protein, 18 aa, Homo sapiens
VFSNGADLSGVTEEAPLK

SEQ ID NO: 901 - protein, 12 aa, Homo sapiens
RPASPISTIQPK

SEQ ID NO: 902 - protein, 7 aa, Homo sapiens
LSCMAIR

SEQ ID NO: 903 - protein, 14 aa, Homo sapiens
AGDFLEANYMNLQR

SEQ ID NO: 904 - protein, 8 aa, Homo sapiens
QLEWGLER

SEQ ID NO: 905 - protein, 18 aa, Homo sapiens
SPVGVQPILNEHTFCAGM

SEQ ID NO: 906 - protein, 8 aa, Homo sapiens
ETLLQDFR

SEQ ID NO: 907 - protein, 19 aa, Homo sapiens
VVAQGVGIPEDSIFTMADR

SEQ ID NO: 908 - protein, 13 aa, Homo sapiens
FATTFYQHLADSK

SEQ ID NO: 909 - protein, 8 aa, Homo sapiens
NSELMDPK

SEQ ID NO: 910 - protein, 12 aa, Homo sapiens
GPWCFTTDPSVR

SEQ ID NO: 911 - protein, 11 aa, Homo sapiens
GEGPATIKTFR

SEQ ID NO: 912 - protein, 12 aa, Homo sapiens
GYDNGIIWATWK

SEQ ID NO: 913 - protein, 14 aa, Homo sapiens
AFDGFDFGDDPSDK

SEQ ID NO: 914 - protein, 8 aa, Homo sapiens
NQSLKSVK

SEQ ID NO: 915 - protein, 19 aa, Homo sapiens
VQLSPDLLATLPEPASPGR

SEQ ID NO: 916 - protein, 19 aa, Homo sapiens
VACEEPPFIENGAANLHSK

SEQ ID NO: 917 - protein, 6 aa, Homo sapiens
QASAIK

SEQ ID NO: 918 - protein, 20 aa, Homo sapiens
VPSYTLILPSLELPVLHVPR

SEQ ID NO: 919 - protein, 10 aa, Homo sapiens
SSGSSCNSTR

SEQ ID NO: 920 - protein, 24 aa, Homo sapiens
ADSQAQLLLSTVVGVFTAPGLHLK

SEQ ID NO: 921 - protein, 6 aa, Homo sapiens
SILKDF

SEQ ID NO: 922 - protein, 8 aa, Homo sapiens
RGVTTKSF

SEQ ID NO: 923 - protein, 8 aa, Homo sapiens
LTLDIQNK

SEQ ID NO: 924 - protein, 18 aa, Homo sapiens
TKEEYGHSEVVEYYCNPR

SEQ ID NO: 925 - protein, 27 aa, Homo sapiens
RPCGHPGDTPFGTFTLTGGNVFEYGVK

SEQ ID NO: 926 - protein, 15 aa, Homo sapiens
SSSNEEVMFLTVQVK

SEQ ID NO: 927 - protein, 17 aa, Homo sapiens
TCPKPDDLPFSTVVPLK

SEQ ID NO: 928 - protein, 19 aa, Homo sapiens
ATTVTGTPCQDWAAQEPHR

SEQ ID NO: 929 - protein, 23 aa, Homo sapiens
VVGLIQDLKPNTIMVLVNYIHFK

SEQ ID NO: 930 - protein, 13 aa, Homo sapiens
DGVVSLWSSATGK

SEQ ID NO: 931 - protein, 13 aa, Homo sapiens
KTLMAAGDKQRPR

SEQ ID NO: 932 - protein, 6 aa, Homo sapiens
DTNDEK

SEQ ID NO: 933 - protein, 24 aa, Homo sapiens
TMSGLECQAWDSQSPHAHGYIPSK

SEQ ID NO: 934 - protein, 12 aa, Homo sapiens
VTAAPQSVCALR

SEQ ID NO: 935 - protein, 15 aa, Homo sapiens
DKICDLLVANNHFAH

SEQ ID NO: 936 - protein, 10 aa, Homo sapiens
VDKDLQSLED

SEQ ID NO: 937 - protein, 8 aa, Homo sapiens
DGFFGNPR

SEQ ID NO: 938 - protein, 15 aa, Homo sapiens
FYGDEISFSCHETSR

SEQ ID NO: 939 - protein, 15 aa, Homo sapiens
LSPTGTTEFWLGNEK

SEQ ID NO: 940 - protein, 8 aa, Homo sapiens
ILDDLSPR

SEQ ID NO: 941 - protein, 9 aa, Homo sapiens
DIAPTLTLY

SEQ ID NO: 942 - protein, 18 aa, Homo sapiens
HPNSPLDEENLTQENQDR

SEQ ID NO: 943 - protein, 12 aa, Homo sapiens
DTAVFECLPQHA

SEQ ID NO: 944 - protein, 13 aa, Homo sapiens
PSLVPASAENVNK

SEQ ID NO: 945 - protein, 8 aa, Homo sapiens
QWAGLVEK

SEQ ID NO: 946 - protein, 7 aa, Homo sapiens
AQLVDMK

SEQ ID NO: 947 - protein, 28 aa, Homo sapiens
GMADQDGLKPTIDKPSEDSPPLEMLGPR

SEQ ID NO: 948 - protein, 19 aa, Homo sapiens
FMPAAQLPELPDVELPTNK

SEQ ID NO: 949 - protein, 14 aa, Homo sapiens
EGFGHLSPTGTTEF

SEQ ID NO: 950 - protein, 10 aa, Homo sapiens
GGVEDEVTLS

SEQ ID NO: 951 - protein, 16 aa, Homo sapiens
VGSCCTSASPTVCFLK

SEQ ID NO: 952 - protein, 12 aa, Homo sapiens
TLMFGSYLDDEK

SEQ ID NO: 953 - protein, 15 aa, Homo sapiens
IQNAHLEDAGNYNCR

SEQ ID NO: 954 - protein, 25 aa, Homo sapiens
RFLKGQDEDQVHSVPIAQMGNYQEY

SEQ ID NO: 955 - protein, 13 aa, Homo sapiens
SSLSVPYVIVPLK

SEQ ID NO: 956 - protein, 7 aa, Homo sapiens
PLGEEMR

SEQ ID NO: 957 - protein, 24 aa, Homo sapiens
VGGTLGQFYQEVLWGSPAASDDGR

SEQ ID NO: 958 - protein, 12 aa, Homo sapiens
KDGVGMVEYLRK

SEQ ID NO: 959 - protein, 8 aa, Homo sapiens
QQNAQGGF

SEQ ID NO: 960 - protein, 10 aa, Homo sapiens
QAQQMVQPQS

SEQ ID NO: 961 - protein, 10 aa, Homo sapiens
VLTPDAFVCR

SEQ ID NO: 962 - protein, 6 aa, Homo sapiens
QSGLYF

SEQ ID NO: 963 - protein, 9 aa, Homo sapiens
VDGALCMEK

SEQ ID NO: 964 - protein, 7 aa, Homo sapiens
QIGSVYR

SEQ ID NO: 965 - protein, 8 aa, Homo sapiens
KNFATSNK

SEQ ID NO: 966 - protein, 12 aa, Homo sapiens
SPVGVQPILNEH

SEQ ID NO: 967 - protein, 7 aa, Homo sapiens
AALLNAI

SEQ ID NO: 968 - protein, 11 aa, Homo sapiens
DGGDDGNLVIK

SEQ ID NO: 969 - protein, 11 aa, Homo sapiens
NPMACNGSAAR

SEQ ID NO: 970 - protein, 9 aa, Homo sapiens
VGYVSGWGR

SEQ ID NO: 971 - protein, 22 aa, Homo sapiens
ETTFNSLLCPSGGEVSEELSLK

SEQ ID NO: 972 - protein, 15 aa, Homo sapiens
EIGILTTNKNTSRTH

SEQ ID NO: 973 - protein, 15 aa, Homo sapiens
EWVAIESDSVQPVPR

SEQ ID NO: 974 - protein, 16 aa, Homo sapiens
QAMQEDRAPSSGNSTR

SEQ ID NO: 975 - protein, 8 aa, Homo sapiens
SLHMYANR

SEQ ID NO: 976 - protein, 12 aa, Homo sapiens
NPNGYSFSIPVK

SEQ ID NO: 977 - protein, 6 aa, Homo sapiens
QGQELK

SEQ ID NO: 978 - protein, 8 aa, Homo sapiens
TPENFPCK

SEQ ID NO: 979 - protein, 8 aa, Homo sapiens
SSSAWERK

SEQ ID NO: 980 - protein, 7 aa, Homo sapiens
VGAAAVL

SEQ ID NO: 981 - protein, 7 aa, Homo sapiens
ILEICTR

SEQ ID NO: 982 - protein, 18 aa, Homo sapiens
TSDNHGATYAFSGTHYWR

SEQ ID NO: 983 - protein, 11 aa, Homo sapiens
IETILLNGTDR

SEQ ID NO: 984 - protein, 18 aa, Homo sapiens
TTLSGAPCQPWASEATYR

SEQ ID NO: 985 - protein, 17 aa, Homo sapiens
KPVAFSDYIHPVCLPDR

SEQ ID NO: 986 - protein, 21 aa, Homo sapiens
EQNQTSANNMRHLTAENNQER

SEQ ID NO: 987 - protein, 8 aa, Homo sapiens
VNLNEEPT

SEQ ID NO: 988 - protein, 7 aa, Homo sapiens
FDTISEK

SEQ ID NO: 989 - protein, 11 aa, Homo sapiens
ELEASKVVLLP

SEQ ID NO: 990 - protein, 9 aa, Homo sapiens
EDEAVLVLG

SEQ ID NO: 991 - protein, 14 aa, Homo sapiens
LELELRPTGEIEQY

SEQ ID NO: 992 - protein, 6 aa, Homo sapiens
LCVDPR

SEQ ID NO: 993 - protein, 15 aa, Homo sapiens
QDACQGDSGGVFAVR

SEQ ID NO: 994 - protein, 20 aa, Homo sapiens
YTYNYEAESSSGVPGTADSR

SEQ ID NO: 995 - protein, 22 aa, Homo sapiens
VPAPPSPQPATYTCVVSHEDSR

SEQ ID NO: 996 - protein, 11 aa, Homo sapiens
SNNYLHLSVLR

SEQ ID NO: 997 - protein, 20 aa, Homo sapiens
LEIQSQVDSQHVGHSVLTAK

SEQ ID NO: 998 - protein, 17 aa, Homo sapiens
LFAVYDQSATALHFLGR

SEQ ID NO: 999 - protein, 12 aa, Homo sapiens
DTEDQEDQVDPR

SEQ ID NO: 1000 - protein, 10 aa, Homo sapiens
LHENNWICIQ

SEQ ID NO: 1001 - protein, 11 aa, Homo sapiens
LKLMKEAVLVK

SEQ ID NO: 1002 - protein, 11 aa, Homo sapiens
HEGSFIQGAEK

SEQ ID NO: 1003 - protein, 8 aa, Homo sapiens
LCTPLLPK

SEQ ID NO: 1004 - protein, 10 aa, Homo sapiens
DSCVGSLVVK

SEQ ID NO: 1005 - protein, 20 aa, Homo sapiens
QLVHHFEIDVDIFEPQGISK

SEQ ID NO: 1006 - protein, 9 aa, Homo sapiens
LNPPDTDIR

SEQ ID NO: 1007 - protein, 16 aa, Homo sapiens
GEAFTLKATVLNYLPK

SEQ ID NO: 1008 - protein, 20 aa, Homo sapiens
SPVGVQPILNEHTFCAGMSK

SEQ ID NO: 1009 - protein, 8 aa, Homo sapiens
LNGESNLR

SEQ ID NO: 1010 - protein, 23 aa, Homo sapiens
QKPDGVFQEDAPVIHQEMIGGLR

SEQ ID NO: 1011 - protein, 20 aa, Homo sapiens
DKDQEVLLQTFLDDASPGDK

SEQ ID NO: 1012 - protein, 13 aa, Homo sapiens
AFVIFGIQDGEQR

SEQ ID NO: 1013 - protein, 9 aa, Homo sapiens
EQCSNMQPK

SEQ ID NO: 1014 - protein, 16 aa, Homo sapiens
STDTSCVNPPTVQNAY

SEQ ID NO: 1015 - protein, 10 aa, Homo sapiens
MVLVNYIFFK

SEQ ID NO: 1016 - protein, 7 aa, Homo sapiens
SSVITLN

SEQ ID NO: 1017 - protein, 13 aa, Homo sapiens
GDYTLNNVHACAK

SEQ ID NO: 1018 - protein, 22 aa, Homo sapiens
GLLGGGVSVEDCCLNTAFAYQK

SEQ ID NO: 1019 - protein, 28 aa, Homo sapiens
SPCYGYQWVSEEHEEAHHTAYLVFSPSK

SEQ ID NO: 1020 - protein, 13 aa, Homo sapiens
IQEEGTVVELTGR

SEQ ID NO: 1021 - protein, 12 aa, Homo sapiens
TEGDGVYTLNDK

SEQ ID NO: 1022 - protein, 9 aa, Homo sapiens
MLEVPYVDR

SEQ ID NO: 1023 - protein, 8 aa, Homo sapiens
YTVILDNA

SEQ ID NO: 1024 - protein, 7 aa, Homo sapiens
TMQALPY

SEQ ID NO: 1025 - protein, 12 aa, Homo sapiens
NRQAAAANPENS

SEQ ID NO: 1026 - protein, 9 aa, Homo sapiens
AGAEPASER

SEQ ID NO: 1027 - protein, 14 aa, Homo sapiens
LSGSSRGGGPLPLD

SEQ ID NO: 1028 - protein, 17 aa, Homo sapiens
VYACEVTHQGLSSPVTK

SEQ ID NO: 1029 - protein, 16 aa, Homo sapiens
VHQYFNVELIQPGAVK

SEQ ID NO: 1030 - protein, 29 aa, Homo sapiens
QPNCDDPETEEAALVAIDYINQNLPWGYK

SEQ ID NO: 1031 - protein, 7 aa, Homo sapiens
IEIPLPF

SEQ ID NO: 1032 - protein, 11 aa, Homo sapiens
AREDIFMETLK

SEQ ID NO: 1033 - protein, 13 aa, Homo sapiens
QGFGNVATNTDGK

SEQ ID NO: 1034 - protein, 19 aa, Homo sapiens
KGEVLPLPEANFPSFPLPH

SEQ ID NO: 1035 - protein, 22 aa, Homo sapiens
VGINGEANLDFLNIPLTIPEMR

SEQ ID NO: 1036 - protein, 6 aa, Homo sapiens
LAHQQQ

SEQ ID NO: 1037 - protein, 16 aa, Homo sapiens
SCDNPYIPNGDYSPLR

SEQ ID NO: 1038 - protein, 15 aa, Homo sapiens
DLGTLSGIGTLDGFR

SEQ ID NO: 1039 - protein, 8 aa, Homo sapiens
DYFIATCK

SEQ ID NO: 1040 - protein, 6 aa, Homo sapiens
KEDGLR

SEQ ID NO: 1041 - protein, 7 aa, Homo sapiens
EVEELAR

SEQ ID NO: 1042 - protein, 10 aa, Homo sapiens
DGAEVMSPLK

SEQ ID NO: 1043 - protein, 13 aa, Homo sapiens
HGNTDSEGIVEVK

SEQ ID NO: 1044 - protein, 25 aa, Homo sapiens
EGDSYHNATTVNGTPVNHQPLERHR

SEQ ID NO: 1045 - protein, 18 aa, Homo sapiens
YSDASDCHGEDSQAFCEK

SEQ ID NO: 1046 - protein, 19 aa, Homo sapiens
LGEHNIDVLEGNEQFINAA

SEQ ID NO: 1047 - protein, 6 aa, Homo sapiens
SLFLSK

SEQ ID NO: 1048 - protein, 24 aa, Homo sapiens
YFKPGMPFDLMVFVTNPDGSPAYR

SEQ ID NO: 1049 - protein, 7 aa, Homo sapiens
GPGEDFR

SEQ ID NO: 1050 - protein, 13 aa, Homo sapiens
FQSQADQDQQASG

SEQ ID NO: 1051 - protein, 9 aa, Homo sapiens
GSEMVVAGK

SEQ ID NO: 1052 - protein, 25 aa, Homo sapiens
TGCSLTGASVDSTLAFNTYVHFQGK

SEQ ID NO: 1053 - protein, 10 aa, Homo sapiens
VFKNIKTVMK

SEQ ID NO: 1054 - protein, 11 aa, Homo sapiens
EDVYVVGTVLR

SEQ ID NO: 1055 - protein, 11 aa, Homo sapiens
LICQATGFSPR

SEQ ID NO: 1056 - protein, 10 aa, Homo sapiens
IISDYHQQFR

SEQ ID NO: 1057 - protein, 15 aa, Homo sapiens
DCHLAQVPSHTVVAR

SEQ ID NO: 1058 - protein, 6 aa, Homo sapiens
MGFGVK

SEQ ID NO: 1059 - protein, 23 aa, Homo sapiens
EPTMYGEILSPNYPQAYPSEVEK

SEQ ID NO: 1060 - protein, 12 aa, Homo sapiens
LDTLAQEVALLK

SEQ ID NO: 1061 - protein, 15 aa, Homo sapiens
VDLSFSPSQSLPASH

SEQ ID NO: 1062 - protein, 18 aa, Homo sapiens
QTLATPAMGDIQIGMEDK

SEQ ID NO: 1063 - protein, 18 aa, Homo sapiens
VGLSGMAIADVTLLSGFH

SEQ ID NO: 1064 - protein, 8 aa, Homo sapiens
SFVHLEPM

SEQ ID NO: 1065 - protein, 17 aa, Homo sapiens
IITHPNFNGNTLDNDIM

SEQ ID NO: 1066 - protein, 16 aa, Homo sapiens
VELLHNPAFCSLATTK

SEQ ID NO: 1067 - protein, 9 aa, Homo sapiens
EGLEYIPLR

SEQ ID NO: 1068 - protein, 9 aa, Homo sapiens
CTEGFNVDK

SEQ ID NO: 1069 - protein, 15 aa, Homo sapiens
FDHVITNMNNNYEPR

SEQ ID NO: 1070 - protein, 8 aa, Homo sapiens
IQQTIAAN

SEQ ID NO: 1071 - protein, 14 aa, Homo sapiens
LNTDIAGLASAIDM

SEQ ID NO: 1072 - protein, 8 aa, Homo sapiens
QIDDIDVR

SEQ ID NO: 1073 - protein, 14 aa, Homo sapiens
CLDPCVISQEIMEK

SEQ ID NO: 1074 - protein, 8 aa, Homo sapiens
LKEIQMSE

SEQ ID NO: 1075 - protein, 6 aa, Homo sapiens
SPSSPL

SEQ ID NO: 1076 - protein, 6 aa, Homo sapiens
QAPLLR

SEQ ID NO: 1077 - protein, 8 aa, Homo sapiens
KLVLSSEK

SEQ ID NO: 1078 - protein, 6 aa, Homo sapiens
HTQGGK

SEQ ID NO: 1079 - protein, 6 aa, Homo sapiens
RIGGIK

SEQ ID NO: 1080 - protein, 8 aa, Homo sapiens
NYVDWIMK

SEQ ID NO: 1081 - protein, 9 aa, Homo sapiens
GQSTGDTTI

SEQ ID NO: 1082 - protein, 8 aa, Homo sapiens
EHVAHLLF

SEQ ID NO: 1083 - protein, 9 aa, Homo sapiens
GMALFGEGK

SEQ ID NO: 1084 - protein, 7 aa, Homo sapiens
NDFFLHY

SEQ ID NO: 1085 - protein, 6 aa, Homo sapiens
LQAEAF

SEQ ID NO: 1086 - protein, 8 aa, Homo sapiens
SSEDIRCK

SEQ ID NO: 1087 - protein, 19 aa, Homo sapiens
TFQIPGYTVPVVNVEVSPF

SEQ ID NO: 1088 - protein, 7 aa, Homo sapiens
QLQASLS

SEQ ID NO: 1089 - protein, 19 aa, Homo sapiens
MRPSTDTITVMVENSHGLR

SEQ ID NO: 1090 - protein, 8 aa, Homo sapiens
DLRSGTLY

SEQ ID NO: 1091 - protein, 6 aa, Homo sapiens
DTIVFK

SEQ ID NO: 1092 - protein, 11 aa, Homo sapiens
ADLIPYQDSPE

SEQ ID NO: 1093 - protein, 7 aa, Homo sapiens
QAQDSSL

SEQ ID NO: 1094 - protein, 15 aa, Homo sapiens
VVHTNYDEYAIFLTK

SEQ ID NO: 1095 - protein, 8 aa, Homo sapiens
VDDGVASF

SEQ ID NO: 1096 - protein, 10 aa, Homo sapiens
GAEVNALDGY

SEQ ID NO: 1097 - protein, 11 aa, Homo sapiens
VAEYMDWILEK

SEQ ID NO: 1098 - protein, 6 aa, Homo sapiens
LDSAPF

SEQ ID NO: 1099 - protein, 9 aa, Homo sapiens
LISLLNEKG

SEQ ID NO: 1100 - protein, 18 aa, Homo sapiens
VFAPTLLTVAVHFEEVAK

SEQ ID NO: 1101 - protein, 18 aa, Homo sapiens
ALFLSPSAQQASWQVSAR

SEQ ID NO: 1102 - protein, 17 aa, Homo sapiens
VTLTCVAPLSGVDFQLR

SEQ ID NO: 1103 - protein, 16 aa, Homo sapiens
KQCSSLQTAIADAEQR

SEQ ID NO: 1104 - protein, 13 aa, Homo sapiens
EPANGAQNPGPAK

SEQ ID NO: 1105 - protein, 6 aa, Homo sapiens
LIFPSA

SEQ ID NO: 1106 - protein, 8 aa, Homo sapiens
NSLPPSHQ

SEQ ID NO: 1107 - protein, 15 aa, Homo sapiens
ISRLQAEIEGLKGQR

SEQ ID NO: 1108 - protein, 20 aa, Homo sapiens
DTWVEHWPEEDECQDEENQK

SEQ ID NO: 1109 - protein, 14 aa, Homo sapiens
SGIPIVTSPYQIHF

SEQ ID NO: 1110 - protein, 12 aa, Homo sapiens
QDTYLQIAAFTR

SEQ ID NO: 1111 - protein, 12 aa, Homo sapiens
FLVGPDGIPIMR

SEQ ID NO: 1112 - protein, 15 aa, Homo sapiens
LYLVQGTQVYVFLTK

SEQ ID NO: 1113 - protein, 8 aa, Homo sapiens
FEDCCQEK

SEQ ID NO: 1114 - protein, 12 aa, Homo sapiens
AATGECTATVGK

SEQ ID NO: 1115 - protein, 9 aa, Homo sapiens
VATENTDEQ

SEQ ID NO: 1116 - protein, 9 aa, Homo sapiens
DIQVIVNVP

SEQ ID NO: 1117 - protein, 7 aa, Homo sapiens
EQMGEAM

SEQ ID NO: 1118 - protein, 18 aa, Homo sapiens
NHLQLEGLFFTNGEHTSK

SEQ ID NO: 1119 - protein, 10 aa, Homo sapiens
MLAFDVNDEK

SEQ ID NO: 1120 - protein, 16 aa, Homo sapiens
EPMKVQDSVSIKADNT

SEQ ID NO: 1121 - protein, 12 aa, Homo sapiens
EVQLVQSGAEVK

SEQ ID NO: 1122 - protein, 17 aa, Homo sapiens
SVLEGGVIPLQDLSGLK

SEQ ID NO: 1123 - protein, 7 aa, Homo sapiens
CVEISCK

SEQ ID NO: 1124 - protein, 6 aa, Homo sapiens
ANDNIR

SEQ ID NO: 1125 - protein, 9 aa, Homo sapiens
FISLGEACK

SEQ ID NO: 1126 - protein, 8 aa, Homo sapiens
NLDENYCR

SEQ ID NO: 1127 - protein, 13 aa, Homo sapiens
YYWGGQYTWDMAK

SEQ ID NO: 1128 - protein, 21 aa, Homo sapiens
TECFITGWGETQGTFGAGLLK

SEQ ID NO: 1129 - protein, 7 aa, Homo sapiens
SILENLR

SEQ ID NO: 1130 - protein, 9 aa, Homo sapiens
TSCLLFMGR

SEQ ID NO: 1131 - protein, 14 aa, Homo sapiens
SMNIDGMTYPGIIK

SEQ ID NO: 1132 - protein, 12 aa, Homo sapiens
ISHTIEVPTFGK

SEQ ID NO: 1133 - protein, 8 aa, Homo sapiens
MIDAATLK

SEQ ID NO: 1134 - protein, 8 aa, Homo sapiens
ISCTIANR

SEQ ID NO: 1135 - protein, 8 aa, Homo sapiens
SLLVSYTK

SEQ ID NO: 1136 - protein, 7 aa, Homo sapiens
WILTAAH

SEQ ID NO: 1137 - protein, 6 aa, Homo sapiens
DSEGRR

SEQ ID NO: 1138 - protein, 24 aa, Homo sapiens
FDLVPVPTNLYGDFFTGDAYVILK

SEQ ID NO: 1139 - protein, 8 aa, Homo sapiens
SILGSDVR

SEQ ID NO: 1140 - protein, 12 aa, Homo sapiens
HYYIAAEEIIWN

SEQ ID NO: 1141 - protein, 6 aa, Homo sapiens
QGCTGK

SEQ ID NO: 1142 - protein, 7 aa, Homo sapiens
LVLSSEK

SEQ ID NO: 1143 - protein, 25 aa, Homo sapiens
QHQTVLELTETGVEAAAASAISVAR

SEQ ID NO: 1144 - protein, 8 aa, Homo sapiens
QAKNVDAI

SEQ ID NO: 1145 - protein, 7 aa, Homo sapiens
LLQALVR

SEQ ID NO: 1146 - protein, 22 aa, Homo sapiens
VILGAHQEVNLEPHVQEIEVSR

SEQ ID NO: 1147 - protein, 11 aa, Homo sapiens
SASNMAIVDVK

SEQ ID NO: 1148 - protein, 10 aa, Homo sapiens
LEKGQQQPLE

SEQ ID NO: 1149 - protein, 19 aa, Homo sapiens
VSNQTLSLFFTVLQDVPVR

SEQ ID NO: 1150 - protein, 25 aa, Homo sapiens
LEGAIHLQEQDMENSRQVLNSYEVL

SEQ ID NO: 1151 - protein, 7 aa, Homo sapiens
SNSAMVK

SEQ ID NO: 1152 - protein, 19 aa, Homo sapiens
DLKPQNLLINTEGAIKLAD

SEQ ID NO: 1153 - protein, 13 aa, Homo sapiens
DMPASEDLQDLQK

SEQ ID NO: 1154 - protein, 8 aa, Homo sapiens
ASWQVSAR

SEQ ID NO: 1155 - protein, 9 aa, Homo sapiens
VGGQKEGAR

SEQ ID NO: 1156 - protein, 8 aa, Homo sapiens
MCPQLQQY

SEQ ID NO: 1157 - protein, 8 aa, Homo sapiens
NGMLHGDK

SEQ ID NO: 1158 - protein, 11 aa, Homo sapiens
NPVLAPPSLSK

SEQ ID NO: 1159 - protein, 29 aa, Homo sapiens
TDELPQLVTLPHPNLHGPEILDVPSTVQK

SEQ ID NO: 1160 - protein, 6 aa, Homo sapiens
EVAGIK

SEQ ID NO: 1161 - protein, 11 aa, Homo sapiens
DTQRPSGIPQR

SEQ ID NO: 1162 - protein, 28 aa, Homo sapiens
NLVINCEVFDPQEHENINGVPPHLGHPF

SEQ ID NO: 1163 - protein, 14 aa, Homo sapiens
QKVENLFNEKCGEA

SEQ ID NO: 1164 - protein, 7 aa, Homo sapiens
TFLPLNK

SEQ ID NO: 1165 - protein, 25 aa, Homo sapiens
YHFEALADTGISSEFYDNANDLLSK

SEQ ID NO: 1166 - protein, 7 aa, Homo sapiens
EHSGGGK

SEQ ID NO: 1167 - protein, 6 aa, Homo sapiens
DIGYSF

SEQ ID NO: 1168 - protein, 7 aa, Homo sapiens
LDKGQLR

SEQ ID NO: 1169 - protein, 10 aa, Homo sapiens
IEIPLPFGGK

SEQ ID NO: 1170 - protein, 19 aa, Homo sapiens
HNVYINGITYTPVSSTNEK

SEQ ID NO: 1171 - protein, 16 aa, Homo sapiens
TLILPSLELPVLHVPR

SEQ ID NO: 1172 - protein, 15 aa, Homo sapiens
QDGETGGHRRSFPPR

SEQ ID NO: 1173 - protein, 11 aa, Homo sapiens
AEDVGYQSSSR

SEQ ID NO: 1174 - protein, 12 aa, Homo sapiens
YNILPEKEEFPF

SEQ ID NO: 1175 - protein, 9 aa, Homo sapiens
VPQTDMTFR

SEQ ID NO: 1176 - protein, 21 aa, Homo sapiens
LNPSQNATGTSRSESPVCSSR

SEQ ID NO: 1177 - protein, 18 aa, Homo sapiens
FSEGTAGDSLSLHSGRPF

SEQ ID NO: 1178 - protein, 19 aa, Homo sapiens
MTTDVASSRLQRLDNSTVR

SEQ ID NO: 1179 - protein, 9 aa, Homo sapiens
TGLQEVEVK

SEQ ID NO: 1180 - protein, 13 aa, Homo sapiens
SASSGVPNPPKAR

SEQ ID NO: 1181 - protein, 19 aa, Homo sapiens
EVSSCHCAPCQGNGVPVLK

SEQ ID NO: 1182 - protein, 13 aa, Homo sapiens
AFTECCVVASQLR

SEQ ID NO: 1183 - protein, 11 aa, Homo sapiens
SEYQADYESLR

SEQ ID NO: 1184 - protein, 7 aa, Homo sapiens
ELRPWCF

SEQ ID NO: 1185 - protein, 15 aa, Homo sapiens
DIPTNSPELEETLTH

SEQ ID NO: 1186 - protein, 6 aa, Homo sapiens
VQFELH

SEQ ID NO: 1187 - protein, 12 aa, Homo sapiens
MGQPGNGSAFLL

SEQ ID NO: 1188 - protein, 16 aa, Homo sapiens
QFSFPLSSEPFQGSYK

SEQ ID NO: 1189 - protein, 21 aa, Homo sapiens
NPFVFAPTLLTVAVHFEEVAK

SEQ ID NO: 1190 - protein, 16 aa, Homo sapiens
NMEQFQVSVSVAPNAK

SEQ ID NO: 1191 - protein, 13 aa, Homo sapiens
DKICDLLVANNHF

SEQ ID NO: 1192 - protein, 11 aa, Homo sapiens
LIENGYFHPVK

SEQ ID NO: 1193 - protein, 12 aa, Homo sapiens
EGWIHTVCINGR

SEQ ID NO: 1194 - protein, 8 aa, Homo sapiens
ASSFLGEK

SEQ ID NO: 1195 - protein, 18 aa, Homo sapiens
FNAVLTNPQGDYDTSTGK

SEQ ID NO: 1196 - protein, 18 aa, Homo sapiens
ECDTDGWTNDIPICEVVK

SEQ ID NO: 1197 - protein, 12 aa, Homo sapiens
VTFELTYEELLK

SEQ ID NO: 1198 - protein, 10 aa, Homo sapiens
EDLIWELLNQ

SEQ ID NO: 1199 - protein, 10 aa, Homo sapiens
FEDDIDNCPF

SEQ ID NO: 1200 - protein, 21 aa, Homo sapiens
TPEVTCVVVDVSHEDPEVQFK

SEQ ID NO: 1201 - protein, 6 aa, Homo sapiens
QKHQGR

SEQ ID NO: 1202 - protein, 12 aa, Homo sapiens
ALQDQLVLVAAK

SEQ ID NO: 1203 - protein, 23 aa, Homo sapiens
YVSHFETEGPHVLLYFDSVPTSR

SEQ ID NO: 1204 - protein, 17 aa, Homo sapiens
AQTTVTCMENGWSPTPR

SEQ ID NO: 1205 - protein, 19 aa, Homo sapiens
FDHTNSLNIAGLSLDFSSK

SEQ ID NO: 1206 - protein, 11 aa, Homo sapiens
AGFSWIEVTFK

SEQ ID NO: 1207 - protein, 13 aa, Homo sapiens
GLEEELQFSLGSK

SEQ ID NO: 1208 - protein, 16 aa, Homo sapiens
VEKPTADAEAYVFTPN

SEQ ID NO: 1209 - protein, 8 aa, Homo sapiens
TTNIQGIN

SEQ ID NO: 1210 - protein, 8 aa, Homo sapiens
REFLQELR

SEQ ID NO: 1211 - protein, 9 aa, Homo sapiens
QQQAAPNLR

SEQ ID NO: 1212 - protein, 9 aa, Homo sapiens
LLGLSLAGK

SEQ ID NO: 1213 - protein, 12 aa, Homo sapiens
TWQDCEYKDAAK

SEQ ID NO: 1214 - protein, 20 aa, Homo sapiens
RHDEFERHAEGTFTSDVSSY

SEQ ID NO: 1215 - protein, 12 aa, Homo sapiens
AFESSFWHMQEK

SEQ ID NO: 1216 - protein, 10 aa, Homo sapiens
VGPGPALMLR

SEQ ID NO: 1217 - protein, 18 aa, Homo sapiens
CSYTEDAQCIDGTIEVPK

SEQ ID NO: 1218 - protein, 16 aa, Homo sapiens
VAGLLEDTFPGLLGLR

SEQ ID NO: 1219 - protein, 6 aa, Homo sapiens
NFGVSL

SEQ ID NO: 1220 - protein, 8 aa, Homo sapiens
FQQDVMAR

SEQ ID NO: 1221 - protein, 11 aa, Homo sapiens
YGLVTYATYPK

SEQ ID NO: 1222 - protein, 14 aa, Homo sapiens
DTVIKPLLVEPEGL

SEQ ID NO: 1223 - protein, 13 aa, Homo sapiens
TSLVIENEAGDER

SEQ ID NO: 1224 - protein, 27 aa, Homo sapiens
SVLRPSQFGGQPCTAPLVAFQPCIPSK

SEQ ID NO: 1225 - protein, 7 aa, Homo sapiens
EDPLLEK

SEQ ID NO: 1226 - protein, 7 aa, Homo sapiens
LAIPEGK

SEQ ID NO: 1227 - protein, 19 aa, Homo sapiens
KLYEQHHVVQDMLVPMKCL

SEQ ID NO: 1228 - protein, 11 aa, Homo sapiens
ENVVTTFAPNR

SEQ ID NO: 1229 - protein, 11 aa, Homo sapiens
VEDTAVYYCAR

SEQ ID NO: 1230 - protein, 10 aa, Homo sapiens
QMNKNHGILR

SEQ ID NO: 1231 - protein, 13 aa, Homo sapiens
SVSLPSLDPASAK

SEQ ID NO: 1232 - protein, 9 aa, Homo sapiens
GTYGLSCQR

SEQ ID NO: 1233 - protein, 19 aa, Homo sapiens
ASVSVLGDILGSAMQNTQN

SEQ ID NO: 1234 - protein, 11 aa, Homo sapiens
VDTGHLGQVER

SEQ ID NO: 1235 - protein, 12 aa, Homo sapiens
WYVDGVQVHNAK

SEQ ID NO: 1236 - protein, 12 aa, Homo sapiens
QAVTVKEALSTK

SEQ ID NO: 1237 - protein, 6 aa, Homo sapiens
NSSYGK

SEQ ID NO: 1238 - protein, 23 aa, Homo sapiens
NTEILTGSWSDQTYPEGTQAIYK

SEQ ID NO: 1239 - protein, 14 aa, Homo sapiens
FRDEELSCTVVELK

SEQ ID NO: 1240 - protein, 6 aa, Homo sapiens
MPVSQK

SEQ ID NO: 1241 - protein, 28 aa, Homo sapiens
FSSHVGGTLGQFYQEVLWGSPAASDDGR

SEQ ID NO: 1242 - protein, 9 aa, Homo sapiens
SGGLCQPCR

SEQ ID NO: 1243 - protein, 14 aa, Homo sapiens
GYSYHDGYGEALGR

SEQ ID NO: 1244 - protein, 19 aa, Homo sapiens
SIFQDDFVIPDISEPGTWK

SEQ ID NO: 1245 - protein, 14 aa, Homo sapiens
IREEGTDLEVTANR

SEQ ID NO: 1246 - protein, 6 aa, Homo sapiens
DKSVVC

SEQ ID NO: 1247 - protein, 6 aa, Homo sapiens
YNINGK

SEQ ID NO: 1248 - protein, 6 aa, Homo sapiens
YLELYR

SEQ ID NO: 1249 - protein, 18 aa, Homo sapiens
MAAYNCAGEGQTAMVTFR

SEQ ID NO: 1250 - protein, 16 aa, Homo sapiens
CYFPYLENGYNQNYGR

SEQ ID NO: 1251 - protein, 9 aa, Homo sapiens
ERLIEEGVD

SEQ ID NO: 1252 - protein, 13 aa, Homo sapiens
EGTCPEAPTDECK

SEQ ID NO: 1253 - protein, 11 aa, Homo sapiens
ACEPGVDYVYK

SEQ ID NO: 1254 - protein, 15 aa, Homo sapiens
TGPGGDKGDTGPPGP

SEQ ID NO: 1255 - protein, 8 aa, Homo sapiens
GQAQVMNG

SEQ ID NO: 1256 - protein, 13 aa, Homo sapiens
SPGERGETGPPGP

SEQ ID NO: 1257 - protein, 12 aa, Homo sapiens
NDVNPVSLQLDL

SEQ ID NO: 1258 - protein, 6 aa, Homo sapiens
ALLLGR

SEQ ID NO: 1259 - protein, 18 aa, Homo sapiens
TGSPGSPGPDGKTGPPGP

SEQ ID NO: 1260 - protein, 9 aa, Homo sapiens
YTDVNGEKV

SEQ ID NO: 1261 - protein, 13 aa, Homo sapiens
SGSVVPDLPVFLP

SEQ ID NO: 1262 - protein, 19 aa, Homo sapiens
QKQQQQQQQGHKAPAAHPE

SEQ ID NO: 1263 - protein, 9 aa, Homo sapiens
IMLHPAPPN

SEQ ID NO: 1264 - protein, 19 aa, Homo sapiens
FAELQTDIHELTNDLDGAG

SEQ ID NO: 1265 - protein, 12 aa, Homo sapiens
ESTECPCVHSGK

SEQ ID NO: 1266 - protein, 8 aa, Homo sapiens
KSAVGELS

SEQ ID NO: 1267 - protein, 10 aa, Homo sapiens
NDESTEGKTS

SEQ ID NO: 1268 - protein, 15 aa, Homo sapiens
VGFYESDVMGRGHAR

SEQ ID NO: 1269 - protein, 11 aa, Homo sapiens
PSGEVSHPRKT

SEQ ID NO: 1270 - protein, 20 aa, Homo sapiens
ENLPQNGRQCYSCKGNSTHG

SEQ ID NO: 1271 - protein, 24 aa, Homo sapiens
ASGYFHTLRAVMRNPEEDGKDTLQ

SEQ ID NO: 1272 - protein, 9 aa, Homo sapiens
QRDSAFPKF

SEQ ID NO: 1273 - protein, 15 aa, Homo sapiens
LVALGSGVPSPCPVF

SEQ ID NO: 1274 - protein, 16 aa, Homo sapiens
QGVNDNEEGFFSARGH

SEQ ID NO: 1275 - protein, 7 aa, Homo sapiens
SGSTGNI

SEQ ID NO: 1276 - protein, 13 aa, Homo sapiens
APGDRGEPGPPGP

SEQ ID NO: 1277 - protein, 13 aa, Homo sapiens
DPGLPGAPGERGI

SEQ ID NO: 1278 - protein, 13 aa, Homo sapiens
NGFKSHALQLNNR

SEQ ID NO: 1279 - protein, 12 aa, Homo sapiens
LQRFLKPQDIEI

SEQ ID NO: 1280 - protein, 17 aa, Homo sapiens
HPSAQNGLFNMTLVSPS

SEQ ID NO: 1281 - protein, 10 aa, Homo sapiens
HQAAEGQLQA

SEQ ID NO: 1282 - protein, 10 aa, Homo sapiens
NCPPAVTENK

SEQ ID NO: 1283 - protein, 13 aa, Homo sapiens
SKIMTALKEDAQV

SEQ ID NO: 1284 - protein, 18 aa, Homo sapiens
SPGEAGRPGEAGLPGAKG

SEQ ID NO: 1285 - protein, 22 aa, Homo sapiens
RSTATQHSTATQSSTATQTSPV

SEQ ID NO: 1286 - protein, 6 aa, Homo sapiens
ANPAQR

SEQ ID NO: 1287 - protein, 12 aa, Homo sapiens
ALQGSAPRESPL

SEQ ID NO: 1288 - protein, 17 aa, Homo sapiens
HRHPDEAAFFDTASTGK

SEQ ID NO: 1289 - protein, 9 aa, Homo sapiens
SHALQLNNR

SEQ ID NO: 1290 - protein, 10 aa, Homo sapiens
APAVAEENPK

SEQ ID NO: 1291 - protein, 15 aa, Homo sapiens
GPKGEKGEPGSIFSP

SEQ ID NO: 1292 - protein, 13 aa, Homo sapiens
SEELDRTPPEVSK

SEQ ID NO: 1293 - protein, 21 aa, Homo sapiens
RDPSGQASATCNKALKPEDAK

SEQ ID NO: 1294 - protein, 28 aa, Homo sapiens
NPDGSPAYRVPVAVQGEDTVQSLTQGDG

SEQ ID NO: 1295 - protein, 27 aa, Homo sapiens
ELPGLCQGGDCVNTFGSFQCECPPGYH

SEQ ID NO: 1296 - protein, 39 aa, Homo sapiens
GSAGHWTSESSVSGSTGQWHSESGSFRPDSPGSGNARPN

SEQ ID NO: 1297 - protein, 9 aa, Homo sapiens
QNVSKTQVG

SEQ ID NO: 1298 - protein, 7 aa, Homo sapiens
GNSISSK

SEQ ID NO: 1299 - protein, 9 aa, Homo sapiens
SCAAGFGQR

SEQ ID NO: 1300 - protein, 9 aa, Homo sapiens
FVPAGESSS

SEQ ID NO: 1301 - protein, 14 aa, Homo sapiens
NNSQIASGQNQPQA

SEQ ID NO: 1302 - protein, 32 aa, Homo sapiens
GFSDPSQQPPSYGGPSVPGSGGPPAGGSGFGR

SEQ ID NO: 1303 - protein, 11 aa, Homo sapiens
QGKIPEELSLD

SEQ ID NO: 1304 - protein, 13 aa, Homo sapiens
VSFGPLPSPGSPS

SEQ ID NO: 1305 - protein, 21 aa, Homo sapiens
GTRCAGEVAASANNSYCIVGI

SEQ ID NO: 1306 - protein, 16 aa, Homo sapiens
NNEGTYYSPNYNPQSR

SEQ ID NO: 1307 - protein, 16 aa, Homo sapiens
GLPGTGGPPGENGKPG

SEQ ID NO: 1308 - protein, 19 aa, Homo sapiens
RQPDSTFDPEYPGPPRTTL

SEQ ID NO: 1309 - protein, 8 aa, Homo sapiens
HSAVPDSL

SEQ ID NO: 1310 - protein, 20 aa, Homo sapiens
EPCSCGPSSCFVRWAAMSLI

SEQ ID NO: 1311 - protein, 20 aa, Homo sapiens
GQGAFVRSQTETKVEPFEVP

SEQ ID NO: 1312 - protein, 26 aa, Homo sapiens
DKGAIGMPGRVGQPGTRGFPGFPGPI

SEQ ID NO: 1313 - protein, 20 aa, Homo sapiens
QIGGDGMMDITDTYKFQEGQ

SEQ ID NO: 1314 - protein, 25 aa, Homo sapiens
ITCRASQGISNGLSWYQQKPGQAPT

SEQ ID NO: 1315 - protein, 23 aa, Homo sapiens
SPNMGMGTSGPNQGPTQSTGMMN

SEQ ID NO: 1316 - protein, 19 aa, Homo sapiens
QLKGGEVTCLTLNSLGIQM

SEQ ID NO: 1317 - protein, 31 aa, Homo sapiens
PPQQLEASLRMLSLSATLPPAATTDQDKSEA

SEQ ID NO: 1318 - protein, 11 aa, Homo sapiens
HWSTKPPICQR

SEQ ID NO: 1319 - protein, 17 aa, Homo sapiens
GSPGSPGPDGKTGPPGP

SEQ ID NO: 1320 - protein, 17 aa, Homo sapiens
KFLQEQLNIQKDSLQAR

SEQ ID NO: 1321 - protein, 14 aa, Homo sapiens
GRCIPGAWQCDGLP

SEQ ID NO: 1322 - protein, 9 aa, Homo sapiens
LQPSGCHEK

SEQ ID NO: 1323 - protein, 13 aa, Homo sapiens
FSALEVDETYVPK

SEQ ID NO: 1324 - protein, 13 aa, Homo sapiens
LCNTGDNSVCTTK

SEQ ID NO: 1325 - protein, 14 aa, Homo sapiens
SAVVPEHGQKTPRK

(19) United States
(12) Patent Application Publication
(10) Pub. No.: US 2007/0292869 A1
(43) Pub. Date: Dec. 20, 2007

(54) Compositions and Methods for Analyzing Renal Cancer

(75) Inventors: Christopher H. Becker, Palo Alto, CA (US); Peter E. Clark, Franklin, TN (US); Theodore W. Jones, San Mateo, CA (US); Shanhua Lin, Palo Alto, CA (US); Thomas A. Shaler, Fremont, CA (US); Frank Torti, Winston-Salem, NC (US)

Correspondence Address: SHERIDAN ROSS PC, 1560 BROADWAY, SUITE 1200, DENVER, CO, 80202, US

(73) Assignee: PPD BIOMARKER DISCOVERY SCIENCES, LLC, Wilmington, NC (US)

(21) Appl. No.: 11/681,305

(22) Filed: Mar. 2, 2007

Related U.S. Application Data
(60) Provisional application No. 60/779,328, filed on Mar. 2, 2006.

(51) Int. Cl. G01N 33/574; C12Q 1/68; G01N 33/566

(52) U.S. Cl. 435/6; 436/501

(57) ABSTRACT

Compositions, methods and kits useful for the diagnosis, prognosis, and treatment of renal cell cancer are provided.

### CROSS-REFERENCE TO RELATED APPLICATION

This application claims the benefit of U.S. Provisional Application No. 60/779,328, filed Mar. 2, 2006.

### FIELD OF THE INVENTION

The present invention provides compositions, methods and kits useful for the diagnosis, prognosis, and treatment of renal cell cancer. In particular, the invention provides polypeptides that are markers of renal cell cancer, polynucleotides that encode the polypeptides and antibodies and aptamers that specifically bind to the polypeptides. The invention also provides fragments, precursors, successors and modified versions of the foregoing polypeptides, polynucleotides, antibodies and aptamers. The invention also provides compositions comprising the foregoing polypeptides, polynucleotides, antibodies, and aptamers. The invention also provides methods for using the polypeptides, polynucleotides, aptamers and antibodies in the diagnosis and treatment of renal cell cancer, monitoring progression of the disease and screening of candidate therapeutic compounds.

### BACKGROUND OF THE INVENTION

Renal cell carcinoma accounts for approximately 3% of adult malignancies and 90-95% of neoplasms arising from the kidney. In general, it is a highly treatment-resistant tumor type. Renal clear-cell carcinoma (also known as conventional or nonpapillary), is the most common type of renal cell carcinoma accounting for 70-80% of all cases. In this cancer, mutations to the von Hippel-Lindau (VHL) gene results in the up regulation of many proteins necessary for tumor growth and survival; however, it is believed that multiple pathways contribute to tumor growth. Many of these kidney tumors go undetected as they are generally asymptomatic. Often, they are detected only during ultrasound, computerized tomography, or magnetic resonance imaging procedures undertaken for unrelated reasons. Although advances in treatment and diagnosis have been made recently, the incidence of this disease continues to increase. See Pantuck, et al., Clin Cancer Res. 9:4641-4652 (2003). An unmet need therefore exists for biochemical markers of renal clear-cell cancer that will improve the diagnosis, prognosis, allow an early detection of disease and improve monitoring or design of therapeutics for the disease.

### SUMMARY OF THE INVENTION

One aspect of the invention provides polypeptides (“polypeptide markers”) that have been identified as differentially expressed in renal cell cancer serum samples, including plasma proteins from patients diagnosed with renal cell cancer, as compared samples from same patients who were cancer-free after surgery to remove the diseased kidney. The invention also provides polypeptides that have substantial sequence identity to polypeptide markers, modified polypeptide markers, and fragments of the polypeptide markers. The invention also includes precursors and successors of the polypeptide markers in biological pathways. The invention also provides molecules that comprise a polypeptide marker, homologous polypeptides, a modified polypeptide marker or a fragment thereof, precursor or successor of a polypeptide marker (e.g., a fusion protein). In some embodiments, the renal cell cancer is renal clear-cell cancer. As used herein, the term “polypeptides of the invention” shall be understood to include all of the foregoing.

Another aspect of the invention provides polynucleotides encoding polypeptides of the invention (“polynucleotide markers”). The invention also provides polynucleotides that have substantial sequence identity to polynucleotide markers, modified polynucleotide markers, and fragments of polynucleotide markers. The invention also provides molecules that comprise a polynucleotide marker, a homologous polynucleotide, a modified polynucleotide marker or a fragment of a polynucleotide marker (e.g., a vector). As used herein, the term “polynucleotides of the invention” shall be understood to include all of the foregoing.

Another aspect of the invention provides molecules that specifically bind to a polypeptide of the invention or polynucleotide of the invention. The binding molecule may be an antibody, antibody fragment, apatmer, or other molecule. The invention also provides methods for producing a binding molecule that specifically recognizes a polypeptide of the invention or polynucleotide of the invention.

Another aspect of the invention provides compositions comprising a polypeptide of the invention or polynucleotide of the invention, a binding molecule (e.g., an antibody or aptamer) that is specific for a polypeptide of the invention or polypeptide of the invention, an inhibitor of a polypeptide of the invention or polynucleotide of the invention, or another molecule that can increase or decrease the level or activity of a polypeptide of the invention or polynucleotide of the invention. Such compositions may be pharmaceutical compositions formulated for use as therapeutics.

Another aspect of the invention provides a method for detecting a polypeptide of the invention or polynucleotide of the invention. In one embodiment, the method comprises contacting a biological sample obtained from a subject with a binding molecule (e.g., an antibody or aptamer) under conditions that permit the formation of a stable complex, and detecting any stable complexes formed. In another embodiment, the method comprises determining the activity of a polypeptide of the invention or polynucleotide of the invention. In another embodiment, the method comprises determining the level of a polypeptide of the invention in a cell obtained from the subject by detecting the presence of a polynucleotide that encodes the polypeptide.

Another aspect of the invention provides a method for diagnosing renal cell cancer in a subject by detecting a polypeptide of the invention or polynucleotide of the invention in a biological sample. In one embodiment, the method comprises obtaining a sample from a subject suspected of having renal clear-cell cancer or at risk for renal clear-cell cancer and comparing the level or activity of a polypeptide of the invention or polynucleotide of the invention in the sample with the level of activity in a sample obtained from a non-renal clear-cell cancer subject or with a reference range or value. In some embodiments, renal clear-cell cancer is diagnosed in the patient if the expression level of the biomarker or biomarkers in the patient sample is statistically more similar to the expression level of the biomarker or biomarkers that has been associated with renal clear-cell cancer than the expression level of the biomarker or biomarkers that has been associated with the normal controls. In some embodiments, the method is used for staging or stratifying subjects with renal clear-cell cancer, monitoring the progression of the disease or response to therapy. In some embodiments, a plurality of polypeptides of the invention or polynucleotides of the invention are detected. In some embodiments, the method comprises detecting known biomarkers or considering other clinical indicia in addition to detecting one or more polypeptides of the invention or polynucleotides of the invention in a biological sample.

Another aspect of the invention provides methods for treating renal clear-cell cancer by administering a therapeutic agent to a subject that increases or decreases the level or activity of a polypeptide of the invention or polynucleotide of the invention. For polypeptides of the invention or polynucleotides of the invention that are increased in samples obtained from a renal clear-cell cancer subject, the method comprises administering a therapeutic agent that decreases (i.e., bring toward the normal range) the level or activity of the polypeptide or polynucleotide. Similarly, for polypeptides of the invention or polynucleotides of the invention that are decreased in samples obtained from a renal clear-cell cancer subject, the method comprises administering a therapeutic agent that increases the level or activity of the polypeptide or polynucleotide.

Another aspect of the present invention provides a method for screening a candidate compound for use as a therapeutic agent for treating renal clear-cell cancer. In one embodiment, the method comprises administering the candidate compound to a renal clear-cell cancer subject and screening for the ability to modulate the level or activity of a polypeptide of the invention or polynucleotide of the invention. In another embodiment, the method comprises providing the candidate compound to a cell from a renal clear-cell cancer subject and screening for the ability to modulate the intracellular level of a polypeptide of the invention or polynucleotide of the invention.

Another aspect of the invention provides a kit for performing the methods described above. In one embodiment, the kit is for the diagnosis of renal clear-cell cancer by detection of a polypeptide of the invention or polynucleotide of the invention in a biological sample from a subject. A kit for detecting a polypeptide of the invention or polynucleotide of the present invention may include an antibody capable of binding to the polypeptide or polynucleotide.

Another aspect of the invention includes the use of animal models of renal carcinoma. For example, the markers identified in the present application can be used in research aimed to discover and/or test biomarkers with relevance in humans.

Other features and advantages of the invention will become apparent to one of skill in the art from the following detailed description, including Tables 1-2, and from the claims.

### DETAILED DESCRIPTION OF THE INVENTION

The terminology used herein is for describing particular embodiments and is not intended to be limiting. As used herein, the singular forms “a,” “and” and “the” include plural referents unless the content and context clearly dictate otherwise. Thus, for example, a reference to “a marker” includes a combination of two or more such markers. Unless defined otherwise, all scientific and technical terms are to be understood as having the same meaning as commonly used in the art to which they pertain. For the purposes of the present invention, the following terms are defined below.

The invention generally relates to the identification of a large number of polypeptides and related molecules that are differentially expressed in serum in patients with renal cell cancer compared to serum samples in patients without renal cell cancer (due to surgical removal of the carcinoma). In some embodiments, renal cell cancer is renal clear-cell cancer.

As used herein, the term “marker” includes polypeptide markers and polynucleotide markers. For clarity of disclosure, aspects of the invention will be described with respect to “polypeptide markers” and “polynucleotide markers.” However, statements made herein with respect to “polypeptide markers” are intended to apply to other polypeptides of the invention. Likewise, statements made herein with respect to “polynucleotide” markers are intended to apply to other polynucleotides of the invention, respectively. Thus, for example, a polynucleotide described as encoding a “polypeptide marker” is intended to include a polynucleotide that encodes: a polypeptide marker, a polypeptide that has substantial sequence identity to a polypeptide marker, modified polypeptide markers, fragments of a polypeptide marker, precursors of a polypeptide marker and successors of a polypeptide marker, and molecules that comprise a polypeptide marker, homologous polypeptide, a modified polypeptide marker or a fragment, precursor or successor of a polypeptide marker (e.g., a fusion protein).

As used herein, the term “polypeptide” refers to a polymer of amino acid residues that has at least 5 contiguous amino acid residues, e.g., 5, 6, 7, 8, 9, 10, 11 or 12 or more amino acids long, including each integer up to the full length of the polypeptide. A polypeptide may be composed of two or more polypeptide chains. A polypeptide includes a protein, a peptide, an oligopeptide, and an amino acid. A polypeptide can be linear or branched. A polypeptide can comprise modified amino acid residues, amino acid analogs or non-naturally occurring amino acid residues and can be interrupted by non-amino acid residues. Included within the definition are amino acid polymers that have been modified, whether naturally or by intervention, e.g., formation of a disulfide bond, glycosylation, lipidation, methylation, acetylation, phosphorylation, or by manipulation, such as conjugation with a labeling component. Also included are antibodies produced by a subject in response to overexpressed polypeptide markers.

As used herein, a “fragment” of a polypeptide refers to a single amino acid or a plurality of amino acid residues comprising an amino acid sequence that has at least 5 contiguous amino acid residues, at least 10 contiguous amino acid residues, at least 20 contiguous amino acid residues or at least 30 contiguous amino acid residues of a sequence of the polypeptide. As used herein, a “fragment” of polynucleotide refers to a single nucleic acid or to a polymer of nucleic acid residues comprising a nucleic acid sequence that has at least 15 contiguous nucleic acid residues, at least 30 contiguous nucleic acid residues, at least 60 contiguous nucleic acid residues, or at least 90% of a sequence of the polynucleotide. In some embodiment, the fragment is an antigenic fragment, and the size of the fragment will depend upon factors such as whether the epitope recognized by an antibody is a linear epitope or a conformational epitope. Thus, some antigenic fragments will consist of longer segments while others will consist of shorter segments, (e.g. 5, 6, 7, 8, 9, 10, 11 or 12 or more amino acids long, including each integer up to the full length of the polypeptide). Those skilled in the art are well versed in methods for selecting antigenic fragments of proteins.

In some embodiments, a polypeptide marker is a member of a biological pathway. As used herein, the term “precursor” or “successor” refers to molecules that precede or follow the polypeptide marker or polynucleotide marker in the biological pathway. Thus, once a polypeptide marker or polynucleotide marker is identified as a member of one or more biological pathways, the present invention can include additional precursor or successor members of the biological pathway. Such identification of biological pathways and their members is within the skill of one in the art.

As used herein, the term “polynucleotide” refers to a single nucleotide or a polymer of nucleic acid residues of any length. The polynucleotide may contain deoxyribonucleotides, ribonucleotides, and/or their analogs and may be double-stranded or single stranded. A polynucleotide can comprise modified nucleic acids (e.g., methylated), nucleic acid analogs or non-naturally occurring nucleic acids and can be interrupted by non-nucleic acid residues. For example a polynucleotide includes a gene, a gene fragment, cDNA, isolated DNA, mRNA, tRNA, rRNA, isolated RNA of any sequence, recombinant polynucleotides, primers, probes, plasmids, and vectors. Included within the definition are nucleic acid polymers that have been modified, whether naturally or by intervention.

As used herein, a component (e.g., a marker) is referred to as “differentially expressed” in one sample as compared to another sample when the method used for detecting the component provides a different level or activity when applied to the two samples. A component is referred to as “increased” in the first sample if the method for detecting the component indicates that the level or activity of the component is higher in the first sample than in the second sample (or if the component is detectable in the first sample but not in the second sample). Conversely, a component is referred to as “decreased” in the first sample if the method for detecting the component indicates that the level or activity of the component is lower in the first sample than in the second sample (or if the component is detectable in the second sample but not in the first sample). In particular, marker is referred to as “increased” or “decreased” in a sample (or set of samples) obtained from a renal clear-cell cancer subject (or a subject who is suspected of having renal clear-cell cancer, or is at risk of developing renal clear-cell cancer) if the level or activity of the marker is higher or lower, respectively, compared to the level of the marker in a sample (or set of samples) obtained from a non-renal clear-cell cancer subject, or a reference value or range.

The markers identified as being differentially expressed in renal clear-cell cancer vs. normal controls (see Examples) are of significant biologic interest. Briefly, serum samples were obtained from patients with renal clear-cell cancer and from the same patients without renal clear-cell cancer as a result of surgery to remove the diseased kidney. All samples were separated into a high molecular weight fraction, containing proteins with molecular weights greater than about 5-kDa, and a low molecular weight fraction containing free floating peptides and small molecules having a molecular weight of less than about 5-kDa. After removal of high abundance proteins, the high molecular weight fraction was digested with trypsin. Each fraction was separated by chromatographic means and analyzed by mass spectrometry. The resulting spectra were compared to identify individual markers that showed significant association with renal clear-cell cancer.

In addition to the discovery of biomarkers that can be used individually or in any combination in assays and kits for the diagnosis of, prognosis of, or other evaluation or study of renal clear-cell cancer, the biomarkers not previously recognized to play a role in the disease process of renal clear-cell cancer can now be studied in more detail and/or be used as targets for the discovery of other modulators of disease or therapeutic agents. Tables 1-2 provide polypeptide markers that were found at significantly different levels in serum samples obtained from patients with renal clear-cell cancer. Polypeptides found in the high molecular weight fraction (“plasma proteome,” see Example 1) are shown in Table 1: Renal Clear-Cell Cancer Proteome Study: Diseased vs. Control. Table 1 shows a component-level view of the molecules tracked with p<0.05 or CountDiffmin of +/−8 (see the definition of CountDiffmin below). Polypeptides found in the lower molecular-weight polypeptide fraction (“plasma peptidome,” see Example 2) are shown in Table 2: Renal Clear-Cell Cancer Peptidome Study: Diseased vs. Control. Table 2 shows a component-level view of the molecules tracked with p<0.05 or CountDiffmin of +/−8 (see the definition of CountDiffmin below).

The abbreviations used in the Tables will be familiar to those of skill in the art. For clarity, “Comp. #” refers to the component number; “m/z” refers to the mass-to-charge ratio; “R.T. (min)” refers to the retention time in minutes; RI is Retention Index with is conversion from time to an index so that our observations “z” refers to the charge; “M+H” refers to the protonated molecular ion mass; “gi #” refers to the GenInfo Identifier; “Exp. Ratio” refers to the expression ratio, which is a ratio of mean group intensities indicating the relative normalized signal for disease group compared to control; “Mods” refers to modifications; “DM(mD)” refers to difference in mass in milliDalton between observed and predicted values; “DM(ppm)” refers to difference in mass in parts per million between observed and predicted values; fold change (an expression change factor where positive indicates a relative intensity increase and negative indicates a relative decrease versus the control); “CountDiff” refers to the count difference between study groups or the difference between two study groups of the number of subjects reporting a detectable intensity for a given component; CountDiffmin refers to the minimum number by which two groups may differ in count, to be categorized as a CountDiff, and therefore to be considered as significantly differentially expressed; and where available, identification number from NCBI's reference sequence database (Accession # and gi #) and additional information (e.g., the name or sequence of the peptide marker as contained in the NCBI queried database and database searching using the Mascot or TurboSEQUEST programs). All information associated with the publicly available identifiers and accession numbers in any of the tables described herein, including the nucleic acid sequences of the associated genes, is incorporated herein by reference in its entirety. Given the name of the protein (also referred to herein as the “full protein”; indicated as “Protein”), other peptide fragments of such measured proteins may be obtained (by whatever means), and such other peptide fragments are included within the scope of the invention. The methods of the present invention may be used to evaluate fragments of the listed molecules as well as molecules that contain an entire listed molecule, or at least a significant portion thereof (e.g., measured unique epitope), and modified versions of the markers. Accordingly, such fragments, larger molecules and modified versions are included within the scope of the invention.

As one of skill in the art will appreciate, the physical and chemical properties presented in the Tables are sufficient to distinguish the component from other materials. In some embodiments, the markers set forth in the Tables 1-2 are each identified on the mass to charge ratio (m/z), chromatographic retention time (RT), the charge state of a molecular ion (z), protonated parent mass (M+H), and expression ratio (exp. ratio). In other embodiments, the components are uniquely identified by the mass to charge ratio (m/z) and the retention time (RT).

Homologs and alleles of the polypeptide markers of the invention can be identified by conventional techniques. As used herein, a homolog to a polypeptide is a polypeptide from a human or other animal that has a high degree of structural similarity to the identified polypeptides. Identification of human and other organism homologs of polypeptide markers identified herein will be familiar to those of skill in the art. In general, nucleic acid hybridization is a suitable method for identification of homologous sequences of another species (e.g., human, cow, sheep), which correspond to a known sequence. Standard nucleic acid hybridization procedures can be used to identify related nucleic acid sequences of selected percent identity. For example, one can construct a library of cDNAs reverse transcribed from the mRNA of a selected tissue (e.g., colon) and use the nucleic acids that encode polypeptides identified herein to screen the library for related nucleotide sequences. The screening preferably is performed using high-stringency conditions (described elsewhere herein) to identify those sequences that are closely related by sequence identity. Nucleic acids so identified can be translated into polypeptides and the polypeptides can be tested for activity.

Many of the polypeptides listed in Tables 1-2 are fragments of complete proteins (“parent proteins”), either because they were present as fragments in the sample or as a result of the trypsin digestion that was performed during the processing of certain fractions of the sample (see Example). The parent proteins are included as polypeptide markers. In many cases, the sequence of the parent protein can be ascertained from the amino acid sequence of the fragment by searching a protein sequence database. The tables of the invention include the identification of proteins that include an identified polypeptide marker, although proteins comprising such polypeptides are not limited to those provided in the tables.

Additionally, the present invention includes polypeptides that have substantially similar sequence identity to the polypeptides of the present invention. As used herein, two polypeptides have “substantial sequence identity” when there is at least about 70% sequence identity, at least about 80% sequence identity, at least about 90% sequence identity, at least about 95% sequence identity or at least about 99% sequence identity between their amino acid sequences, or when polynucleotides encoding the polypeptides are capable of forming a stable duplex with each other under stringent hybridization conditions. For example, conservative amino acid substitutions may be made in polypeptides to provide functionally equivalent variants of the foregoing polypeptides, i.e., the variants retain the functional capabilities of the polypeptides. As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references that compile such methods. For example, upon determining that a peptide is a renal clear-cell cancer-associated polypeptide, one can make conservative amino acid substitutions to the amino acid sequence of the peptide, and still have the polypeptide retain its specific antibody-binding characteristics. Additionally, one skilled in the art will realize that allelic variants and SNPs will give rise to substantially similar polypeptides and the same or substantially similar polypeptide fragments.

A number of comparison studies were performed to identify the polypeptide markers listed using various groups of renal clear-cell cancer and non-renal clear-cell cancer patients. The Tables list markers that were found to be differentially present with statistical significance. Accordingly, it is believed that these biomarkers are indicators of renal clear-cell cancer. Where a polypeptide marker was found to be statistically significant in a plurality of studies, the data associated with the observations of highest statistical significance is presented. Accordingly, in one aspect, the invention provides polypeptides biomarkers of renal clear-cell cancer. In one embodiment, the invention provides an isolated component described in Tables 1-2. In another embodiment, the invention provides a polypeptide having substantial sequence identity with a component set forth in Tables 1-2. In another embodiment, the invention provides a molecule that comprises a foregoing polypeptide. As used herein, a compound is referred to as “isolated” when it has been separated from at least one component with which it is naturally associated. For example, a polypeptide can be considered isolated if it is separated from contaminants including metabolites, polynucleotides and other polypeptides. Isolated molecules can be either prepared synthetically or purified from their natural environment. Standard quantification methodologies known in the art can be employed to obtain and isolate the molecules of the invention.

Some variation is inherent in the measurements of the physical and chemical characteristics of the markers. The magnitude of the variation depends to some extent on the reproductively of the separation means and the specificity and sensitivity of the detection means used to make the measurement. Preferably, the method and technique used to measure the markers is sensitive and reproducible.

The retention time and mass to charge ratio may vary to some extent depending on a number of factors relating to the protocol used for the chromatography and the mass spectrometry parameters (e.g., solvent composition, flow rate). Preferably, sample preparation and analysis conditions are carefully controlled. However, one of skill in the art will appreciate that the possibility of contamination or measurement of artifacts can never be completely eliminated.

The data set forth in the Tables reflects the method that was used to detect the markers. When a sample is processed and analyzed as described in the Example, the retention time of the marker is about the value stated for the marker; that is, within about 10% of the value stated, within about 5% of the value stated, or within about 1% of the value stated, and the marker has a mass to charge ratio of about the value stated for the marker; that is, within about 10% of the value stated, within about 5% of the value stated, or within about 1% of the value stated. Accordingly, in another embodiment, the invention provides a polypeptide having (i) a mass-to-charge value and (ii) an RT value of about the values stated, respectively, for a component described in Tables 1-2. In another embodiment, the invention provides a molecule that comprises a foregoing polypeptide.

Polypeptide identifications in Tables 1-2 reflect a single polypeptide appearing in a database for which the component was a match. In general, the polypeptide is the largest polypeptide found in the database. Such a selection is not meant to limit the polypeptide to those disclosed in Tables 1-2, however. Accordingly, in another embodiment, the invention provides a polypeptide that is a fragment, precursor, successor or modified version of a marker described in Tables 1-2. For example the following polypeptides appear in Table 1: C4b-binding protein beta chain precursor, coagulation factor XII precursor (Hageman factor) (HAF), and apolipoprotein A-IV precursor [validated]-human. Such precursors are typically larger than the processed form. The invention therefore includes the successor molecules (i.e., processed proteins) C4b-binding protein beta chain, coagulation factor XII, and apolipoprotein A-IV. In another embodiment, the invention includes a molecule that comprises a foregoing fragment, precursor, successor or modified polypeptide.

Another embodiment of the present invention relates to a plurality of antibodies, or antigen binding fragments thereof, or aptamers for the detection of the expression of biomarkers differentially expressed in patients with renal clear-cell cancer. The plurality of antibodies, or antigen binding fragments thereof, or aptamers consists of antibodies, or antigen binding fragments thereof, or aptamers that selectively bind to proteins differentially expressed in patients with renal clear-cell cancer, and that can be detected as protein products using antibodies or aptamers. In addition, the plurality of antibodies, or antigen binding fragments thereof, or aptamers comprises antibodies, or antigen binding fragments thereof, or aptamers that selectively bind to proteins or portions thereof (peptides) encoded by any of the genes from the tables provided herein.

Certain embodiments of the present invention utilize a plurality of biomarkers that have been identified herein as being differentially expressed in subjects with renal clear-cell cancer. As used herein, the terms “patient,” “subject” and “a subject who has renal clear-cell cancer” and “renal clear-cell cancer subject” are intended to refer to subjects who have been diagnosed with renal clear-cell cancer. The terms “non-subject” and “a subject who does not have renal clear-cell cancer” are intended to refer to a subject who has not been diagnosed with renal clear-cell cancer, or who is cancer-free as a result of surgery to remove the diseased kidney. A non-renal clear-cell cancer subject may be healthy and have no other disease, or they may have a disease other than renal clear-cell cancer.

The plurality of biomarkers within the above-limitation includes at least two or more biomarkers (e.g., at least 2, 3, 4, 5, 6, and so on, in whole integer increments, up to all of the possible biomarkers) identified by the present invention, and includes any combination of such biomarkers. Such biomarkers are selected from any of the polypeptides listed in the tables provided herein, and polynucleotides encoding any of the polypeptides listed in the Tables.

The polypeptide and polynucleotide markers of the invention are useful in methods for diagnosing renal clear-cell cancer, determining the extent and/or severity of the disease, monitoring progression of the disease and/or response to therapy. Such methods can be performed in human and non-human subjects. The markers are also useful in methods for treating renal clear-cell cancer and for evaluating the efficacy of treatment for the disease. Such methods can be performed in human and non-human subjects. The markers may also be used as pharmaceutical compositions or in kits. The markers may also be used to screen candidate compounds that modulate their expression. The markers may also be used to screen candidate drugs for treatment of renal clear-cell cancer. Such screening methods can be performed in human and non-human subjects.

Polypeptide markers may be isolated by any suitable method known in the art. Native polypeptide markers can be purified from natural sources by standard methods known in the art (e.g., chromatography, centrifugation, differential solubility, immunoassay). In one embodiment, polypeptide markers may be isolated from a serum sample using the chromatographic methods disclosed herein. In another embodiment, polypeptide markers may be isolated from a sample by contacting the sample with substrate-bound antibodies or aptamers that specifically bind to the marker.

The present invention also included polynucleotide markers related to the polypeptide markers of the present invention. In one aspect, the invention provides polynucleotides that encode the polypeptides of the invention. The polynucleotide may be genomic DNA, cDNA, or mRNA transcripts that encode the polypeptides of the invention. In one embodiment, the invention provides polynucleotides that encode a polypeptide described in Tables 1-2, or a molecule that comprises such a polypeptide.

In another embodiment, the invention provides polynucleotides that encode a polypeptide having substantial sequence identity with a component set forth in Tables 1-2, or a molecule that comprises such a polypeptide.

In another embodiment, the invention provides polynucleotides that encode a polypeptide having (i) a mass-to-charge value and (ii) an RT value of about the values stated, respectively, for a marker described in Tables 1-2, or a molecule that comprises such a polypeptide.

In another embodiment, the invention provides polynucleotides that encode a polypeptide having (i) a mass-to-charge value within 10% (more particularly within 5%, more particularly within 1%) and (ii) an RT value within 10% (more particularly within 5%, more particularly within 1%) of the m/z and RT values stated, respectively, for a component described in Tables 1-2, or a molecule that comprises such polypeptide.

In another embodiment, the invention provides polynucleotides that encode a polypeptide that is a fragment, precursor, successor or modified version of a marker described in Tables 1-2, or a molecule that comprises such polypeptide.

In another embodiment, the invention provides polynucleotides that have substantial sequence similarity to a polynucleotide that encodes a polypeptide that is a fragment, precursor, successor or modified version of a marker described in Tables 1-2, or a molecule that comprises such polypeptide. Two polynucleotides have “substantial sequence identity” when there is at least about 70% sequence identity, at least about 80% sequence identity, at least about 90% sequence identity, at least about 95% sequence identity or at least 99% sequence identity between their amino acid sequences or when the polynucleotides are capable of forming a stable duplex with each other under stringent hybridization conditions. Such conditions are described elsewhere herein. As described above with respect to polypeptides, the invention includes polynucleotides that are allelic variants, the result of SNPs, or that in alternative codons to those present in the native materials as inherent in the degeneracy of the genetic code.

In some embodiments, the polynucleotides described may be used as surrogate markers of renal clear-cell cancer. Thus, for example, if the level of a polypeptide marker is increased in renal clear-cell cancer-patients, an increase in the mRNA that encodes the polypeptide marker may be interrogated rather than the polypeptide marker (e.g., to diagnose renal clear-cell cancer in a subject).

Polynucleotides encoding the polypeptides markers listed in Tables 1-2 can be used to screen existing genomic, cDNA or expression libraries to find the gene that encodes the polynucleotide of the invention. A library is typically screened using a probe that is complementary either to the polynucleotide that encodes a polypeptide in Tables 1-2, or to its complement, under conditions which promote hybridization, including stringent hybridization. Hybridization is monitored by any suitable method known in the art. Once located, the gene can be cloned. The protein product of a gene that encodes a fragment of a polynucleotide marker is also included as a polypeptide marker. Alternatively, the sequence of the polynucleotide that encode a polypeptide listed in Tables 1-2 can be used to search databases such as SWISS-PROT and NCBI's RefSeq database, which will provide the gene sequence(s) comprising the nucleic acid sequence, and the amino acid sequence of the gene product.

Polynucleotide markers may be isolated by any suitable method known in the art. Native polynucleotide markers may be purified from natural sources by standard methods known in the art (e.g., chromatography, centrifugation, differential solubility, immunoassay). In one embodiment, a polynucleotide marker may be isolated from a mixture by contacting the mixture with substrate bound probes that are complementary to the polynucleotide marker under hybridization conditions.

Alternatively, polynucleotide markers may be synthesized by any suitable chemical or recombinant method known in the art. In one embodiment, for example, the makers can be synthesized using the methods and techniques of organic chemistry. In another embodiment, a polynucleotide marker can be produced by polymerase chain reaction (PCR).

The present invention also encompasses molecules which specifically bind the polypeptide or polynucleotide markers of the present invention. In one aspect, the invention provides molecules that specifically bind to a polypeptide marker or a polynucleotide marker. As used herein, the term “specifically binding,” refers to the interaction between binding pairs (e.g., an antibody and an antigen or aptamer and its target). In some embodiments, the interaction has an affinity constant of at most 10⁻⁶ moles/liter, at most 10⁻⁷ moles/liter, or at most 10⁻⁸ moles/liter. In other embodiments, the phrase “specifically binds” refers to the specific binding of one protein to another (e.g., an antibody, fragment thereof, or binding partner to an antigen), wherein the level of binding, as measured by any standard assay (e.g., an immunoassay), is statistically significantly higher than the background control for the assay. For example, when performing an immunoassay, controls typically include a reaction well/tube that contain antibody or antigen binding fragment alone (i.e., in the absence of antigen), wherein an amount of reactivity (e.g., non-specific binding to the well) by the antibody or antigen binding fragment thereof in the absence of the antigen is considered to be background. Binding can be measured using a variety of methods standard in the art including enzyme immunoassays (e.g., ELISA), immunoblot assays, etc.).

The binding molecules include antibodies, aptamers and antibody fragments. As used herein, the term “antibody” refers to an immunoglobulin molecule capable of binding an epitope present on an antigen. The term is intended to encompasses not only intact immunoglobulin molecules such as monoclonal and polyclonal antibodies, but also bi-specific antibodies, humanized antibodies, chimeric antibodies, anti-idiopathic (anti-ID) antibodies, single-chain antibodies, Fab fragments, F(ab′) fragments, fusion proteins and any modifications of the foregoing that comprise an antigen recognition site of the required specificity. As used herein, an aptamer is a non-naturally occurring nucleic acid having a desirable action on a target. a desirable action includes, but is not limited to, binding of the target, catalytically changing the target, reacting with the target in a way which modifies/alters the target or the functional activity of the target, covalently attaching to the target as in a suicide inhibitor, facilitating the reaction between the target and another molecule. in the preferred embodiment, the action is specific binding affinity for a target molecule, such target molecule being a three dimensional chemical structure other than a polynucleotide that binds to the nucleic acid ligand through a mechanism which predominantly depends on Watson/Crick base pairing or triple helix binding, wherein the nucleic acid ligand is not a nucleic acid having the known physiological function of being bound by the target molecule.

In one aspect, the invention provides antibodies or aptamers that specifically bind to a component described in Tables 1-2, or to a molecule that comprises a foregoing component (e.g., a protein comprising a polypeptide identified in a table of the invention).

In another embodiment, the invention provides antibodies or aptamers that specifically bind to a polypeptide having substantial sequence identity with a component set forth in Tables 1-2, or to a molecule that comprises a foregoing polypeptide.

In another embodiment, the invention provides antibodies or aptamers that specifically bind to a component having (i) a mass-to-charge value and (ii) an RT value of about the values stated, respectively, for a marker described in Tables 1-2, or to a molecule that comprises a foregoing component.

In another embodiment, the invention provides antibodies or aptamers that specifically bind to a component having (i) a mass-to-charge value within 10% (more particularly within 5%, more particularly within 1%) and (ii) an RT value within 10% (more particularly within 5%, more particularly within 1%) of the m/z and RT values stated, respectively, for a component described in Tables 1-2, or to a molecule that comprises a foregoing component.

In another embodiment, the invention provides antibodies or aptamers that specifically bind to a component that is a fragment, modification, precursor or successor of a marker described in Tables 1-2, or to a molecule that comprises a foregoing component.

In another embodiment, the invention provides antibodies or aptamers that specifically bind to a polypeptide marker or a polynucleotide marker that is structurally different from a component specifically identified in Tables 1-2 but has the same (or nearly the same) function or properties, or to a molecule that comprises a foregoing component.

Another embodiment of the present invention relates to a plurality of aptamers, antibodies, or antigen binding fragments thereof, for the detection of the expression of biomarkers differentially expressed in patients with renal clear-cell cancer. The plurality of aptamers, antibodies, or antigen binding fragments thereof, consists of antibodies, or antigen binding fragments thereof, that selectively bind to proteins differentially expressed in patients with renal clear-cell cancer, and that can be detected as protein products using antibodies. In addition, the plurality of aptamers, antibodies, or antigen binding fragments thereof, comprises antibodies, or antigen binding fragments thereof, that selectively bind to proteins or portions thereof (peptides) encoded by any of the genes from the tables provided herein.

According to the present invention, a plurality of aptamers, antibodies, or antigen binding fragments thereof, refers to at least 2, and more preferably at least 3, and more preferably at least 4, and more preferably at least 5, and more preferably at least 6, and more preferably at least 7, and more preferably at least 8, and more preferably at least 9, and more preferably at least 10, and so on, in increments of one, up to any suitable number of antibodies, or antigen binding fragments thereof, including antibodies representing all of the biomarkers described herein, or antigen binding fragments thereof.

Certain antibodies that specifically bind polypeptide markers polynucleotide markers of the invention already may be known and/or available for purchase from commercial sources. In any event, the antibodies of the invention may be prepared by any suitable means known in the art. For example, antibodies may be prepared by immunizing an animal host with a marker or an immunogenic fragment thereof (conjugated to a carrier, if necessary). Adjuvants (e.g., Freund's adjuvant) optionally may be used to increase the immunological response. Sera containing polyclonal antibodies with high affinity for the antigenic determinant can then be isolated from the immunized animal and purified.

Alternatively, antibody-producing tissue from the immunized host can be harvested and a cellular homogenate prepared from the organ can be fused to cultured cancer cells. Hybrid cells which produce monoclonal antibodies specific for a marker can be selected. Alternatively, the antibodies of the invention can be produced by chemical synthesis or by recombinant expression. For example, a polynucleotide that encodes the antibody can be used to construct an expression vector for the production of the antibody. The antibodies of the present invention can also be generated using various phage display methods known in the art.

Antibodies or aptamers that specifically bind markers of the invention can be used, for example, in methods for detecting components described in Tables 1-2 using methods and techniques well-known in the art. In some embodiments, for example, the antibodies are conjugated to a detection molecule or moiety (e.g., a dye, and enzyme) and can be used in ELISA or sandwich assays to detect markers of the invention.

In another embodiment, antibodies or aptamers against a polypeptide marker or polynucleotide marker of the invention can be used to assay a tissue sample (e.g., a thin cortical slice) for the marker. The antibodies or aptamers can specifically bind to the marker, if any, present in the tissue sections and allow the localization of the marker in the tissue. Similarly, antibodies or aptamers labeled with a radioisotope may be used for in vivo imaging or treatment applications.

Another aspect of the invention provides compositions comprising a polypeptide or polynucleotide marker of the invention, a binding molecule that is specific for a polypeptide or polynucleotide marker (e.g., an antibody or an aptamer), an inhibitor of a polypeptide or polynucleotide marker, or other molecule that can increase or decrease the level or activity of a polypeptide marker or polynucleotide marker. Such compositions may be pharmaceutical compositions formulated for use as a therapeutic.

In one embodiment, the invention provides a composition that comprises a polypeptide or polynucleotide marker of the invention, such as a component described in Tables 1-2, a polypeptide having substantial sequence identity with a component or having (i) a mass-to-charge value and (ii) an RT value of about the values, respectively, for a component, or a molecule comprising such a component.

Alternatively, the invention provides a composition that comprises a component that is a fragment, modification, precursor or successor of a marker described in Tables 1-2, or to a molecule that comprises a foregoing component.

In another embodiment, the invention provides a composition that comprises a polynucleotide that binds to a polypeptide or a molecule that comprises a foregoing polynucleotide.

In another embodiment, the invention provides a composition that comprises an antibody or aptamer that specifically binds to a polypeptide or a molecule that comprises a foregoing antibody or aptamer.

In another embodiment, the invention provides a composition that comprises a modulator of the level or activity of a polypeptide marker (e.g., an inhibitor of a polypeptide marker, an antisense polynucleotide which is complementary to a polynucleotide that encodes a polypeptide marker), or a molecule that comprises a foregoing modulator.

Such compositions may be pharmaceutical compositions. Typically, a pharmaceutical composition comprises a therapeutically effective amount of an active agent and is formulated with a suitable excipient or carrier. The invention also provides pharmaceutical compositions for the treatment of renal clear-cell cancer. These compositions may include a marker protein and/or nucleic acid of the invention (e.g., for those markers which are decreased in quantity or activity in renal clear-cell cancer samples versus non-renal clear-cell cancer samples), and can be formulated as described herein. Alternately, these compositions may include an antibody which specifically binds to a marker protein of the invention and/or an antisense polynucleotide which is complementary to a polynucleotide marker of the invention (e.g., for those markers which are increased in quantity or activity in renal clear-cell cancer samples versus non-renal clear-cell cancer samples), and can be formulated as described herein.

The pharmaceutical compositions of the invention can be prepared in any suitable manner known in the pharmaceutical art. The carrier or excipient may be a solid, semisolid, or liquid material that can serve as a vehicle or medium for the active ingredient. Suitable carriers or excipients are well known in the art and include, but are not limited to saline, buffered saline, dextrose, water, glycerol, ethanol, and combinations thereof. The pharmaceutical compositions may be adapted for oral, inhalation, parenteral, or topical use and may be administered to the patient in the form of tablets, capsules, aerosols, inhalants, suppositories, solutions, suspensions, powders, syrups, and the like. As used herein, the term “pharmaceutical carrier” may encompass one or more excipients. In preparing formulations of the compounds of the invention, care should be taken to ensure bioavailability of an effective amount of the agent. Suitable pharmaceutical carriers and formulation techniques are found in standard texts, such as Remington's Pharmaceutical Sciences, Mack Publishing Co., Easton, Pa.

The present invention also provides methods of detecting the biomarkers of the present invention. The practice of the present invention employs, unless otherwise indicated, conventional methods of analytical biochemistry, microbiology, molecular biology and recombinant DNA techniques within the skill of the art. Such techniques are explained fully in the literature. (See, e.g., Sambrook, J. et al. Molecular Cloning: A Laboratory Manual. 3rd, ed., Cold Spring Harbor Laboratory, Cold Spring Harbor Laboratory Press, Cold Spring Harbor, N.Y., 2000; DNA Cloning: A Practical Approach, Vol. I & II (D. Glover, ed.); Oligonucleotide Synthesis (N. Gait, ed., Current Edition); Nucleic Acid Hybridization (B. Hames & S. Higgins, eds., Current Edition); Transcription and Translation (B. Hames & S. Higgins, eds., Current Edition); CRC Handbook of Parvoviruses, Vol. I & II (P. Tijessen, ed.); Fundamental Virology, 2nd Edition, Vol. I & II (B. N. Fields and D. M. Knipe, eds.)).

The markers of the invention may be detected by any method known to those of skill in the art, including without limitation LC-MS, GC-MS, immunoassays, hybridization and enzyme assays. The detection may be quantitative or qualitative. A wide variety of conventional techniques are available, including mass spectrometry, chromatographic separations, 2-D gel separations, binding assays (e.g., immunoassays), competitive inhibition assays, and so on. Any effective method in the art for measuring the presence/absence, level or activity of a polypeptide or polynucleotide is included in the invention. It is within the ability of one of ordinary skill in the art to determine which method would be most appropriate for measuring a specific marker. Thus, for example, a ELISA assay may be best suited for use in a physician's office while a measurement requiring more sophisticated instrumentation may be best suited for use in a clinical laboratory. Regardless of the method selected, it is important that the measurements be reproducible.

The markers of the invention can be measured by mass spectrometry, which allows direct measurements of analytes with high sensitivity and reproducibility. A number of mass spectrometric methods are available. Electrospray ionization (ESI), for example, allows quantification of differences in relative concentration of various species in one sample against another; absolute quantification is possible by normalization techniques (e.g., using an internal standard). Matrix-assisted laser desorption ionization (MALDI) or the related SELDI® technology (Ciphergen, Inc.) also could be used to make a determination of whether a marker was present, and the relative or absolute level of the marker. Mass spectrometers that allow time-of-flight (TOF) measurements have high accuracy and resolution and are able to measure low abundant species, even in complex matrices like serum or CSF.

For protein markers, quantification can be based on derivatization in combination with isotopic labeling, referred to as isotope coded affinity tags (“ICAT”). In this and other related methods, a specific amino acid in two samples is differentially and isotopically labeled and subsequently separated from peptide background by solid phase capture, wash and release. The intensities of the molecules from the two sources with different isotopic labels can then be accurately quantified with respect to one another. Quantification can also be based on the isotope dilution method by spiking in an isotopically labeled peptide or protein analogous to those being measured. Furthermore, quantification can also be determined without isotopic standards using the direct intensity of the analyte comparing with another measurement of a standard in a similar matrix.

In addition, one- and two-dimensional gels have been used to separate proteins and quantify gels spots by silver staining, fluorescence or radioactive labeling. These differently stained spots have been detected using mass spectrometry, and identified by tandem mass spectrometry techniques.

In one embodiment, the markers are measured using mass spectrometry in connection with a separation technology, such as liquid chromatography-mass spectrometry or gas chromatography-mass spectrometry. In particular, coupling reverse-phase liquid chromatography to high resolution, high mass accuracy ESI time-of-flight (TOF) mass spectroscopy allows spectral intensity measurement of a large number of biomolecules from a relatively small amount of any complex biological material. Analyzing a sample in this manner allows the marker (characterized by a specific RT and m/z) to be determined and quantified.

As will be appreciated by one of skill in the art, many other separation technologies may be used in connection with mass spectrometry. For example, a wide selection of separation columns is commercially available. In addition, separations may be performed using custom chromatographic surfaces (e.g., a bead on which a marker specific reagent has been immobilized). Molecules retained on the media subsequently may be eluted for analysis by mass spectrometry.

Analysis by liquid chromatography-mass spectrometry produces a mass intensity spectrum, the peaks of which represent various components of the sample, each component having a characteristic mass-to-charge ratio (m/z) and retention time (RT). The presence of a peak with the m/z and RT of a marker indicates that the marker is present. The peak representing a marker may be compared to a corresponding peak from another spectrum (e.g., from a control sample) to obtain a relative measurement. Any normalization technique in the art (e.g., an internal standard) may be used when a quantitative measurement is desired. “Deconvoluting” software is available to separate overlapping peaks. The retention time depends to some degree on the conditions employed in performing the liquid chromatography separation. The preferred conditions, those used to obtain the retention times that appear in the Tables, are set forth in the Example. The mass spectrometer preferably provides high mass accuracy and high mass resolution. The mass accuracy of a well-calibrated Micromass TOF instrument, for example, is reported to be approximately 5 mDa, with resolution m/Am exceeding 5000.

In other preferred embodiments, the level of the markers may be determined using a standard immunoassay, such as sandwiched ELISA using matched antibody pairs and chemiluminescent detection. Commercially available or custom monoclonal or polyclonal antibodies are typically used. However, the assay can be adapted for use with other reagents that specifically bind to the marker. Standard protocols and data analysis are used to determine the marker concentrations from the assay data.

A number of the assays discussed above employ a reagent that specifically binds to the marker. Any molecule that is capable of specifically binding to a marker is included within the invention. In some embodiments, the binding molecules are antibodies or antibody fragments. In other embodiments, the binding molecules are non-antibody species, such as aptamers. Thus, for example, the binding molecule may be an enzyme for which the marker is a substrate. The binding molecules may recognize any epitope of the targeted markers.

As described above, the binding molecules may be identified and produced by any method accepted in the art. Methods for identifying and producing antibodies and antibody fragments specific for an analyte are well known. Examples of other methods used to identify the binding molecules include binding assays with random peptide libraries (e.g., phage display) and design methods based on an analysis of the structure of the marker.

The markers of the invention also may be detected or measured using a number of chemical derivatization or reaction techniques known in the art. Reagents for use in such techniques are known in the art, and are commercially available for certain classes of target molecules.

Finally, the chromatographic separation techniques described above also may be coupled to an analytical technique other than mass spectrometry such as fluorescence detection of tagged molecules, NMR, capillary UV, evaporative light scattering or electrochemical detection.

Measurement of the relative amount of an RNA or protein marker of the invention may be by any method known in the art (see, e.g., Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989; and Current Protocols in Molecular Biology, eds. Ausubel et al. John Wiley & Sons: 1992). Typical methodologies for RNA detection include RNA extraction from a cell or tissue sample, followed by hybridization of a labeled probe (e.g., a complementary polynucleotide) specific for the target RNA to the extracted RNA, and detection of the probe (e.g., Northern blotting). Typical methodologies for protein detection include protein extraction from a cell or tissue sample, followed by hybridization of a labeled probe (e.g., an antibody) specific for the target protein to the protein sample, and detection of the probe. The label group can be a radioisotope, a fluorescent compound, an enzyme, or an enzyme co-factor. Detection of specific protein and polynucleotides may also be assessed by gel electrophoresis, column chromatography, direct sequencing, or quantitative PCR (in the case of polynucleotides) among many other techniques well known to those skilled in the art.

Detection of the presence or number of copies of all or a part of a marker gene of the invention may be performed using any method known in the art. Typically, it is convenient to assess the presence and/or quantity of a DNA or cDNA by Southern analysis, in which total DNA from a cell or tissue sample is extracted, is hybridized with a labeled probe (e.g., a complementary DNA molecule), and the probe is detected. The label group can be a radioisotope, a fluorescent compound, an enzyme, or an enzyme co-factor. Other useful methods of DNA detection and/or quantification include direct sequencing, gel electrophoresis, column chromatography, and quantitative PCR, as is known by one skilled in the art.

Polynucleotide similarity can be evaluated by hybridization between single stranded nucleic acids with complementary or partially complementary sequences. Such experiments are well known in the art. High stringency hybridization and washing conditions, as referred to herein, refer to conditions which permit isolation of nucleic acid molecules having at least about 80% nucleic acid sequence identity with the nucleic acid molecule being used to probe in the hybridization reaction (i.e., conditions permitting about 20% or less mismatch of nucleotides). Very high stringency hybridization and washing conditions, as referred to herein, refer to conditions which permit isolation of nucleic acid molecules having at least about 90% nucleic acid sequence identity with the nucleic acid molecule being used to probe in the hybridization reaction (i.e., conditions permitting about 10% or less mismatch of nucleotides). As discussed above, one of skill in the art can use the formulae in Meinkoth et al., ibid. to calculate the appropriate hybridization and wash conditions to achieve these particular levels of nucleotide mismatch. Such conditions will vary, depending on whether DNA:RNA or DNA:DNA hybrids are being formed. Calculated melting temperatures for DNA:DNA hybrids are 10° C. less than for DNA:RNA hybrids. In particular embodiments, stringent hybridization conditions for DNA:DNA hybrids include hybridization at an ionic strength of 6×SSC (0.9 M Na⁺) at a temperature of between about 20° C. and about 35° C. (lower stringency), more preferably, between about 28° C. and about 40° C. (more stringent), and even more preferably, between about 35° C. and about 45° C. (even more stringent), with appropriate wash conditions. In particular embodiments, stringent hybridization conditions for DNA:RNA hybrids include hybridization at an ionic strength of 6×SSC (0.9 M Na⁺) at a temperature of between about 30° C. and about 45° C., more preferably, between about 38° C. and about 50° C., and even more preferably, between about 45° C. and about 55° C., with similarly stringent wash conditions. These values are based on calculations of a melting temperature for molecules larger than about 100 nucleotides, 0% formamide and a G+C content of about 40%. Alternatively, Tₘ can be calculated empirically as set forth in Sambrook et al., supra, pages 9.31 to 9.62. In general, the wash conditions should be as stringent as possible, and should be appropriate for the chosen hybridization conditions. For example, hybridization conditions can include a combination of salt and temperature conditions that are approximately 20-25° C. below the calculated Tₘ of a particular hybrid, and wash conditions typically include a combination of salt and temperature conditions that are approximately 12-20° C. below the calculated Tₘ of the particular hybrid. One example of hybridization conditions suitable for use with DNA:DNA hybrids includes a 2-24 hour hybridization in 6×SSC (50% formamide) at about 42° C., followed by washing steps that include one or more washes at room temperature in about 2×SSC, followed by additional washes at higher temperatures and lower ionic strength (e.g., at least one wash as about 37° C. in about 0.1×-0.5×SSC, followed by at least one wash at about 68° C. in about 0.1×-0.5×SSC). Other hybridization conditions, and for example, those most useful with nucleic acid arrays, will be known to those of skill in the art.

The present invention also includes methods of diagnosing renal clear-cell cancer and related methods. In general, it is expected that the biomarkers described herein will be measured in combination with other signs, symptoms and clinical tests of renal clear-cell cancer, such as MRI or ultrasound abnormalities, or renal clear-cell cancer biomarkers reported in the literature. Likewise, more than one of the biomarkers of the present invention may be measured in combination. Measurement of the biomarkers of the invention along with any other markers known in the art, including those not specifically listed herein, falls within the scope of the present invention. Markers appropriate for this embodiment include those that have been identified as increased or decreased in samples obtained from renal clear-cell cancer samples compared with samples from non-renal clear-cell cancer samples (e.g., markers described in Tables 1-2), as well as antibodies produced by a patient in response to an increased level of a polypeptide marker. Other markers appropriate for this embodiment include fragments, precursors, successors and modified versions of such markers, polypeptides having substantial sequence identity to such markers, components having an m/z value and RT value of about the values set forth for the markers described in Tables 1-2, and molecules comprise one of the foregoing. Other appropriate markers for this embodiment will be apparent to one of skill in the art in light of the disclosure herein.

In one embodiment, the present invention provides a method for determining whether a subject has renal clear-cell cancer. In another aspect, the invention provides methods for diagnosing renal clear-cell cancer in a subject. These methods comprise obtaining a biological sample from a subject suspected of having renal clear-cell cancer, or at risk for developing renal clear-cell cancer, detecting the level or activity of one or more biomarkers in the sample, and comparing the result to the level or activity of the marker(s) in a sample obtained from a non-renal clear-cell cancer subject, or to a reference range or value. As used herein, the term “biological sample” includes a sample from any body fluid or tissue (e.g., serum, plasma, blood, cerebrospinal fluid, urine, kidney tissue). Typically, the standard biomarker level or reference range is obtained by measuring the same marker or markers in a set of normal controls. Measurement of the standard biomarker level or reference range need not be made contemporaneously; it may be a historical measurement. Preferably the normal control is matched to the patient with respect to some attribute(s) (e.g., age). Depending upon the difference between the measured and standard level or reference range, the patient can be diagnosed as having renal clear-cell cancer or as not having renal clear-cell cancer. In some embodiments, renal clear-cell cancer is diagnosed in the patient if the expression level of the biomarker or biomarkers in the patient sample is statistically more similar to the expression level of the biomarker or biomarkers that has been associated with renal clear-cell cancer than the expression level of the biomarker or biomarkers that has been associated with the normal controls.

What is presently referred to as renal clear-cell cancer may turn out to be a number of related, but distinguishable conditions. Classifications may be made, and these types may be further distinguished into subtypes. Any and all of the various forms of renal clear-cell cancer are intended to be within the scope of the present invention. Indeed, by providing a method for subsetting patients based on biomarker measurement level, the compositions and methods of the present invention may be used to uncover and define various forms of the disease.

The methods of the present invention may be used to make the diagnosis of renal clear-cell cancer, independently from other information such as the patient's symptoms or the results of other clinical or paraclinical tests. However, the methods of the present invention may be used in conjunction with such other data points.

Because a diagnosis is rarely based exclusively on the results of a single test, the method may be used to determine whether a subject is more likely than not to have renal clear-cell cancer, or is more likely to have renal clear-cell cancer than to have another disease, based on the difference between the measured and standard level or reference range of the biomarker. Thus, for example, a patient with a putative diagnosis of renal clear-cell cancer may be diagnosed as being “more likely” or “less likely” to have renal clear-cell cancer in light of the information provided by a method of the present invention. If a plurality of biomarkers are measured, at least one and up to all of the measured biomarkers must differ, in the appropriate direction, for the subject to be diagnosed as having (or being more likely to have) renal clear-cell cancer. In some embodiments, such difference is statistically significant.

The biological sample may be of any tissue or fluid, including a serum or tissue sample, but other biological fluids or tissue may be used. Possible biological fluids include, but are not limited to, plasma, urine and kidney tissue. In some embodiments, the level of a marker may be compared to the level of another marker or some other component in a different tissue, fluid or biological “compartment.” Thus, a differential comparison may be made of a marker in tissue and serum. It is also within the scope of the invention to compare the level of a marker with the level of another marker or some other component within the same compartment.

As will be apparent to those of ordinary skill in the art, the above description is not limited to making an initial diagnosis of renal clear-cell cancer, but also is applicable to confirming a provisional diagnosis of renal clear-cell cancer or “ruling out” such a diagnosis. Furthermore, an increased or decreased level or activity of the marker(s) in a sample obtained from a subject suspected of having renal clear-cell cancer, or at risk for developing renal clear-cell cancer, is indicative that the subject has or is at risk for developing renal clear-cell cancer.

The invention also provides a method for determining a subject's risk of developing renal clear-cell cancer, the method comprising obtaining a biological sample from a subject, detecting the level or activity of a marker in the sample, and comparing the result to the level or activity of the marker in a sample obtained from a non- renal clear-cell cancer subject, or to a reference range or value wherein an increase or decrease of the marker is correlated with the risk of developing renal clear-cell cancer.

The invention also provides methods for determining the stage or severity of renal clear-cell cancer, the method comprising obtaining a biological sample from a subject, detecting the level or activity of a marker in the sample, and comparing the result to the level or activity of the marker in a sample obtained from a non-renal clear-cell cancer subject, or to a reference range or value wherein an increase or decrease of the marker is correlated with the stage or severity of the disease.

In another aspect, the invention provides methods for monitoring the progression of the disease in a subject who has renal clear-cell cancer, the method comprising obtaining a first biological sample from a subject, detecting the level or activity of a marker in the sample, and comparing the result to the level or activity of the marker in a second sample obtained from the subject at a later time, or to a reference range or value wherein an increase or decrease of the marker is correlated with progression of the disease.

As indicated in Tables 1-2, some of the marker measurement values are higher in renal clear-cell cancer samples, while others are lower. A significant difference in the appropriate direction in the measured value of one or more of the markers indicates that the patient has (or is more likely to have, or is at risk of having, or is at risk of developing, and so forth) renal clear-cell cancer. If only one biomarker is measured, then that value must increase or decrease to indicate renal clear-cell cancer. If more than one biomarker is measured, then a diagnosis of renal clear-cell cancer can be indicated by a change in only one biomarker, all biomarkers, or any number in between. In some embodiments, multiple markers are measured, and a diagnosis of renal clear-cell cancer is indicated by changes in multiple markers. For example, a panel of markers may include markers that are increased in level or activity in renal clear-cell cancer subject samples as compared to non-renal clear-cell cancer subject samples, markers that are decreased in level or activity in renal clear-cell cancer subject samples as compared to non- renal clear-cell cancer subject samples, or a combination thereof. Measurements can be of (i) a biomarker of the present invention, (ii) a biomarker of the present invention and another factor known to be associated with renal clear-cell cancer (e.g., MRI scan); (iii) a plurality of biomarkers of the present invention, (iv) a plurality of biomarkers comprising at least one biomarker of the present invention and at least one biomarker reported in the literature (e.g., VEGF) or (v) any combination of the foregoing. Furthermore, the amount of change in a biomarker level may be an indication of the relatively likelihood of the presence of the disease.

The marker may be detected in any biological sample obtained from the subject, by any suitable method known in the art (e.g., immunoassays, hybridization assay) see supra.

In an alternative embodiment of the invention, a method is provided for monitoring a renal clear-cell cancer patient over time to determine whether the disease is progressing. The specific techniques used in implementing this embodiment are similar to those used in the embodiments described above. The method is performed by obtaining a biological sample, such as serum or tissue, from the subject at a certain time (t₁); measuring the level of at least one of the biomarkers in the biological sample; and comparing the measured level with the level measured with respect to a biological sample obtained from the subject at an earlier time (t₀). Depending upon the difference between the measured levels, it can be seen whether the marker level has increased, decreased, or remained constant over the interval (t₁-t₀). A further deviation of a marker in the direction indicating renal clear-cell cancer, or the measurement of additional increased or decreased renal clear-cell cancer markers, would suggest a progression of the disease during the interval. Subsequent sample acquisitions and measurements can be performed as many times as desired over a range of times t₂ to tₙ.

The ability to monitor a patient by making serial marker level determinations would represent a valuable clinical tool. Rather than the limited “snapshot” provided by a single test, such monitoring would reveal trends in marker levels over time. In addition to indicating a progression of the disease, tracking the marker levels in a patient could be used to predict exacerbations or indicate the clinical course of the disease. For example, as will be apparent to one of skill in the art, the biomarkers of the present invention could be further investigated to distinguish between any or all of the known forms of renal clear-cell cancer or any later described types or subtypes of the disease. In addition, the sensitivity and specificity of any method of the present invention could be further investigated with respect to distinguishing renal clear-cell cancer from other diseases or to predict relapse or remission.

In an analogous manner, administration routes of a particular drug can be examined. The drug can be administered differently to different subject populations, and measurements corresponding to each administration route analyzed to determined if the differences in the inventive biomarkers before and after drug administration are significant. Results from the different routes can also be compared with each other directly.

In another aspect, the invention provides methods for screening candidate compounds for use as therapeutic compounds. In one embodiment, the method comprises screening candidate compounds for those that bind to a polypeptide or polynucleotide molecule of the invention. Candidate compounds that bind to markers can be identified using any suitable method or technique known in the art.

In one embodiment, a candidate compound or a control is contacted with marker and the ability of the candidate compound to form stable complexes is determined (e.g., flow cytometry, immunoprecipitation). The candidate compound, the marker, or an antibody that specifically binds either may be labeled to facilitate detection. The candidate molecule or marker may be immobilized on a solid support (e.g., a bead).

In another embodiment, cells expressing a polypeptide marker are contacted with a candidate compound or a control and the ability of the candidate compound to form stable complexes with the cells is determined. The candidate compound or the marker may be labeled to facilitate detection.

In an analogous manner, the markers of the present invention can be used to assess the efficacy of a therapeutic intervention in a subject. The same approach described above would be used, except a suitable treatment would be started, or an ongoing treatment would be changed, before the second measurement (i.e., after t₀ and before t₁). The treatment can be any therapeutic intervention, such as drug administration, dietary restriction or surgery, and can follow any suitable schedule over any time period as appropriate for the intervention. The measurements before and after could then be compared to determine whether or not the treatment had an effect effective. As will be appreciated by one of skill in the art, the determination may be confounded by other superimposed processes (e.g., an exacerbation of the disease during the same period).

In a further additional embodiment, the markers may be used to screen candidate drugs, for example, in a clinical trial, to determine whether a candidate drug is effective in treating renal clear-cell cancer. At time t₀, a biological sample is obtained from each subject in population of subjects diagnosed with renal clear-cell cancer. Next, assays are performed on each subject's sample to measure levels of a biological marker. In some embodiments, only a single marker is monitored, while in other embodiments, a combination of markers, up to the total number of factors, is monitored. Next, a predetermined dose of a candidate drug is administered to a portion or sub-population of the same subject population. Drug administration can follow any suitable schedule over any time period. In some cases, varying doses are administered to different subjects within the sub-population, or the drug is administered by different routes. At time t₁, after drug administration, a biological sample is acquired from the sub-population and the same assays are performed on the biological samples as were previously performed to obtain measurement values. As before, subsequent sample acquisitions and measurements can be performed as many times as desired over a range of times t₂ to t₀. In such a study, a different sub-population of the subject population serves as a control group, to which a placebo is administered. The same procedure is then followed for the control group: obtaining the biological sample, processing the sample, and measuring the biological markers to obtain a measurement chart.

Specific doses and delivery routes can also be examined. The method is performed by administering the candidate drug at specified dose or delivery routes to subjects with renal clear-cell cancer; obtaining biological samples, such as serum or tissue, from the subjects; measuring the level of at least one of the biomarkers in each of the biological samples; and, comparing the measured level for each sample with other samples and/or a standard level. Typically, the standard level is obtained by measuring the same marker or markers in the subject before drug administration. Depending upon the difference between the measured and standard levels, the drug can be considered to have an effect on renal clear-cell cancer. If multiple biomarkers are measured, at least one and up to all of the biomarkers must change, in the expected direction, for the drug to be considered effective. Preferably, multiple markers must change for the drug to be considered effective, and preferably, such change is statistically significant.

As will be apparent to those of ordinary skill in the art, the above description is not limited to a candidate drug, but is applicable to determining whether any therapeutic intervention is effective in treating renal clear-cell cancer.

In a typical embodiment, a subject population having renal clear-cell cancer is selected for the study. The population is typically selected using standard protocols for selecting clinical trial subjects. For example, the subjects are generally healthy, are not taking other medication, and are evenly distributed in age and sex. The subject population can also be divided into multiple groups; for example, different sub-populations may be suffering from different types or different degrees of the disorder to which the candidate drug is addressed. The stratification of the patient population may be made based on the levels of biomarkers of the present invention.

In general, a number of statistical considerations must be made in designing the trial to ensure that statistically significant changes in biomarker measurements can be detected following drug administration. The amount of change in a biomarker depends upon a number of factors, including strength of the drug, dose of the drug, and treatment schedule. It will be apparent to one skilled in statistics how to determine appropriate subject population sizes. Preferably, the study is designed to detect relatively small effect sizes.

The subjects optionally may be “washed out” from any previous drug use for a suitable period of time. Washout removes effects of any previous medications so that an accurate baseline measurement can be taken. At time t₀, a biological sample is obtained from each subject in the population. Next, an assay or variety of assays is performed on each subject's sample to measure levels of particular biomarkers of the invention. The assays can use conventional methods and reagents, as described above. If the sample is blood, then the assays typically are performed on either serum or plasma. For other fluids or tissues, additional sample preparation steps are included as necessary before the assays are performed. The assays measure values of at least one of the biological markers described herein. In some embodiments, only a single marker is monitored, while in other embodiments, a combination of factors, up to the total number of markers, is monitored. The markers may also be monitored in conjunction with other measurements and factors associated with renal clear-cell cancer (e.g., MRI imaging). The number of biological markers whose values are measured depends upon, for example, the availability of assay reagents, biological fluid, and other resources.

Next, a predetermined dose of a candidate drug is administered to a portion or sub-population of the same subject population. Drug administration can follow any suitable schedule over any time period, and the sub-population can include some or all of the subjects in the population. In some cases, varying doses are administered to different subjects within the sub-population, or the drug is administered by different routes. Suitable doses and administration routes depend upon specific characteristics of the drug. At time t₁, after drug administration, another biological sample (the “t₁ sample”) is acquired from the sub-population. Typically, the sample is the same type of sample and processed in the same manner as the sample acquired from the subject population before drug administration (the “t₀ sample”). The same assays are performed on the t₁ sample as on the t₀ sample to obtain measurement values. Subsequent sample acquisitions and measurements can be performed as many times as desired over a range of times t₂ to tₙ.

Typically, a different sub-population of the subject population is used as a control group, to which a placebo is administered. The same procedure is then followed for the control group: obtaining the biological sample, processing the sample, and measuring the biological markers to obtain measurement values. Additionally, different drugs can be administered to any number of different sub-populations to compare the effects of the multiple drugs. As will be apparent to those of ordinary skill in the art, the above description is a highly simplified description of a method involving a clinical trial. Clinical trials have many more procedural requirements, and it is to be understood that the method is typically implemented following all such requirements.

Paired measurements of the various biomarkers are now available for each subject. The different measurement values are compared and analyzed to determine whether the biological markers changed in the expected direction for the drug group but not for the placebo group, indicating that the candidate drug is effective in treating the disease. In preferred embodiments, such change is statistically significant. The measurement values at time t₁ for the group that received the candidate drug are compared with standard measurement values, preferably the measured values before the drug was given to the group, i.e., at time t₀. Typically, the comparison takes the form of statistical analysis of the measured values of the entire population before and after administration of the drug or placebo. Any conventional statistical method can be used to determine whether the changes in biological marker values are statistically significant. For example, paired comparisons can be made for each biomarker using either a parametric paired t-test or a non-parametric sign or sign rank test, depending upon the distribution of the data.

In addition, tests may be performed to ensure that statistically significant changes found in the drug group are not also found in the placebo group. Without such tests, it cannot be determined whether the observed changes occur in all patients and are therefore not a result of candidate drug administration.

As indicated in Tables 1-2, some of the marker measurement values are higher in samples from renal clear-cell cancer patients, while others are lower. The nonadjusted p-values shown were obtained by univariate analysis. A significant change in the appropriate direction in the measured value of one or more of the markers indicates that the drug is effective. If only one biomarker is measured, then that value must increase or decrease to indicate drug efficacy. If more than one biomarker is measured, then drug efficacy can be indicated by change in only one biomarker, all biomarkers, or any number in between. In some embodiments, multiple markers are measured, and drug efficacy is indicated by changes in multiple markers. Measurements can be of both biomarkers of the present invention and other measurements and factors associated with renal clear-cell cancer (e.g., measurement of biomarkers reported in the literature and/or MRI imaging). Furthermore, the amount of change in a biomarker level may be an indication of the relatively efficacy of the drug.

In addition to determining whether a particular drug is effective in treating renal clear-cell cancer, biomarkers of the invention can also be used to examine dose effects of a candidate drug. There are a number of different ways that varying doses can be examined. For example, different doses of a drug can be administered to different subject populations, and measurements corresponding to each dose analyzed to determine if the differences in the inventive biomarkers before and after drug administration are significant. In this way, a minimal dose required to effect a change can be estimated. In addition, results from different doses can be compared with each other to determine how each biomarker behaves as a function of dose. Based on the results of drug screenings, the markers of the invention may be used as theragnostics; that is, they can be used to individualize medical treatment.

In another aspect, the invention provides a kit for detecting a polypeptide or polynucleotide marker.

In another aspect, the invention provides a kit for diagnosing renal clear-cell cancer in a patient including reagents for detecting at least one polypeptide or polynucleotide marker in a biological sample from the subject.

In another aspect, the invention provides a kit for screening candidate compounds including reagents for detecting stable complexes between the candidate compound and a polynucleotide or polynucleotide marker.

The kits of the invention may comprise one or more of the following: an antibody, wherein the antibody specifically binds with a polypeptide marker, a labeled binding partner to the antibody, a solid phase upon which is immobilized the antibody or its binding partner, a polynucleotide probe that can hybridize to a polynucleotide marker, pairs of primers that under appropriate reaction conditions can prime amplification of at least a portion of a polynucleotide marker or a polynucleotide encoding a polypeptide marker (e.g., by PCR), instructions on how to use the kit, and a label or insert indicating regulatory approval for diagnostic or therapeutic use.

The invention further includes polynucleotide or polypeptide microarrays comprising polypeptides of the invention, polynucleotides of the invention, or molecules, such as antibodies, which specifically bind to the polypeptides or polynucleotides of the present invention. In this aspect of the invention, standard techniques of microarray technology are utilized to assess expression of the polypeptides biomarkers and/or identify biological constituents that bind such polypeptides. Protein microarray technology is well known to those of ordinary skill in the art and is based on, but not limited to, obtaining an array of identified peptides or proteins on a fixed substrate, binding target molecules or biological constituents to the peptides, and evaluating such binding. Polynucleotide arrays, particularly arrays that bind polypeptides of the invention, also can be used for diagnostic applications, such as for identifying subjects that have a condition characterized by expression of polypeptide biomarkers, e.g., renal clear-cell cancer.

The invention also provides methods for treating renal clear-cell cancer, as well as other diseases or conditions, by providing a therapeutic agent to a subject that increases or decreases the level or activity of at least one marker of the invention.

In one embodiment, the method comprises administering a therapeutic agent to a subject that increases level or activity of at least one polypeptide or polynucleotide marker of the invention that is decreased in samples obtained from renal clear-cell cancer subjects compared to samples obtained from non- renal clear-cell cancer subjects or to a reference range or value.

In another embodiment, the method comprises administering a therapeutic agent to a subject that decreases the level of at least one polypeptide or polynucleotide marker of the invention that is increased in samples obtained from renal clear-cell cancer subjects compared to samples obtained from non- renal clear-cell cancer subjects or to a reference range or value.

In another embodiment, the method further comprises first obtaining a sample from an renal clear-cell cancer subject, determining the presence, level or activity of at least one marker of the invention in the sample compared to samples obtained from a non-renal clear-cell cancer subject or to a reference range or value. If the marker is increased in the sample obtained from the renal clear-cell cancer subject, a therapeutic agent that decreases the level of the marker is administered to the patient. If the marker is decreased in the sample obtained from the renal clear-cell cancer subject, a therapeutic agent that increases the level of the marker is administered to the subject.

Therapeutic agents include but are not limited to polypeptide markers, polynucleotide markers, molecules comprising a polypeptide marker or polynucleotide marker, antibodies to polypeptide marker or polynucleotide marker, modulators of the level or activity a polypeptide or polynucleotide marker (e.g., an inhibitor, anti-sense polynucleotides) or compositions comprising one or more of the foregoing.

Generally, the therapeutic agents used in the invention are administered to the subject in an effective amount. An “effective amount” is typically the amount that is sufficient to obtain beneficial or desired clinical results. The effective amount is generally determined by a physician with respect to a specific patient and is within the skill of one in the art. Factors that may be taken into account in determining an effective amount include those relating to the condition being treated (e.g., type, stage, severity) as well as those relating to the subject (e.g., age, weight).

The level or activity of a polypeptide marker may be increased or decreased by any suitable technique or method known in the art. The level of a polypeptide marker may be increased by providing the polypeptide marker to a subject. Alternatively, the level of a polypeptide marker may be increased by providing a polynucleotide that encodes the polypeptide marker (e.g., gene therapy). For those polypeptide markers with enzymatic activity, compounds or molecules known to increase that activity may be provided to the subject.

The level of a polypeptide marker may be decreased by providing antibodies specific for the polypeptide marker to the subject. Alternatively, the level of a polypeptide marker may be decreased by providing a polynucleotide that is “anti-sense” to the polynucleotide that encodes the polypeptide marker, or that encodes dysfunctional proteins. For those polypeptide markers with enzymatic activity, compounds or molecules known to decrease that activity (e.g., inhibitor or antagonist).

The therapeutic compounds described herein may be administered alone or in combination with another therapeutic compound, or other form of treatment. The compounds may be administered to the subjects in any suitable manner known in the art (e.g., orally, topically, subcutaneously, intradermally, intramuscularly, intravenously, intraarterially, intrathecally). Metabolites may be combined with an excipient and formulated as tablets or capsules for oral administration. Polypeptides may be formulated for parenteral administeration to avoid denaturation by stomach acids. For polynucleotides, vectors may be constructed for administration to the subject by a virus or other carrier. In a typical embodiment, cDNA is delivered to target cells (e.g., bone marrow cells) that are later reintroduced into the subject for expression of the encoded protein. A therapeutic composition can be administered in a variety of unit dosage forms depending upon the method of administration.

### EXAMPLES

### Example 1

Sample Selection. Samples were collected from 15 human subjects diagnosed with renal clear-cell cancer. The samples consisted of plasma from the patients obtained before and after surgery to remove the affected kidney, to remove the carcinoma. Comparisons were made before (disease) surgery versus three months after (presumed disease-free) surgery.

Plasma Proteome. A high molecular weight fraction (“plasma proteome”) was analyzed from the plasma samples. The plasma (50 microliters) was diluted 1:5 with “Buffer A” from Agilent Technologies, Inc. (Palo Alto, Calif.) as part of their multiple affinity removal system (MARS) antibody-based depletion system for the purpose of increasing the effective dynamic range of the measurements. For this human plasma, the six most abundant proteins (plasma albumin, IgG, IgA, transferrin, antitrypsin and haptoglobin) were substantially depleted by an affinity (solid-phase-bound-antibody) column (Agilent Technologies, Inc., Palo Alto, Calif.). The remaining proteins were denatured using 6M guanidinium hydrochloride in trishydroxymethylaminomethane buffer (pH 8.3), disulfide bonds were reduced using 10 mM dithiothreitol (4 hr, 37 C), and then sulfhydryl groups carboxymethylated with 25 mM iodoacetic acid solution neutralized with NaOH (30 min at room temperature). The denaturant and reduction-alkylation reagents were removed by buffer exchange with 50 mM ammonium bicarbonate buffer (pH 8.3) using a 9-kDa molecular weight cut-off spin filter (Orbital Biosciences, Topsfield, Mass.). After digestion of the proteins with modified trypsin (Promega Corp., Madison, Wis.) incubated for 22 hours at 37 C, the mixture was acidified with 1% formic acid, desalted with a C18 solid-phase extraction (SPE) cartridge (Sep/Pak cartridge by Waters Corp., Milford, Mass.), dried, and re-dissolved in 110 microliters of SCX-buffer-A which consists of 20 mM KH₂PO₄ with 25% acetonitrile acidified with formic acid to pH 3.

Strong-cation exchange (SCX) chromatography. The samples, each dissolved in SCX-buffer-A, are injected onto a 2-mm diameter×250-mm long Spherisorb SCX column, with 5-micron diameter particles. A gradient elutes the analytes over time, with A buffer being the SCX-buffer-A, and B buffer consisting of SCX-buffer-A plus 500 mM KCl. The eluent is collected on a fraction collector and for this example, four fractions were collected. Each of these fractions were then dried, re-dissolved in 5 mL of 0.1% formic acid, desalted with a C18 solid-phase extraction (SPE) cartridge (Sep/Pak cartridge by Waters Corp., Milford, Mass.), dried, and re-dissolved in 50 microliters of 0.1% formic acid. Ten microliters of this solution was injected for LC-MS Analysis.

LC-MS Analysis. Tryptic and non-tryptic peptides were profiled by liquid chromatography-electrospray ionization-mass spectrometry (LC-ESI-MS) on a high-resolution time-of-flight (TOF) instrument. For LC separation, an online 0.3 mm diameter×15 cm long column was packed with C18 reverse-phase (RP) material (Micro-Tech Scientific, Inc., Vista, Calif.). Peptides retained on the RP column were eluted with increasing concentration of acetonitrile (ACN). The eluate from the column flowed into the ESI-TOF MS (Micromass LCT™, Waters Corp., Milford, Mass.). Individual molecules were tracked across samples and their differential expression determined.

### Example 2

Plasma Peptidome. The lower molecular-weight polypeptide fraction was analyzed using 0.5 mL of human plasma diluted 1:5 by volume with SEC-buffer-A which consisted of 20 mM KH₂PO₄ with 20% acetonitrile acidified with formic acid to pH 3. This solution is split into 5 parts, and each part independently passed through a molecular-weight cut-off filter, Amicon 50 kDa cut-off spin filter (Millipore Corp., Bedford, Mass.) using an acceleration of 1500×g for approximately 100 minutes. The flow-through solution is then pooled for each sample in a glass tube and then dried, and re-dissolved in 200 microliters of SEC-buffer-A.

Size-exclusion (SEC) chromatography. The solution for SEC chromatography was injected onto two Sperdex™ SEC peptide columns in tandem, each 10-mm diameter×300-mm long, and fraction collected into four fractions spanning the molecular weight range up to roughly 20,000 kDa.

The first fraction, containing the highest molecular weight polypeptides, was digested by addition of 200 microliters of 5 mM of tris(2-carboxyethyl)phosphine (TCEP), 0.1% of RapiGest™ SF (which assists to denature proteins for digestion, Waters Corp., Milford, Mass.) in 50 mM NH₄HCO₃ at pH 8.3 and incubated at 37 C for one hour. This solution is then incubated at 5:1 by weight with modified trypsin (Promega, Madison, Wis.) at 37 C for 16 hours. This solution is then acidified with 100 microliters of 0.5 M HCl and incubated at 37 C for 45 minutes to break-up the RapiGest™ SF. This solution is then centrifuged at 13,000 RPM and the subsequent supernatant is removed by pipetting and added to 300 microliters of H₂O. This solution is then desalted with a C18 solid-phase extraction (SPE) cartridge (Sep/Pak cartridge by Waters Corp., Milford, Mass.), dried, and re-dissolved in 50 microliters of aqueous solution of 1 mM TCEP and 0.1% formic acid incubated for 37 for one hour. Twenty microliters of this solution was then injected for LC-MS Analysis.

Fractions 2 through 4 were not digested, and each was dried, re-dissolved in 1 mL of 0.1% formic acid, desalted with a C18 solid-phase extraction (SPE) cartridge (Sep/Pak cartridge by Waters Corp., Milford, Mass.), dried, and re-dissolved in 50 microliters of 0.1% formic acid with 20 microliters injected for LC-MS Analysis.

LC-MS Analysis. Tryptic and non-tryptic peptides were profiled by liquid chromatography-electrospray ionization-mass spectrometry (LC-ESI-MS) on a high-resolution time-of-flight (TOF) instrument. For LC separation, an online 0.3 mm diameter×15 cm long column was packed with C18 reverse-phase (RP) material (Micro-Tech Scientific, Inc., Vista, Calif.). Peptides retained on the RP column were eluted with increasing concentration of acetonitrile (ACN). The eluate from the column flowed into the ESI-TOF MS (Micromass LCT™, Waters Corp., Milford, Mass.). Individual molecules were tracked across samples and their differential expression determined.

Peptide Identification Data Acquisition. MS/MS spectra obtained from the LTQ (Thermo Electron Corp., San Jose, Calif.) and Q-TOF (Waters Corp.) mass spectrometers were used to identify peptides. A more accurate parent ion molecular weight is obtained from a parallel analysis using the LCT orthogonal-injection ESI-TOF (Micromass). Accuracy of the LCT detection is as good as ˜10 ppm using the natural internal calibration of known peptides. In comparison, accuracy of the LTQ ion trap is ±0.5 Da (˜1000 ppm). This data is then examined by a database searching approach (described below). In addition, de novo amino acid sequence analysis programs can be used to obtain at least partial sequence analysis. Increased resolution (˜5,000) and accuracy of the LCT TOF instrument significantly limits the range of possible peptides that are candidates, thus allowing focused database searches; this is a valuable contribution for making correct identifications especially in the case of low signal-to-noise mass peaks.

Peptide and Protein Identification Data Analysis. TurboSEQUEST software (Thermo Electron Corp, San Jose, Calif.), or similar software such as Mascot (Matrix Science LTD, London, UK), is used to identify peptides and proteins. Link A J, et al. (1999) “Direct analysis of protein complexes using mass spectrometry.” Nature Biotechnol 17:676-82. TurboSEQUEST uses protein or DNA databases, both public and private. In the case of enzymatically digested proteins, an in silico digestion of the associated proteins produces peptides with amino acid sequences theoretically revealed by a computational cleavage according to known rules; these are used to compare against the raw data. Looking up a particular molecular weight with a given mass uncertainty gives a selection of possible peptides (and hence proteins) that can give rise to those peaks. The in silico digestion can include a small number of PTMs. However, database approaches such as TurboSEQUEST will not work to identify peptides or proteins that are not already in the database. In that case, de novo peptide sequencing software and BLAST searching can be used.

Post-Translational Modification (PTM). A number of methods were used to detect PTM of the identified polypeptides. Using the known fixed mass that a PTM adds, TurboSEQUEST or Mascot software can identify at least three PTMs on a peptide in a single search.

Differential Quantification. Proteins and peptides were quantified relative to the same, corresponding molecules in a different sample, usually a control or normal sample. This differential expression approach relies on the assumption that biological samples consist of complex mixtures of multiple biological components, of which only some are relevant to the comparison. The majority of components are relatively constant for the same individual over time or across subject populations. The majority of components whose concentrations do not vary across samples are used as an intrinsic internal standard to normalize the concentrations of components that do vary. The method also relies on the inherent reproducibility of ionization for ESI. The high reproducibility of ESI is measured by the coefficient of variation. The majority of peaks have a CV less then 20%, aside from biological variance. The validity of this approach is discussed in more detail in Wang et al. (2003) and U.S. Pat. No. 6,835,927.

Determination of p-Value. Univariate hypothesis tests for each mass spectrometry component were used for the comparisons of means between cancer-free and cancer groups. Parametric or non-parametric tests were used, depending on the normality of the data. If the data were approximately normally distributed, the parametric statistic was used (t-test); if not, the nonparametric statistic (Wilcoxon test) was used. Goodness-of-fit statistics (Shapiro-Wilk) and tests of skewness and kurtosis were performed to assess the normality of each biometric component. The results of these tests are presented in form of a p-value per component. The p-value represents the probability of a false positive on a univariate level.

Those skilled in the art will appreciate, or be able to ascertain using no more than routine experimentation, further features and advantages of the invention based on the above-described embodiments. Accordingly, the invention is not to be limited by what has been particularly shown and described, except as indicated by the appended claims. All publications and references are herein expressly incorporated by reference in their entirety.

LENGTHY TABLE REFERENCED HERE
US20070292869A1-20071220-T00001
Please refer to the end of the specification for access instructions.

LENGTHY TABLE REFERENCED HERE
US20070292869A1-20071220-T00002
Please refer to the end of the specification for access instructions.

LENGTHY TABLE REFERENCED HERE
US20070292869A1-20071220-T00003
Please refer to the end of the specification for access instructions.

LENGTHY TABLE
The patent application contains a lengthy table section. A copy of the table is available in electronic form from the USPTO web site (http://seqdata.uspto.gov/?pageRequest=docDetail&DocID=US20070292869A1). An electronic copy of the table will also be available from the USPTO upon request and payment of the fee set forth in 37 CFR 1.19(b)(3).

1. A method for diagnosing renal cell cancer in a subject, comprising:
determining the level of a marker in a biological sample obtained from a subject;
comparing the level of the marker in the sample to a reference value, wherein the marker is selected from the group consisting of a polypeptide comprising a marker identified in Tables 1-2, and a polynucleotide encoding a polypeptide comprising a marker identified in Tables 1-2.

2. The method of claim 1, wherein the marker is selected from the group consisting of a polypeptide identified in Tables 1-2.

3. The method of claim 2, wherein the marker is selected from the group consisting of a polypeptide identified in Table 1.

4. The method of claim 2, wherein the marker is selected from the group consisting of a polypeptide identified in Table 2.

5. The method of claim 1, wherein the biological sample is a body fluid.

6. The method of claim 5, wherein the body fluid is selected from the group consisting of blood, serum, plasma, cerebrospinal fluid, urine, and saliva.

7. The method of claim 1, wherein the biological sample is serum.

8. The method of claim 1, wherein the marker comprises a polypeptide or fragment thereof.

9. The method of claim 1, wherein the reference value is the level of the marker in at least one sample from a non-renal cell cancer subject.

10. The method of claim 1, wherein the polypeptide is the marker.

11. The method of claim 1, wherein the polypeptide shares at least about 70% sequence identity with the marker.

12. The method of claim 1, wherein the polypeptide is a modified form of the marker.

13. The method of claim 1, wherein the method further comprises detecting the presence of the polypeptide using a reagent that specifically binds to the polypeptide or a fragment thereof.

14. The method of claim 13, wherein the reagent is selected from the group consisting of an antibody, an antibody derivative, and an antibody fragment.

15. The method of claim 1, wherein the renal cell cancer is renal clear-cell cancer.

16. A method for diagnosing renal cell cancer in a subject, the method comprising:
determining the level of a plurality of markers in one or more biological samples from a subject, wherein at least two of the plurality of markers are selected from the group consisting of a polypeptide comprising a marker identified in Tables 1-2, and a polynucleotide encoding a polypeptide comprising a marker identified in Tables 1-2; and
comparing the level of at least two of the plurality of markers to a reference value.

17-36. (canceled)

37. A method for monitoring renal cell cancer in a subject, the method comprising:
measuring the level of a marker in first biological sample from a subject, wherein the marker is selected from the group consisting of a polypeptide comprising a marker identified in Tables 1-2, and a polynucleotide encoding a polypeptide comprising a marker identified in Tables 1-2;
measuring the level of the marker in a second biological sample from a subject, wherein the marker is selected from the group consisting of a polypeptide comprising a marker identified in Tables 1-2, and a polynucleotide encoding a polypeptide comprising a marker identified in Tables 1-2; and
comparing the level of the marker measured in the first sample with the level of the marker measured in the second sample, whereby renal cell cancer in a subject is monitored.

38-47. (canceled)

48. A method of assessing the efficacy of a treatment for renal cell cancer in a subject, the method comprising comparing:
the level of a marker measured in a first sample obtained from the subject before the treatment has been administered to the subject, wherein the marker is selected from the group consisting of a polypeptide comprising a marker identified in Tables 1-2, and a polynucleotide encoding a polypeptide comprising a marker identified in Tables 1-2, and the level of the marker in a second sample obtained from the subject after the treatment has been administered to the subject, wherein a change in the level of the marker in the second sample relative to the first sample is an indication that the treatment is efficacious for treating renal cell cancer in the subject.

49-58. (canceled)

57. A method for determining the risk of developing renal cell cancer in a subject, the method comprising:
obtaining a biological sample from the subject;
determining the level of a marker in the sample, wherein the marker is selected from the group consisting of a polypeptide comprising a marker identified in Tables 1-2, and a polynucleotide encoding a polypeptide comprising a marker identified in Tables 1-2;
comparing the level of the marker in the sample to a reference value; and
determining from the results of the comparison that the subject has an increased or decreased risk of developing renal cell cancer.

58-61. (canceled)

62. A method for diagnosing renal cell cancer in a subject, the method comprising:
obtaining a biological sample from the subject;
determining the level of a protein in the sample that specifically binds to a marker, wherein the marker is selected from the group consisting of a polypeptide comprising a marker identified in Tables 1-2, and a polynucleotide encoding a polypeptide comprising a marker identified in Tables 1-2, wherein renal cell cancer is diagnosed in the subject if the level of the biomarker in the patient sample is more similar to the level of the biomarker that has been associated with renal cell cancer than the level of the biomarker that has been associated with controls.

63-66. (canceled)
